SEQUENCE LISTING (16 sequences)

SEQ ID NO: 1 - protein, 9 aa, Homo sapiens
VFETLEEIT

SEQ ID NO: 2 - protein, 16 aa, Homo sapiens
YSQAVPAVTEGPIPEV

SEQ ID NO: 3 - protein, 33 aa, Artificial
  Biotin linked to N-terminal
VATYAGQFNQDYLSGMAANMSGTFGGANMPNLY

SEQ ID NO: 4 - protein, 25 aa, Artificial
  Biotin linked to N-terminal
EPPLSQETFSDLWKLLPENNVLSPL

SEQ ID NO: 5 - protein, 15 aa, Artificial
  Biotin linked to N-terminal
NHEPSVTQVILDRPY

SEQ ID NO: 6 - protein, 15 aa, Artificial
  Biotin linked to N-terminal
TVETPQTAKTAYYAT

SEQ ID NO: 7 - protein, 15 aa, Artificial
  Biotin
SQRIPARIHHFPTSI

SEQ ID NO: 8 - protein, 8 aa, Homo sapiens
IISAVVGI

SEQ ID NO: 9 - protein, 16 aa, Homo sapiens
QRASPLTSIISAVVGI

SEQ ID NO: 10 - protein, 18 aa, Homo sapiens
TAPLQPEQLQVFETLEEI

SEQ ID NO: 11 - protein, 80 aa, Homo sapiens
HGVTSAPDTRPAPGSTAPPAHGVTSAPDTRPAPGSTAPPAHGVTSAPDTRPAPGSTAPPAHGVTSAPDTRPAPGSTAPPA

SEQ ID NO: 12 - protein, 14 aa, Homo sapiens
KAAELIPLHKLAAK

SEQ ID NO: 13 - protein, 14 aa, Artificial
  Biotin linked to C-terminal
SGSGHGVTSAPDTR

SEQ ID NO: 14 - protein, 20 aa, Artificial
  Biotin linked to C-terminal
DDLMLSPDDIEQWFTEDPGP

SEQ ID NO: 15 - protein, 14 aa, Artificial
  Biotin linked to C-terminal
PPTGEEDTAEKDEL

SEQ ID NO: 16 - protein, 15 aa, Homo sapiens
TVETPQTAKTAYYAT

(19) United States
(12) Patent Application Publication
(10) Pub. No.: US 2012/0202226 A1
(43) Pub. Date: Aug. 9, 2012

(54) METHOD AND SYSTEM FOR THE DETECTION OF CANCER

(75) Inventors: Galit Yahalom, Modiin (IL); Alon Hayka, Judean Hills (IL)

(73) Assignee: Eventus Diagnostics Israel Ltd., Judean Hills (IL)

(21) Appl. No.: 13/380,408

(22) PCT Filed: Jun. 23, 2010
(86) PCT No.: PCT/IL2010/000498

§ 371 Date: Apr. 19, 2012

Related U.S. Application Data
(60) Provisional application No. 61/219,539, filed on Jun. 23, 2009.

(51) Int. Cl. G01N 33/566

(52) U.S. Cl. 435/7.92; 436/501

(57) ABSTRACT

Disclosed are a method and kit of diagnosis of cancer in a body sample of a subject, comprising contacting the sample with at least two different suitable antigens to form at least two different complexes with antibodies present in the sample, determining the actual levels of each of said antigen-antibody complexes in said sample and establishing the ratio between the levels of the different complexes in said subject; and comparing the ratio to a predetermined ratio between antigen-antibody complexes levels formed between the same at least two antigens and samples from healthy subjects, whereby if said ratio determined in step higher or lower than a predetermined cutoff point pre-established for healthy subjects, said subject is diagnosed with cancer. The method and kit can be used for diagnosing various types of cancer, including breast, ovary, lung, prostate and colon cancer.

### FIELD OF THE INVENTION

The present invention relates to the field of cancer diagnostics. More specifically, the present invention relates to the diagnosis of cancer based on specific antigen/antibodies complexes.

### BACKGROUND OF THE INVENTION

All publications mentioned throughout this application are fully incorporated herein by reference, including all references cited therein.

Various immunoassay methods for the detection of cancer in body samples of a subject have been developed. A part of these methods is based on the detection the presence of autoantibodies that are considered to be associated with cancer cells.

Cancer or tumor cells emerge from normal cells in the body (both human and any other animals known to have tumors), which undergo changes and become tumorigenic. While these changes start off as mutations in the genetic code of the cells, they translate into changes in protein content and/or protein expression levels, triggering changes in the behavior of the cells.

Tumor cells are antigenically different from normal cells for the presence of “tumor antigens”. These may be unique to the tumor cell, or might be expressed differently or in excess amounts and thus are considered “tumor-associated antigens” (TAAs). Tumor cells may differ from normal cells not only in protein primary structure, i.e. amino acid sequence (derived from changes in the genomic sequence), but also in the secondary and tertiary structures, due to changes in post-translational modifications, such as changes in glycosylation, phosphorylation, which consequently change the antigenicity of said proteins, also characterizing the same as tumor-associated antigens. One typical example is the protein mucin from the mammary glands, which itself does not change in the tumor cell, yet autoantibodies are found against it in patients with breast cancer (and sometimes ovarian cancer too). The reason is probably that the protein in normal cells is highly glycosylated and not exposed at all due to the dense and thick coat of carbohydrate chains. In tumor cells glycosylation is poor, leading to exposure of protein fragments, which serve as antigenic determinants to the immune system.

Another general example of a normal protein appearing and serving as a new antigen for the immune system is that of normal proteins appearing in new context, such as embryonic proteins being expressed “de nove” in adult cells.

Without being bound to any definition, TAA is currently considered to be a molecule that may be associated with specific tumors, for example lymphomas, carcinomas, sarcomas or melanomas, that may elicit cellular and/or humoral immune responses against the tumor, but rarely defends the host against the tumor. Thus TAAs are currently divided into 3 classes: highly specific for a particular tumor, present in one or only a few individuals and not found in normal cells, e.g. tumor-specific transplantation antigen (Class 1); present in a number of related tumors from different patients (Class 2); and present on normal and malignant cells, but expressed in high amounts in malignant cells (Class 3). Class 2 TAAs are considered to have the greatest potential for clinically useful assays, as they are present in many tumors and are rarely observed in normal subjects.

Some works refer to the identification of more than one autoantibody, in order to increase the sensitivity of the test [e.g. Zhang J. Y., et al., Cancer Epidemiology & Prevention 12:136-143 (2003)] Other studies referred to “antibody profiling” to describe arrays of antibodies whose presence is expected to distinguish between healthy subjects and cancer subjects [e.g. Chen, G., et al., Cancer Res. 67(7)3461-3467 (2007); Zhong et al., Journal of Thoracic Oncology 1(6) pp. 513-518 (2006)].

WO2008/008708 discloses method for detecting a presence of lung cancer in a subject comprising the steps of providing a sample from the subject; and analyzing the sample for presence of at least two markers associated with lung cancer; wherein lung cancer may be present in said subject if at least half of said markers are present in the sample; or lung cancer may be present in said subject if on obtaining a normalized value correlated with presence of each of said at least two markers in said sample, aggregating said normalized values to yield a sum; and comparing said sum to a reference value which is the maximal predictive value of lung cancer of said at least two markers, said sum is at least 30% of said reference value.

Uses and objects of the invention will become apparent as the description proceeds.

### SUMMARY OF THE INVENTION

Unlike methods of detecting presence of cancer markers or panel of markers (such as autoantibodies), the present invention provides for a diagnostic method and application that on one hand analyzes body samples for relative and quantitative measurements of the specific antibody-antigen complex levels for plurality of antigens, the measurement values being adjusted according to a predetermined contribution factors such that antigens are accounted for the physiological or otherwise contribution to the occurrence of cancer in the diagnosed subject, and on the other hand, accounts for the diverse nature of antibody expression profiles/levels across a population. The diagnostic method and application of the present invention accounts for the relative pair-wise levels of autoantibodies levels in a body sample of the diagnosed subject, and also provides technical solution to various limitations of assay devices.

An embodiment of the invention relates to a method of assigning a diagnosis to a subject being assessed for the presence of cancer and/or determining that a subject has an increased likelihood of being afflicted, the method comprising (i) providing a body sample from said subject; (ii) contacting said sample with a predetermined set of antigens to form complexes with autoantibodies present in said sample, said autoantibodies being capable of specifically binding to said antigens; wherein each of said antigens is characterized by a predetermined relative contribution factor to the presence of cancer; (iii) measuring the levels of each of said antigen-antibody complexes in said subject; (iv) determining the relative contribution parameters of each of said antigen-antibody complexes levels to the presence of cancer by adjusting each of said antigen-antibody complexes levels in accordance with the predetermined relative contribution factor; (v) and determining the output of a test function, (x)=f(relative contribution parameters); whereby if said (x) is higher than a threshold pre-established for healthy subjects, said subject is assigned with a diagnosis of an increased likelihood of currently being afflicted with cancer.

In all embodiments of the method of the invention, the set of antigens can comprise at least two antigens, each of said antigens being characterized by a predetermined relative contribution factor to the presence of cancer in said subject; the predetermined relative contribution factors defining a relative contribution factor matrix.

In all embodiments of the method of the invention, the relative contribution factor matrix comprises the proportional relationship of two or more antigen-antibody complexes levels characterizing the occurrence of cancer in said diagnosed subject.

In all embodiments of the method of the invention, the body sample may be, but is not limited to, a plasma or serum sample.

In all embodiments of the method of the invention, the sample may be divided into aliquots, for example a first aliquot which may be diluted with a suitable buffer solution at a first dilution rate, ranging, for example, at from 1:5 to 1:2000, to provide measurable antigen-antibody complexes levels; and a second aliquot, which may be diluted with a suitable buffer solution at a second dilution rate, ranging, for example, at from 1:5 to 1:2000, to provide measurable antigen-antibody complexes levels. The second dilution rate may be different from the first dilution rate; the first and second dilution rates are such that the autoantibodies are diluted to levels which following contact with the antigens provide measurable antigen-antibody complexes levels, at different dilution rates, the antigen-antibody complexes being with two different antigens.

In all embodiments of the invention, the first and second dilution rates define a relative dilution ratio of said two different antigens, or a proportional relationship between said two different antigens. The said relative dilution ratio may comprise at least two relative dilution ratios.

In embodiments of the invention, the method is designed for detecting and assigning a diagnosis of breast or ovarian cancer, cervical cancer, colon, lung or prostate cancer, but is not limited thereto.

In a further embodiment the invention provides a diagnostic monitoring system for use in assigning a diagnosis to a diagnosed subject being assessed for the presence of cancer, the system comprising: (i) a register for maintaining a relative contribution factor matrix; the relative contribution factor matrix comprising at least two predetermined relative contribution factors; (ii) an input module for receiving measured data comprising antigen-autoantibody complexes levels being obtained by contacting a body sample of said diagnosed subject with a predetermined set of antigens to form complexes with autoantibodies of said sample; wherein each of said antigens is characterized by said predetermined relative contribution factor to the presence of cancer; (iii) a processor module for processing said measured data and said relative contribution factor matrix; said processing comprising determining relative contribution parameters of said antigen-autoantibody complexes levels by adjusting each of said antigen-autoantibody complexes levels in accordance with the predetermined relative contribution factor; and determining the output (x) of a test function (x)=f(relative contribution parameters); whereby if said (x) is higher than a threshold pre-established for healthy subjects, a system variable indicates that the diagnosed subject is assigned with a status according to which the diagnosed subject is inflicted with cancer; and (iv) an output unit for outputting an indication stored in said system variable that the diagnosed subject is assigned with a status according to which the diagnosed subject is inflicted with cancer.

In another embodiment, the invention relates to a computer-implemented diagnostic method for use in assigning a diagnosis to a diagnosed subject being assessed for the presence of cancer, comprising: (i) obtaining a relative contribution factor matrix; the relative contribution factor matrix comprises at least two predetermined relative contribution factors; (ii) receiving measured data comprising antigen-antibody complexes levels being obtained by contacting a body sample of said diagnosed subject with a predetermined set of antigens to form complexes with autoantibodies of said sample; wherein each of said antigens is characterized by said predetermined relative contribution factor to the presence of cancer; (iii) processing said measured data and said relative contribution factor matrix; said processing comprising determining relative contribution parameters of said antigen-autoantibody complexes levels by adjusting each of said antigen-autoantibody complexes levels in accordance with the predetermined relative contribution factor; and determining the output (x) of a test function (x)=f(relative contribution parameters); (iv) comparing said output (x) with a threshold pre-established for healthy subjects, whereby if said (x) is higher than said threshold, said system variable is assigned with a status according to which the diagnosed patient is inflicted with cancer; and (v) outputting an indication that the diagnosed subject is assigned with the status according to which the diagnosed subject is inflicted with cancer.

In yet another embodiment, the invention relates to a computer program product for assigning a diagnosis to a diagnosed subject being assessed for the presence of cancer, the computer program product comprising a computer readable medium having a computer program code stored therein that, when executed by a processor, causes the said computer-implemented method to be performed.

In another embodiment, the invention relates to method for encoding an antigen index, comprising: (i) obtaining information comprising a set of antigens being used to form complexes with autoantibody present in a body sample; (ii) for each of the antigens; obtaining information indicative of a dilution rate such that using a suitable buffer solution at the dilution rate provides measurable antigen-autoantibody complexes levels in an assay which comprises contacting the sample with a predetermined set of antigens to form complexes with autoantibodies present in the sample, the autoantibodies being capable of specifically binding to the antigens; and (iii) encoding the antigen index; wherein the antigen index manages information indicative of the dilution rate; wherein the antigen index comprises keys and associated values; wherein each key maintains the identity of a candidate antigen, wherein each value maintains the information indicative of dilution rate for the candidate antigen; whereby in response to a query comprising a antigen of interest; the index retrieves the information indicative of the dilution rate for the antigen of interest. In some embodiments, the index maintains information indicative of the dilution rate of at least two antigens.

In a further embodiment the invention relates to a computer program product for encoding an antigen index, the computer program product comprising a computer readable medium having a computer program code stored therein that, when executed by a processor, causes the said computer-implemented diagnostic method to be performed.

In all embodiments, the matrix can by an array of values maintaining at least two relative contribution factors. e.g., [b₀, b₁, . . . bₙ], as further explained below.

Still further, the invention provides for a kit for the diagnosis of cancer in a human subject, said kit comprising: (a) buffer solution for optionally diluting a body sample from a diagnosed subject; (b) at least two antigens, wherein each of said antigens is characterized by a predetermined relative contribution factor to the presence of cancer; said predetermined relative contribution factors defining a relative contribution factor matrix being maintained in a register; and (c) reagents and means for measuring antigens-autoantibodies complexes specific for said antigens in a body sample from a subject; and (d) instructions for use.

The kit of the invention may comprise any of the computer program products in accordance with the invention.

The kit of the invention may comprise a processor module for processing measured data comprising antigen-antibody complexes levels being obtained by contacting the body sample of said diagnosed subject with said antigens to form complexes with autoantibodies of said sample; said processing comprises determining relative contribution parameters of said antigen-antibody complexes levels by adjusting each of said antigen-antibody complexes levels in accordance with the predetermined relative contribution factor; and determining the output of a test function (x)=f(relative contribution parameters); wherein a value of said (x) being higher than a threshold pre-established for healthy subjects, indicates that the diagnosed subject is inflicted with cancer.

The kit of the invention may be designed for testing body sample such as plasma or serum samples.

The antigens comprised in the kit of the invention may be at least two antigens selected from the antigens denoted by SEQ. ID. NOs. 1 to 26.

In a yet further embodiment, the invention relates to method for determining a predicted optical density (OD) reading of antibody-antigen complexes at a dilution rate of interest in an assay; the assay being performed with an assay device by providing a biological (or body) sample and contacting the sample with an antigen specie to form complexes with antibodies present in the sample, the antibodies being capable of specifically binding to the antigen specie; characterized in that the predictive optical density (OD) is determined by: (i) obtaining at least 3 OD measurements of the antibody-antigen complexes at least 3 different dilution rates; thereby obtaining data comprising at least 3 pairs of dilution rate and an assigned OD measurement.; (ii) determining a function [OD]=f (dilution rate) by a statistical smoothing procedure; and (iii) determining the [OD] value for f(the dilution rate of interest); thereby obtaining the predicted optical density (OD) reading of antibody-antigen complexes at a dilution rate of interest.

The function [OD]=f(dilution rate), if inputted with one of the at least 3 different dilution rates, the function [OD] outputs the assigned measured OD (or outputs a value of about the value of the assigned measured OD).

This method may further comprise the step of verifying that all said at least 3 OD measurements are within a linear range of the assay device. The predicted optical density (OD) reading can be outside the linear range of the measuring device.

In all embodiments, the body sample can be obtained from a mammal or a human subject.

An embodiment of the invention relates to a method of diagnosis of cancer in a subject, said method comprising the steps of (a) providing a body sample from said subject; (b) contacting said sample with at least two different suitable antigens to form at least two different complexes with antibodies present in said sample, said antibodies being capable of specifically binding to said antigens, wherein each of said antigens is characterized by the feature that the ratio between the antibody levels specific for said at least two antigens in samples from subjects who have cancer differs from the ratio between the antibody levels specific for the same at least two antigens established for healthy subjects; (c) determining the actual levels of each of said antigen-antibody complexes in said subject; (d) establishing the ratio between the levels of said at least two different complexes in said subject; and (e) comparing said ratio to a predetermined ratio between antigen-antibody complexes levels formed between the same at least two antigens and samples from healthy subjects, whereby if said ratio determined in step (d) is higher or lower than a predetermined cutoff point pre-established for healthy subjects, said subject is diagnosed with cancer. In such method, the predetermined cutoff point pre-established for healthy subjects may be an upper limit or a lower limit of a range of ratios between antigen-antibody complexes levels formed between the said same at least two antigens pre-established for healthy patients.

In all embodiments of the invention, the sample or an aliquot thereof can be diluted with a suitable buffer solution to a dilution that provides a suitable detectable level of antibodies, for example, but not limited to, a dilution of from 1:5 to 1:2000.

In all embodiments of the invention, the said antigens may be any one of the antigens denoted by SEQ. ID. Numbers 1 to 26.

In all embodiments, the method of the invention can be designed for specific, predetermined specificity and/or sensitivity, which can be adapted to the type of cancer to be detected.

In an embodiment of the invention, it provides a method for establishing specific antigen pairs, efficient for detecting specific types of cancer. This method may employ receiver operating characteristic (ROC) analysis of known cancer samples, as well as of samples from healthy subject. Special algorithms for establishing such “good” pairs are provided.

In an embodiment of the invention, there is provided a method for determining a cutoff ratio or range of ratios for diagnosing cancer for specific pairs of antigens used in the diagnostic method of the invention, which cutoff ratio or range of ratios may be designed in accordance with specific requirements of sensitivity and specificity for each type of cancer.

In some embodiments, the diagnostic methods of the present invention are performed using a set of antigens comprising at least two antigens selected from the group consisting of SEQ. ID NO. 5 (LDPe071), SEQ. ID NO. 6 (LDPe070), SEQ. ID NO. 7 (LDPe069), SEQ. ID NO. 9 (LDPe002), SEQ. ID NO. 10 (LDPe008), SEQ. ID NO. 11 (LDPe012), SEQ. ID NO. 12 (LDPe016), SEQ. ID NO. 13 (LDPe039), SEQ. ID NO. 21 (LDPe041), SEQ. ID NO. 14 (LDPe066), SEQ. ID NO. 15 (LDPe072), SEQ. ID NO. 22 (LDPe076), SEQ. ID NO. 23 (LDPe077), SEQ. ID NO. 24 (LDPe078), SEQ. ID NO. 25 (LDPe079), and SEQ. ID NO. 26 (LDPe095); the diagnosis being of breast cancer.

In other embodiments, the diagnostic methods of the present invention are performed using a set of antigens comprising at least two antigens selected from the group consisting of SEQ. ID NO. 8 (LDPe001), SEQ. ID NO. 9 (LDPe002) and SEQ. ID NO. 16 (LDPe092); the diagnosis being of ovarian cancer.

In some embodiments, the diagnostic systems of the present invention utilize a set of antigens comprising at least two antigens selected from the group consisting of SEQ. ID NO. 5 (LDPe071), SEQ. ID NO. 6 (LDPe070), SEQ. ID NO. 7 (LDPe069), SEQ. ID NO. 9 (LDPe002), SEQ. ID NO. 10 (LDPe008), SEQ. ID NO. 11 (LDPe012), SEQ. ID NO. 12 (LDPe016), SEQ. ID NO. 13 (LDPe039), SEQ. ID NO. 21 (LDPe041), SEQ. ID NO. 14 (LDPe066), SEQ. ID NO. 15 (LDPe072), SEQ. ID NO. 22 (LDPe076), SEQ. ID NO. 23 (LDPe077), SEQ. ID NO. 24 (LDPe078), SEQ. ID NO. 25 (LDPe079), and SEQ. ID NO. 26 (LDPe095); the diagnosis being of breast cancer.

In other embodiments, the diagnostic systems of the present invention utilize a set of antigens comprising at least two antigens selected from the group consisting of SEQ. ID NO. 8 (LDPe001), SEQ. ID NO. 9 (LDPe002) and SEQ. ID NO. 16 (LDPe092); the diagnosis being of ovarian cancer.

The invention will be further described on the hand of the following Figures, which are illustrative only and do not limit the scope of the invention which is defined by the appended claims.

### BRIEF DESCRIPTION OF THE FIGURES

FIG. 1: A. An example of direct measurement of complexes at same dilutions (see Material and Methods).
B. An example of direct measurement of complexes at different dilutions that cannot be performed due to device limitations (see Material and Methods).
C. An example of direct measurement of complexes at different dilutions and mathematically overcoming device limitations (see Material and Methods).

FIG. 2A: graphs plotted for 3 dilutions of CT1 and CT2 for antibodies for antigens LDPe051, LDPe064, LDPe069 and LDPe070.

FIG. 2B: Calculated ratios between LDPe051 and LDPe069 for OC (ovarian cancer), CT (healthy control) and BC (breast cancer) (Dilution No. 2).

FIG. 2C: Table 7—calculations for ROC curve for breast cancer 51_{—}69 (Example 1).

FIG. 2D: ROC curve for breast cancer for the results presented in Example 1—antigens LDPe051 and LDPe069.

FIG. 2E: Table 8—calculations for ROC curve for ovarian cancer 51_{—}69 (Example 1).

FIG. 2F: ROC curve for antigens LDPe051 and LDPe069 in diagnosis of ovarian cancer.

FIG. 2G: Calculated ratios between LDPe064 and LDPe070 for OC (ovarian cancer), CT (healthy control) and BC (breast cancer) (Dilution No. 2).

FIG. 2H: Table 9—calculations for ROC curve for ovarian cancer 64_{—}70 (Example 1).

FIG. 2I: ROC curve for antigens LDPe064 and LDPe070 in diagnosis of ovarian cancer.

FIG. 2J:—Table 10—calculations for ROC curve for breast cancer 64_{—}70 (Example 1).

FIG. 2K: ROC curve for antigens LDPe064 and LDPe070 in diagnosis of breast cancer.

FIG. 3: Examples of analysis of results with various pairs of antigens (A: SEQ ID NOs. 18 and 20; B: SEQ ID NOs. 19 and 20; C: SEQ ID NOs. 1 and 2; D: SEQ ID NOs. 1 and 20; E: SEQ ID NOs. 2 and 5; F: SEQ ID NOs. 4 and 17; G: SEQ ID NOs. 5 and 17; H: SEQ ID NOs. 3 and 17; I: SEQ ID NOs. 5 and 19).

FIG. 4: Table 11—sequences of specific antigens.

FIGS. 5A-5D: FIG. 5A—showing antigen-autoantibody complex level measurements (test1139) obtained for 4 antigens, the measurements obtained in an identical series of dilution rates; FIG. 5B—showing smoothed measurements obtained for the 4 antigens of test1139; FIG. 5C—showing antigen-autoantibody complex levels measurements obtained for the 4 antigens (test1139), the measurements obtained in varying dilution rates for each antigen: Ag1—1:8, Ag2—1:32, Ag3—1:64, Ag4—1:512; FIG. 5D—showing smoothing results obtained for Ag 1-4 with test1139, starting dilution of sample for each antigen was for Ag1—1:8, Ag2—1:32, Ag3—1:64, Ag4—1:512.

FIG. 6A-6D: FIG. 6A—showing antigen-autoantibody complex level measurements (test1200) obtained for 4 antigens, the measurements obtained in an identical series of dilution rates; FIG. 6B—showing smoothed measurements obtained for the 4 antigens of test1200; FIG. 6C—showing antigen-autoantibody complex levels measurements obtained for the 4 antigens (test1200), the measurements obtained in varying dilution rates for each antigen: Ag1—1:8, Ag2—1:32, Ag3—1:64, Ag4—1:512; FIG. 6D showing smoothing results obtained for Ag 1-4 with test1200, starting dilution of sample for each antigen was for Ag1—1:8, Ag2—1:32, Ag3—1:64, Ag4—1:512.

FIGS. 7A-7M: FIG. 7A—provides ROC analysis determining the AUC for a subset comprising 14 antigens permitting both statistical separation between diseased/cancer subjects and healthy controls; FIG. 7B—Table 17, detailing the ln(OD) results for the first dilution rate or point of each antigen and each sample. row “0” represents healthy samples, and row “1” represents cancer sample; FIG. 7C—provides ROC analysis determining the AUC for a subset comprising 13 antigens; FIG. 7D—provides ROC analysis determining the AUC for a subset comprising 12 antigens; FIG. 7E—provides ROC analysis determining the AUC for a subset comprising 11 antigens; FIG. 7F—provides ROC analysis determining the AUC for a subset comprising 10 antigens; FIG. 7G—provides ROC analysis determining the AUC for a subset comprising 9 antigens; FIG. 7H—provides ROC analysis determining the AUC for a subset comprising 8 antigens; FIG. 7I provides ROC analysis determining the AUC for a subset comprising 7 antigens; FIG. 7J—provides ROC analysis determining the AUC for a subset comprising 6 antigens; FIG. 7K—provides ROC analysis determining the AUC for a subset comprising 5 antigens; FIG. 7L—provides ROC analysis determining the AUC for a subset comprising 4 antigens; FIG. 7M—provides ROC analysis determining the AUC for a subset comprising 3 antigens.

FIG. 7N—showing 2 dimensional graph of results obtained for the first minimal dilution (1:8) for LDPe002 and LDPe092 for 7 ovarian cancer patients and 17 healthy subjects

FIG. 7O—AUC curve for results obtained and shown in FIG. 7N, for the first minimal dilution (1:8) for LDPe002 and LDPe092 for 7 ovarian cancer patients and 17 healthy subjects

FIG. 7P—showing 2 dimensional graph of results obtained for the first minimal dilution (1:8) for LDPe001 and LDPe092 for 14 ovarian cancer patients and 14 healthy subjects.

FIG. 7Q—AUC curve for results obtained and shown in FIG. 7P for the first minimal dilution (1:8) for LDPe001 and LDPe092 for 14 ovarian cancer patients and 14 healthy subjects.

FIG. 8: Table 38—list of sequences of peptide/protein used as antigen to form complexes with autoantibodies in Examples 1-8.

FIG. 9: a flow chart of a computer implemented diagnostic method for use in assigning a diagnosis to a diagnosed subject being assessed for the presence of cancer.

FIG. 10: a schematic block diagram of a diagnostic monitoring system which operates for diagnostics of cancer.

### DETAILED DESCRIPTION OF THE INVENTION

As mentioned in the introductory part, during the cancerous process, future cancerous cells undergo changes, at the DNA, gene expression, post-transcriptional, translational and/or post-translational levels, which change their phenotype. In other words, these cells begin to express proteins which were previously not part of their “normal” repertoire, and which are thus identified as tumor-associated antigens (TAAs). TAAs may also be present in normal cells or even in cells from normal (healthy) subjects. Tumor antigens may be the result of new genetic information introduced by a virus; alteration of genetic function by carcinogens, possible through activation of a proto-oncogene, by which genetic material that is normally inactive (except possibly during embryonic development) is activated into an oncogene and becomes expressed in the cell phenotype; uncovering antigens which are normally present on normal cells or “buried” in the cell membrane, through the inability of neoplastic cells to synthesize membrane constituents (e.g., sialic acid); and release of antigens that are normally sequestered in the cell or its organelles, through the death of neoplastic cells.

One important outcome of the expression of TAAs is that they can lead to immune recognition by the body immune system. Immune recognition could eventually lead to the generation of TAA-recognizing antibodies, also called autoantibodies, which are maintained in a basal level in the body in order for the immune system to define “self” and “non-self”. Nonetheless, until today no specific autoantibodies have been identified as capable of differentiating between cancer patients and normal population using “cut off” criteria, particularly not in high levels of sensitivity and specificity. This can be attributed to the fact that both populations have serum autoantibodies against these TAAs. When cancer emerges, the production of these autoantibodies is changed. Other autoantibodies, not strictly referred to as TAAs, may also have different expression levels in cancer patients compared to healthy population.

In search for a method of diagnosing cancer which would be simple, cost effective, highly specific, and sensitive, it was found that it is not the blood presence of a specific autoantibody in a subject that would be diagnostic. Rather, inter-alia, the ratio between the levels any two or more autoantibodies found in the blood of both healthy individuals and cancer patients, is an important feature on which diagnosis of cancer patients can be based. In general, existing techniques are based on presence of autoantibody markers against TAAs in suspected cancer patients. In view of the present finding, the level of a certain autoantibody is not the parameter to be determined. Thus, contrary to cutoff comparisons, where actual autoantibody levels above a certain cutoff are indicative of a diagnosis of cancer, the inventors have determined a new method, determining the actual levels of autoantibodies against at least two antigens, e.g., TAAs, in a tested subject, calculating the ratio between the levels of these two autoantibodies, and comparing the ratio to the ratio of the actual levels of autoantibodies to the same at least two antigens in a predetermined normal reference population. Whenever the ratio in the sample of the tested subject is different from the reference ratio, that difference is indicative of the subject having cancer. It is to be noted that the difference can be either a higher or a lower ratio. The method above, takes into consideration the self-production of autoantibodies in healthy condition vs. cancerous condition of each subject. The change in the said ratio reflects this change in the population.

In order to assess the novel method of the invention, the inventors used, for experimental purposes, certain TAAs, as will be detailed below. However, it is important to note that the antigens to be used in the methods of the invention are not necessarily antigens classically defined as TAAs. An antigen of interest for the method of the present invention is an antigen that specifically binds to an autoantibody present in both the tested sample and in sample/s from healthy control/s, where the ratio between the actual levels of at least two such autoantibodies in a cancer positive patient is different from the same ratio established for healthy population. As will be shown in the Examples below, according to the present invention the mere presence of an autoantibody recognized by antigens that are known to be TAAs is not indicative that that subject has cancer, neither is the actual level of such autoantibody. Therefore, the antigens useful in the method of the invention need not necessarily be classical TAAs. Antigens, and in particular pairs of at least two antigens, suitable for use in the present invention can be identified by a person of ordinary skill in the art using the methods described herein.

Most importantly, the methods described herein are based on a blood test, and use plasma or serum samples. Consequently, the method of the invention is a faster, cheaper and overall advantageous method compared to known diagnostic methods.

As will be detailed hereafter, the tested sample and/or aliquots thereof, may be serially diluted before being contacted with the antigen/s, in order to suit the detection limits of the detection method/device. The dilution is desired, for example, when the levels of autoantibodies in the sample are high. The degree of dilution may be readily determined by the skilled technician. Generally, each sample or aliquot thereof should be serially diluted to a level needed for the proper detection of all antigen-autoantibody complexes according to the method of detection (being ELISA, FACS, Western Blot, etc.). It is not necessary that all samples be diluted to the same dilution, and it is not necessary that each complex is measured in the same dilution, as will be shown in the Examples below. Establishing a ratio between two complexes for a specific dilution determines the ratio for each subject, i.e. for subjects with high levels of autoantibodies, a higher dilution should be made, while a sample of another subject, with a lower autoantibodies level, should be diluted to a lesser extent. Since the ratio is a “self ratio”, there is no need to keep all conditions similar between all subjects. In case two antibodies cannot be detected in the same system because different dilutions are required, different dilutions may be employed, and corresponding values may be determined by extrapolation, as shown in FIG. 1 (A, B, C) and in the Examples below. Dilution may be to a suitable level, specifically a level that is suitable for the detection system used. Thus, for different detection systems that have different specifications and technical limitations, different dilutions may be required, to be above the basic detection level. The designing of particulars of the assay, such as the extent of dilution, is within the skills of the man of the art. It is important to maintain the predetermined relative dilution ratio between the diluted samples for each of the antigen complex tested for all samples.

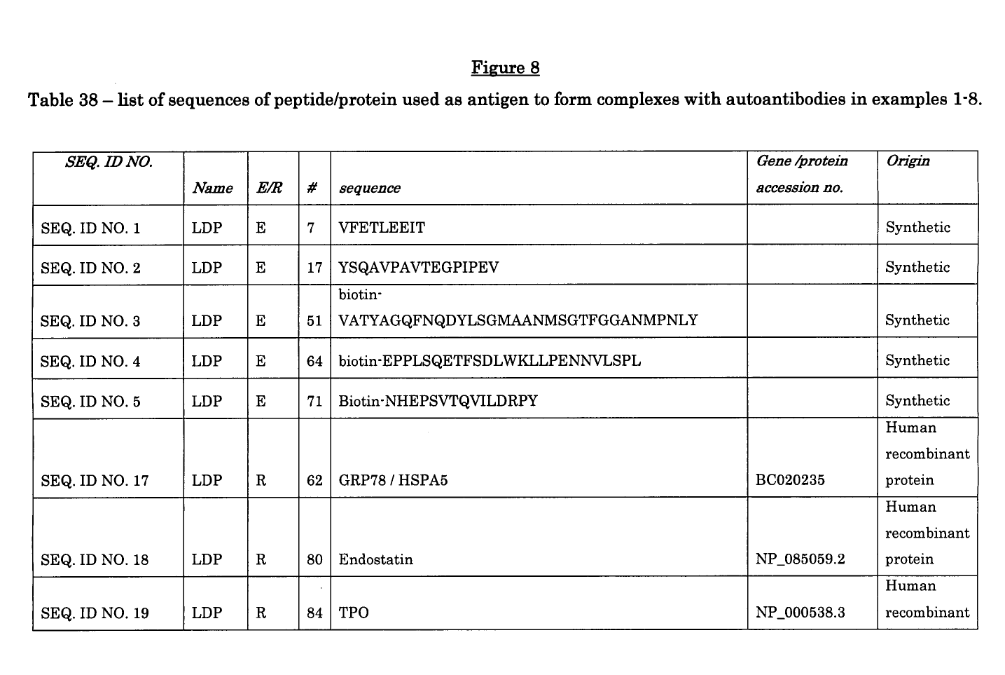

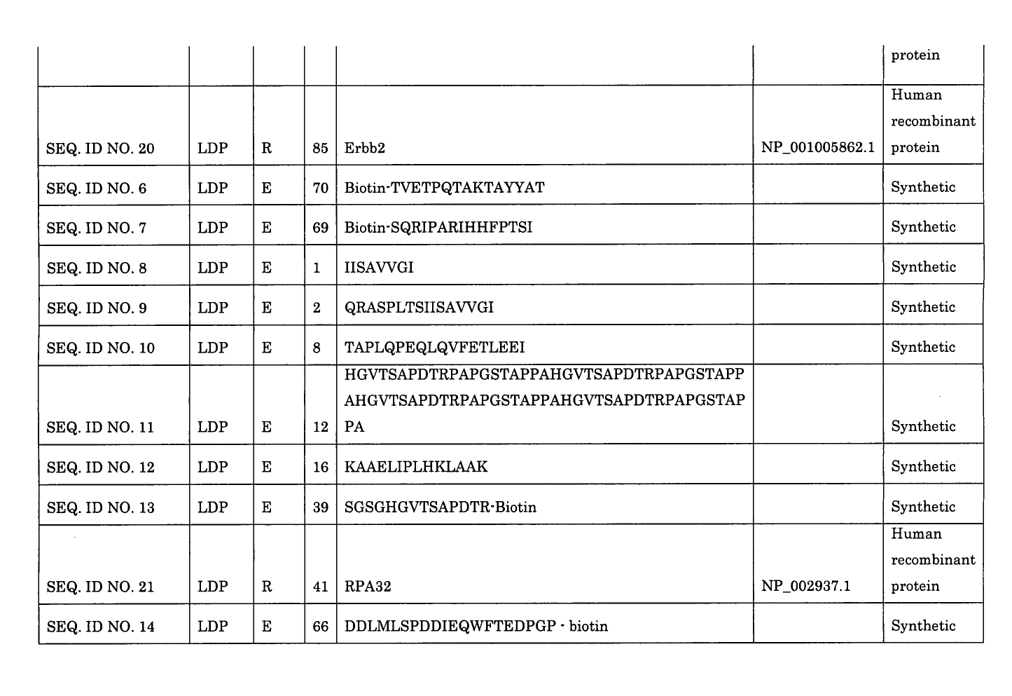

For purpose of the present study, and without being limiting, the present inventors selected a number of antigens (including peptides,), specified in Table 11 (and in FIG. 8, Table 38) and used them in the identification/detection of the corresponding antibodies in a plasma sample. As used herein, the term antigen is to be taken to mean any substance that when introduced into the body stimulates an immune response which can lead to the production of an antibody. In certain embodiments, the antigens are tumor-associated peptides of at least two amino acids and longer, peptides and proteins comprising such tumor-associated peptides and derivatives thereof. Nonetheless, as described above, antigens need not necessarily be those classically known as TAAs.

In addition to peptide-based and protein-based antigens, other antigens may be used, for example nucleic acid-based, carbohydrates-based, lipid-based, natural organic-based, synthetically-derived, organic-based, inorganic-based, and peptidomimetics-based substances. Such substance may be a product of, for example, positional scanning of combinatorial libraries of peptides, libraries of cyclic peptidomimetics, and random or dedicated phage display libraries.

The present application utilizes specific antigens (TAAs) defined by SEQ. ID. NOs. 1-26 (as shown in FIG. 8—Table 38) Essentially, TAAs and TAA-recognizing antibodies are described herein as important tools for the diagnosis of cancer.

Thus, the antigens of the invention were used per se or as the active agent comprised in a diagnostic composition for the detection of antibodies in a plasma sample obtained from a subject. Detection of specific antigen-recognizing antibodies is effected by contacting the sample with at least two specific antigens (i.e., with the antigens of the invention, for example the antigens denoted by SEQ. ID. NO. 1 to 26, and detailed in Tables 1, 5, 11 and 38 (FIG. 8)). Preferably, the antigens are used at concentrations of about 2.5-250 μg/ml (for an ELISA based assay of antibody detection).

The term “antibody” or “autoantibody” as used herein is also meant to include both intact molecules as well as fragments thereof, such as, for example, scFv, Fv, Fab′, Fab, diabody, linear antibody, F(ab′)₂ antigen binding fragment of an antibody which are capable of binding antigen [Wahl et al. (1983) J. Nucl. Med. 24, 316-325]. As defined herein “antibody” or “autoantibody” may be any one of IgG, IgM and IgA. Without being bound by theory, it is expected that autoantibodies indicative of cancer are primarily IgGs. Measuring IgG may be more specific.

An antibody is said to be “capable of binding”, or “recognizing” a molecule if it is capable of specifically reacting with the molecule and thereby binding said molecule to the antibody. The term “epitope” is meant to refer to the portion of any molecule capable of being bound by an antibody, which can also be recognized by that antibody or the cells producing that antibody. Epitopes or “antigenic determinants” usually consist of chemically active surface groupings of molecules such as amino acids or sugar side chains, and have specific three-dimensional structural characteristics as well as specific charge characteristics.

An “antigen” is a molecule or a portion of a molecule capable of being bound by an antibody. An antigen may have one or more than one epitope. The specific reaction referred to above is meant to indicate that the antigen will react, in a highly selective and specific manner, with its corresponding antibody and not with the multitude of other antibodies which may be evoked by other antigens.

The antibodies, or fragments thereof, to be detected by the present invention, may be detected in the subject's sample by any method. This can be accomplished by techniques giving a visually detectable signal, which may be any one of fluorescence (immunofluorescence), a chromogenic product of an enzymatic reaction, production of a precipitate, chemiluminescence or bioluminescence. Generally, the antigen/s is/are immobilized on a suitable support, particularly solid support, the biological sample containing the autoantibody is then contacted with the antigen's, the detection means such as enzyme, tag, colour, etc, are added and the level of the autoantibody is measured. More details may be found in the experimental section below. Other techniques which may be used for detecting the autoantibody include, but are not limited to colloidal gold, radioactive tag, GFP (green fluorescence protein), and the like, avidin/streptavidin-biotin, magnetic beads, as well as physical systems, e.g. nanotechnological system, sensitive to the actual binding.

The support can be a “solid phase support”, “solid phase carrier”, “solid support”, “solid carrier”, “support” or “carrier”, all of which are capable of binding the antigen. Well-known supports or carriers, include glass, polystyrene, polypropylene, polyethylene, dextran, nylon amylases, natural and modified celluloses, polyacrylamides, and magnetite. The nature of the carrier can be either soluble to some extent or insoluble for the purposes of the present invention. The support material may have virtually any possible structural configuration so long as the coupled/immobilized antigen molecule is capable of binding to an antibody. Thus, the support or carrier configuration may be spherical, as in a bead, cylindrical, as in the inside surface of a test tube, or the external surface of a rod. Different carriers may be used for different antigens within the same tube. Alternatively, the surface may be flat such as a sheet, test strip, etc. Preferred supports or carriers include polystyrene beads. Those skilled in the art will know many other suitable carriers for binding antigens, or will be able to ascertain the same by use of routine experimentation.

Other such steps as washing, stirring, shaking, filtering and the like may be added to the assays as is customary or necessary for the particular situation.

The present invention provides for a diagnostic method and application that on one hand analyzes a body sample for measurements of the specific antibody-antigen complexes for plurality of antigens, each having different physiological or otherwise contribution to the occurrence of cancer and on the other hand, accounts for the diverse nature of antibody expression profiles across a population. In this respect, a subject's production of autoantibodies may be characterized as “strong” having relatively substantial levels of a certain autoantibody in comparison to another subject's “weak” autoantibody production.

As demonstrated below, the present invention provides an assay that addresses these challenges inter-alia by adopting a flexible diagnostic approach. Instead of performing a diagnostic assay on a body sample (e.g. plasma or serum) being diluted at a predefined single dilution rate applicable to all antibody-antigen complexes, the present invention discloses diagnostic assays performed at plurality of dilutions rates (2, 3 and more) and teaches collection and consolidation of the information gathered there from. As shown below, a single range of dilutions for all antibody-antigen complexes for simultaneous use is not always available. Even if it were, limiting the assay to a single dilution rate, would pose a technical constraint for detecting device in the clinical settings.

Therefore, conditions arise where it is not applicable to use the same dilution rates for all the antigens either while identifying a diagnostic set of antigens or while carrying out diagnostic tests on myriad patient samples. A single dilution rate may not be applicable for use in the various clinical settings not only for reasons of inaccuracies but also because of assay device limitations. Inaccuracies may occur, for example, when one of the antibody-antigen complex formed in the sample yields a very high (or very low) and could not be measured with a specific detecting device.

In order to overcome this problem, the present invention provides sets of different dilutions rates pre-defined for different antibody-antigen complexes. In additional, relative dilution ratios between different dilutions rates, as determined for various different antigen-autoantibody complexes is defined and is always maintained for all the tested samples.

Moreover, there may be a case when for a specific sample, all antibody-antigen complexes are very high, and it is impossible to use the same initial dilution rate as in the other samples. The present invention further teaches to dilute the “high” sample to a determined representative dilution rate, from which some or all the dilution rates will be derived, for example, by maintaining the dilution ratio between dilution rates of antibody-antigen complex for different antigens. Therefore, following the obtaining of an antibody-antigen complexes level at a predetermined representative dilution rate, another antibody-antigen complexes level can be obtained at a second dilution rate maintaining the dilution ratio between the predetermined representative dilution rate and the second dilution rate. Additional antibody-antigen complexes levels can be obtained similarly in a successive manner.

For example, if a sample is diluted at a dilution rate of 1:5 for Ag1, and at a dilution rate of 1:10 for Ag2 then, the dilution ratio between Ag1 and Ag2 is 2 (10/5). This dilution ratio should be maintained in all samples measured for the same antigens (Ag1, and Ag2). For high levels of autoantibodies, the sample may be diluted to 1:100 (for Ag1) and 1:200 (for Ag2), but the dilution ratio is maintained 2 (200/100). This is one of the reasons as to why the actual presence of antibody will not be sufficient to determine diagnosis, as for “high” samples, antibody will always be present.

Instead of using repeated multiple OD measurements (such as duplicate, triplicates etc.) to merely increase reliably of the readings, OD measurement were obtained in specific range of dilution rates for each antigen by utilizing serial dilution procedures performed for each of the antibody-antigen complexes. In this way, for each of the antibody-antigen complex a representative dilution rate is selected. A representative dilution rate is one that which produces an OD measurement at the linear range of the measuring device. For example, the representative dilution rate of an antigen may be the first dilution rate used to obtain an OD reading in the series of measurements and which produces results within the linear range of the measuring device. In this manner, accurate measurement of the antibody-antigen amount of complex is obtained (i.e. within the linear range of the measuring device).

For body samples in which a diagnostic assay at a set of dilution rates produced a signal which is excessively high (exceeding the linear range of the measuring device) a predicted OD signal can be calculated under the assumption that the linear range of the device is wider, using extrapolation procedure defined herein (see, e.g., FIG. 1C). Generally, extrapolation of the present invention is performed by utilizing a function which ties an OD reading to dilution rates which can produce these readings. As previously stated, for each antigen in the diagnostic set as series of OD readings is produced as a function of a specific dilution rate and obtaining a dilution curve thereby. The data is converted (or smoothed) into the dilution function i.e. [OD]=f(dilution rate). Optionally, f may be linear, exponential, a polynomial function or the like. Where the measuring device cannot obtain signals at a particular dilution rate or obtains. OD signal outside its linear range, the function is used to extrapolate a predicted optical density (OD) reading from the OD measurements performed in other dilution ranges, preferably at the linear range of measuring device. These theoretical or predicted values are then used further for determining (or identifying) a diagnostic set of antigens, and the relative contribution factors characterizing each antigens (see Examples 5-7 below).

In order to achieve reliable results, the smoothing procedure eliminates outliers (extreme values by mathematical considerations) from the data set, and then calculate the theoretical dilution function as described above. Smoothing procedure can take the form of obtaining the function by linear regression. Using this dilution function further processing is performed to identify those antigens that will best differentiate between the cancerous and healthy population by their unique relative profile.

The following terms are used herein:

“assigning a diagnosis” shall mean providing an indication according to which a diagnosed subject is being afflicted with cancer or otherwise having an increased likelihood of being inflicted with cancer at the time the body sample was obtained.

“relative contribution factor” shall mean a variable which characterizes an antigen or being assigned to an antigen; the variable maintaining a value which represents a contribution measurement of the antigen to the presence of cancer of in a diagnosed subject. In particular, the relative contribution factor further characterizes the relative contribution of an antigen-autoantibody complexes level(s) measured in a diagnosed subject to the occurrence or presence of cancer in said subject. In particular, relative contribution factor can characterize the relative contribution of an antigen-autoantibody complexes level(s) measured at a predetermined dilution or predetermined dilution rate. In this respect, antigen-autoantibody complexes level(s) encompass either actual measurement, or smoothed/predicted measurement. By way of non-limiting illustration, the size relationship between a pair of relative contribution factors (characterizing a pair of antigens), provides the relative contribution of each of said pair to the occurrence of cancer.

A “dilution”, “dilution rate” or “dilution point” shall refer to an assay in which a body samples (such as e.g., serum or plasma) is being diluted with a suitable buffer solution; dilution rate or dilution point means decreasing the concentration of the body samples such the decreased concentration is defined by a volumetric quantity of body sample to the volumetric quantity of the suitable buffer solution. By way of non-limiting examples, the body sample can be diluted at a dilution rate of 1:8 i.e., one unit the body sample per 8 units of the buffer solution, 1:32 or the like.

“pair-wise dilution ratio” or “relative pair-wise dilution ratio” or “relative dilution ratio” shall refer to an assay including a contacting step in which a body sample is contacted with a first antigen and a second antigen of a pair of antigens, so as to form complexes with autoantibodies present in the body samples; a first aliquot of the sample being diluted at a first dilution rate (e.g., x₁), second aliquot of the sample being diluted at a second dilution rate (e.g., x₂) with a suitable buffer solution to provide measurable antigen-antibody complexes levels; a measurement or value preset for a diagnosed population and which estimates the proportional relationship between a pair of dilution rates determined for the pair of antigens; each dilution rate produced in the linear range of the measuring device. By way of non-limiting example, let x₁ and x₂ be a pair of dilution rates of Ag₁ and Ag2 respectively; the pair of dilution rates producing a pair of OD readings within the linear range of measuring device. Therefore, the relative dilution ratio can be defined as x₂/x₁, ln(x₁)/ln(x₂), etc'. Unless otherwise stated the relative dilution ratio shall mean x₁/x₂. By way of non-limiting example, where the first aliquot was obtained at a dilution rate of 1:8 i.e., x₁=1:8 (per Ag₁), and the second aliquot was obtained at a dilution rate of 1:32, i.e., x₂=1:32 (per Ag₂), the pair-wise dilution ratio is x₂/x₁=0.25.

In some embodiments, following the determination or pre-setting of a pair-wise dilution ratio as between a pair of antigens, the pair-wise dilution ratio can be maintained during operation of the systems and methods of the present invention, as exemplified below. For example, if the pair-wise dilution ratio is x₂/x₁=0.25, and an obtained antigen-autoantibody complex level per Ag₁ was 1:16, maintaining the pair-wise dilution ratio means obtaining the antigen-autoantibody complex level per Ag₂ at the dilution rate of 1:64. As explained below, if at 1:64 the measuring device is outside its linear range, a predicted is ascertained.

A “register” shall mean a record maintained by a memory device, memory utility or a part thereof. The register can be a part of computer system or otherwise computer memory. In the context of the present invention the register maintains the relative contribution factor matrix or array but may also comprises other information.

An “index” shall mean a database or any other system or utility permitting storage and retrieval of information comprising any associative data structure, array, container, dictionary which allows query-processing therewith. An index typically comprises a collection of keys and a collection of values, where each key is associated with one more value. The operation of finding the value associated with a key is commonly referred to a lookup, and this is an operation supported by the index disclosed herein.

An “antigen index” is an index comprising a collection of antigens represented by keys, each key (or antigen associated thereby) is associated with a value representing information indicative of a dilution rate for the antigen or a pair-wise dilution ratio with respect to a second antigen.

“encoding” shall mean transforming one representation into a different, equivalent representation. For example, a p53 antigen can be represented by the string “LDPr077” is a form of encoding.

A “query” shall mean a search for information in an index or database. The information can be information indicative of a predetermined dilution rate or point for a particular antigen.

Further, all numerical values (input, output, output or functions in accordance to a formula provided herein, OD measurements etc.) are approximations which are varied (+) or (−) by up to 10%, at times by up to 5% from the stated values. It is to be understood, even if not always explicitly stated that all numerical designations are preceded by the term “about”.

One of the ways in which an antigen-autoantibody complex level can be measured in accordance with the present invention is by linking enzyme immunoassay (EIA). Enzymes which can be used in the assay include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholine-esterase. The detection can be accomplished by colorimetric methods which employ a chromogenic substrate for the enzyme. Measurement may also be accomplished by visual comparison of the extent of enzymatic reaction of a substrate with similarly prepared standards (this procedure is suitable for both soluble color products and non-soluble color products, e.g. on nitrocellulose or plastic supports).

Detecting the reaction of the antibody with the antigen can be further aided, in appropriate instances, by the use of a secondary antibody or other ligand which is reactive, either specifically with a different epitope, or non-specifically with the ligand or reacted antibody.

Enzyme immunoassays such as immunofluorescence assays (IFA), photometric assays, enzyme linked immunoabsorbent assays (ELISA), ELISPOT assay, and immunoblotting can be readily adapted to accomplish the detection of the specific antibodies. An ELISA method effective for the detection of the autoantibodies can, for example, have the following steps: (1) binding the antigen of interest, e.g., a TAA, to a solid support; (2) contacting the bound antigen with a biological sample, such as plasma or serum; (3) contacting the above with a secondary antibody bound to a detectable moiety (e.g., horseradish peroxidase enzyme or alkaline phosphatase enzyme)—the secondary antibody being capable of detecting the said antibody of step (2); (4) contacting the above with the substrate for the enzyme; (5) contacting the above with a suitable reagent; (6) observing and quantifying the color change.

Other methods of immunoenzymatic detection of the presence of the antibody are the Western blot, and dot blot. The antigens are transferred to a nitrocellulose membrane or other suitable support. The sample to be tested, usually plasma, is then brought into contact with the membrane and the presence of the immune complexes formed is detected by the method already described. In a variation on this method, purified antigens are applied in lines or spots on a membrane and allowed to bind. The membrane is subsequently brought into contact with the plasma sample to be tested and the immune complexes formed are detected using the techniques described herein.

Measurement for specific antibodies in the sample may also be performed by agglutination. Antigens may be used to coat, for example, latex particles which form a uniform suspension. When mixed with plasma containing specific autoantibodies to the affixed antigens, the latex particles agglutinate and the presence of large aggregates can be detected visually.

The autoantibodies can also be measured by a variety of immunoassay methods. For a review of immunological and immunoassay procedures applicable to the measurement of antibodies by immunoassay techniques, see Basic and Clinical Immunology [D. Stites et al. (eds.), (1994) Basic and Clinical Immunology, 8^{th} Ed.].

Measuring reaction of the antibody with the antigen can be facilitated by the use of an antibody or ligand that is labeled with a detectable moiety by methods known in the art. Such a detectable moiety allows visual detection of a precipitate or a color change, visual detection by microscopy, or automated detection by spectrometry or radiometric measurement or the like. Examples of detectable moieties include fluorescein and rhodamine (for fluorescence microscopy), horseradish peroxidase and alkaline phosphatase (for either light microscopy or electron microscopy and biochemical detection and for biochemical detection by color change), and biotin-streptavidin (for light or electron microscopy). The detection methods and moieties used can be selected, for example, from the list above or other suitable examples by the standard criteria applied to such selections [Harlow and Lane (1988) Antibodies: A Laboratory Manual, Cold Spring Harbor Laboratory, Cold Spring Harbor, N.Y.].

Measurement may be accomplished using any of a variety of other immunoassays. For example, by radioactive labeling the antigens, it is possible to detect antibodies through the use of a radioimmunoassay (RIA). A description of RIA may be found in Laboratory Techniques and Biochemistry in Molecular Biology, by Work, T. S. et al, North Holland Publishing Company, NY (1978) with particular reference to the chapter entitled “An Introduction to Radioimmune Assay and Related Techniques” by Chard, T., incorporated by reference herein. The radioactive isotope can be detected by such means as the use of a gamma/beta counter or a scintillation counter or by autoradiography.

As mentioned, the sample may be serially diluted before being contacted with the antigens. In some embodiments, blocking the antigens for nonspecific binding may be recommended, for example with skim milk.

Interestingly, the ratio between the actual levels of autoantibodies present in a blood of cancer patients against at least two suitable antigens as defined herein, for example TAAs, is different from the ratio between autoantibody levels against the same at least two antigens determined for healthy individuals, also referred to herein as the reference ratio. Thus, the finding of antigen-antibody complexes in a sample must be first analyzed with a view to other antigen-antibody complexes in the subject, and the overall result compared to the pattern obtained for the same antigen-antibody complexes in the healthy, or control, disease-free population (also referred to as normal).

When measuring more than two autoantibodies, a relative contribution factor is determined for each antigen. The relative contribution factor characterizes each antigen; it maintains a value which represents a contribution measurement of the antigen to the presence of cancer of in a diagnosed subject., and embeds the relative dilution rates used for each antigen, and the relative amount of each antibody relative to all other antibodies for other antigens.

In the above-described embodiment, the ratio between two autoantibodies can be determined. However, it is also possible to establish the ratio between a specific antigen-recognizing autoantibody and a mixture of non-specific antibodies, including total Ig.

While the method can be for measuring autoantibodies in a sample of a subject, it is contemplated for the diagnosis of cancer per se. The detection of the said “different” ratio between different autoantibodies in the sample (different from the same ratio in healthy individuals) is an indication of the presence of cancer.

It is to be understood that within the scope of this invention, when the ratio between two autoantibodies in a sample is expressed by a number, that number is a limit of a range the signifies healthy subjects, or cancer patients, and the range may be the upper limit of the range, or the lower limit of the range. For example, if the ratio for healthy patients is an upper limit, all patients with a lower ratio are healthy. If the ratio is a lower limit, all patients with a higher ratio are healthy. The same applies mutatis mutandis for ratios of cancer patients. The lower or upper limits of the range can be varied, in accordance with specific requirements of the attending physician, as discussed in more detail below, and setting the limits is within the scope of this invention.

In certain embodiments, the (1) quantitative ratio between two antibodies in the sample may be determined in relation to (2) an identical ratio between the same two antibodies present in a sample obtained from a healthy or normal individual, that serves as a reference ratio or baseline ratio, to give a relative value. The relative value can thus be the result of dividing the sample ratio and the reference ratio by each other (to give a value greater than, equal to or smaller than 1, where any value other than 1 indicates that the tested sample was positive; it is within the scope of this application that relative value is substantially other than 1, i.e. determined according to parameters of the analysis employed). The relative value can be obtained also by deducting the sample ratio from the reference ratio or the reference ratio from the sample ratio (to give a positive or negative value, or zero, where any value other than zero indicates that the subject has cancer—this is shown in the following examples; it is within the scope of this application that relative value is substantially other than zero, and may be different from zero, i.e. determined according to parameters of the analysis employed). A healthy or normal subject as used herein is to mean, but is not limited to, a subject without cancer, tumor, malignancy or proliferative disorder.

The diagnostic method of the invention may be performed in various modes with respect to measuring various antigen-autoantibody complexes in one sample. For example, in one embodiment the sample may be divided into at least two aliquots, the number of aliquots being equivalent to the number of suitable antigens, as herein defined, for which antigen-recognizing antibodies are to be searched. Thus, if three antigens should be tested, the sample may be divided into three aliquots, if four antigens should be tested, the sample may be divided into four aliquots, and so forth. The sample may be divided into aliquots if such division is required for the specific immunoassay performed. If divided, subsequent steps of the method may then be performed in each aliquot in parallel, and the results of antibody-antigen complexes obtained for each sample compared and analyzed by pair-wise analysis or any other suitable statistical analysis.

The result/s obtained for the sample of the subject can then compared with the results obtained or available for the normal (healthy) population. If the result of the tested sample is different from the value established for the normal population, said subject has a positive diagnosis for cancer.

Alternatively, the diagnostic method of the invention may be performed at a high throughput scale, using for example layered peptide arrays, as described by Gannot et al. [Gannot et al. (2005) Layered Peptide Array—High-Throughput antibody screening of clinical samples. J. Mol. Diagn. Vol. 7:427-436], which would allow testing for a large number of antigen-recognizing antibodies in one sample, and even in several samples simultaneously.

Another option would be to use multiplex immunoassays. Different antigens to be tested could be labeled with various different labelings, for example different colors or different fluorescent dyes, which are detected at different wavelengths. Following the step of incubating the sample with the different antigens, the sample is analyzed for the presence of the different complexes. Quantification of the complexes may be either by ELISA, when the antigens are presented bound to a solid phase, the plate, or by FACS analysis, when the antigens are presented in solution. Standard protocols for ELISA and FACS are described herein and are well known to persons of ordinary skill in the art.

Antigens that are needed for the detection of tumor indicative autoantibodies can be in several forms, including the whole cell (of a tumor), which can serve as the antigen; cellular membranes the product of which can be used as antigens; tumor-associated proteins (or fragments thereof) isolated or recombinantly produced (which are usually prepared from constructs inserted in vectors to transform cells, bacteria, yeast, phage), or synthetically produced. Specific examples of suitable tumor antigens are the antigens detailed herein in Tables 1, 5, 11, and FIG. 8.

As cited herein, the terms tumor-specific proteins, tumor-specific antigens, tumor antigens, tumor-associated antigens, and variations thereof are used interchangeably.

The basic computation method underlying the analysis of results of the detection tests of the method of the present invention is Receiver Operating Characteristic (ROC) [http://www.medcalc.be/manual/roc.php]. By this method, the diagnostic performance of a test, or the accuracy of a test to discriminate diseased cases from normal cases is evaluated using (ROC) curve analysis [Metz C E (1978) Seminars in Nuclear Medicine, 8, 283-298; Zweig M H & Campbell G (1993) Clin. Chem. 39, 561-577]. ROC curves can also be used to compare the diagnostic performance of two or more laboratory or diagnostic tests [Griner P F, Mayewski R J, Mushlin A I, Greenland P (1981) Annals of Internal Medicine, 94, 555-600]. When the results of a particular test in two populations, one population with a disease, the other population without the disease, are considered, a perfect separation between the two groups is rarely observed, and in fact the distribution of the test results will overlap. For every possible cut-off point or criterion value selected to discriminate between the two populations, there will be some cases with the disease correctly classified as positive (TP=True Positive fraction), but some cases with the disease will be classified negative (FN=False Negative fraction). On the other hand, some cases without the disease will be correctly classified as negative (TN=True Negative fraction), but some cases without the disease will be classified as positive (FP=False Positive fraction), as schematically presented in the Table below.

Schematic outcomes of a test
Disease

| Test | Present | n | Absent | n | Total |
|---|---|---|---|---|---|
| Positive | True Positive | a | False Positive | c | a + c |
| | (TP) | | (FP) | | |
| Negative | False Negative | b | True Negative | d | b + d |
| | (FN) | | (TN) | | |
| Total | | a + b | | c + d | |

| | | | |
|---|---|---|---|
| Sensitivity | a | Specificity | d |
| | a + b | | c + d |
| Positive | Sensitivity | Negative | 1 - Sensitivity |
| Likelihood | 1 - Specificity | Likelihood | Specificity |
| Ratio | | Ratio | |
| Positive | a | Negative | d |
| Predictive | a + c | Predictive | b + d |
| Value | | Value | |

The following statistical terms can be defined:
Sensitivity: probability that a test result be positive when the disease is present (true positive rate, expressed as a percentage)=a/(a+b)
Specificity: probability that a test result be negative when the disease is not present (true negative rate, expressed as a percentage)=d/(c+d)
Positive likelihood ratio: ratio between the probability of a positive test result given the presence of the disease and the probability of a positive test result given the absence of the disease, i.e.=True positive rate/False positive rate=Sensitivity/(1-Specificity)
Negative likelihood ratio: ratio between the probability of a negative test result given the presence of the disease and the probability of a negative test result given the absence of the disease, i.e. =False negative rate/True negative rate=(1-Sensitivity)/Specificity
Positive predictive value: probability that the disease is present when the test is positive (expressed as a percentage)=a/(a+c)
Negative predictive value: probability that the disease is not present when the test is negative (expressed as a percentage)=d/(b+d)

When a higher criterion value is selected, the false positive fraction will decrease with increased specificity but on the other hand the true positive fraction and sensitivity will decrease.

When a lower criterion value is selected, then the true positive fraction and sensitivity will increase. On the other hand the false positive fraction will also increase, and therefore the true negative fraction and specificity will decrease.

In a Receiver Operating Characteristic (ROC) curve the true positive rate (Sensitivity) is plotted in function of the false positive rate (100-Specificity) for different cut-off points. Each point on the ROC plot represents a sensitivity/specificity pair corresponding to a particular decision threshold. A test with perfect discrimination (no overlap in the two distributions) has a ROC plot that passes through the upper left corner (100% sensitivity, 100% specificity). Therefore the closer the ROC plot is to the upper left corner, the higher the overall accuracy of the test [Zweig & Campbell, 1993, ibid.].

Example 3 illustrates the general strategy that may be adopted for designing pair or triplets (or other subsets of antigens) of antigens that can distinguish between healthy (negative) and ill (positive) examined subjects. It is to be remembered that the diagnostic method and kit of the invention can be designed with differing specificity and sensitivity, according to requirements by an attending physician, that are determined on basis of the nature of the disease, the patient to be examined, epidemiological and statistical information, to name but few. By way of example, for diagnosing ovarian carcinoma, a very lethal type of cancer, higher sensitivity. i.e., a lower number of false negatives, is most important even if the number of false positives tends to be higher, and the attending physician require a most sensitive test. On the other hand, for example for breast cancer, which is less lethal, higher specificity, i.e., a lower number of false positives may be more important. As mentioned above, the threshold may be determined according to specific preferences and needs of specificity versus sensitivity. The analytic statistical methods employed by the invention, may answer needs of specific tests. It is thus a further embodiment of the invention to provide methods and means for selecting antigens for the diagnostic methods of the invention, which, together with suitable analytical means, provide for specifically designed accurate and reliable diagnostic tests. In some embodiment, specifically designed accurate and reliable diagnostic tests is provided in Example 8.

As used herein to describe the present invention, “tumor”, “cancer”, “malignant proliferative disorder” and “malignancy” all relate equivalently to a hyperplasia of a tissue or organ. If the tissue is part of the lymphatic or immune systems, malignant cells may include non-solid tumors of circulating cells. Malignancies of other tissues or organs may produce solid tumors. In general, non-solid and solid tumors are, for example, carcinoma, melanoma, leukemia, and lymphoma.

Cancer and tumors include, but are not limited to, myeloid leukemia such as chronic myelogenous leukemia, acute myelogenous leukemia with maturation, acute promyelocytic leukemia, acute non-lymphocytic leukemia with increased basophiles, acute monocytic leukemia, acute myelomonocytic leukemia with eosinophilia, malignant lymphoma, such as Burkitt's non-Hodgkin's, lymphocytic leukemia, such as acute lymphoblastic leukemia, chronic lymphocytic leukemia, myeloproliferative diseases, solid tumors such as benign meningioma, mixed tumors of salivary gland, tumors in lip and oral cavity, pharynx, larynx, paranasal sinuses, colonic adenomas, adenocarcinomas, such as small cell lung cancer, kidney, uterus, prostate, bladder, ovary, colon, sarcomas, liposarcoma, myxoid, synovial sarcoma, rhabdomyosarcoma (alveolar), extraskeletal myxoid chondrosarcoma, Ewing's tumor, other include testicular and ovarian dysgerminoma, retinoblastoma, Wilms' tumor, neuroblastoma, malignant melanoma, mesothelioma, breast, skin, prostate, and ovarian cancer, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

The methods described herein for detection of autoantibodies and diagnosis of cancer, can be suitable for any stage in cancer. These methods may prove most advantageous for example for the diagnosis of breast cancer, compared, to mammography, which utilizes dangerous levels of radiation and causes great discomfort to the patient, besides having a relatively high degree of false positive results (which translates into numerous patients being submitted to further biopsy, accompanied by unnecessary anxiety, which is redundant in such cases of false positive results). The diagnostic method described in the present invention is based on a simple blood test, and has potentially a much smaller incidence of false positive, as well as false negative results, as it has high sensitivity as well as high specificity. The approach of assessing the specificity and sensitivity of the method of the invention are described in Example 3. Current macro-level diagnostic tools—mammogram, Digital Rectal Examination (DRE) and ultrasound (for breast, prostate and ovarian cancer, respectively) have the ability to diagnose cancer only after a suspicious tumor mass has already developed to a size that is visually detectable, resulting in lower survival rates and reduced quality of life for the patient. For example, for breast cancer, in the United States alone, around 30 million mammography procedures are undertaken annually and more than one million surgical breast biopsies are performed on women with suspicious breast lesions.

As defined herein “sample” refers to any sample obtained from an organism. Examples of biological samples include body fluids and tissue specimens. The source of the sample may be derived from such physiological media as blood, serum, plasma, saliva, sputum, breast milk, pus, tissue scrapings, washings, urine, tissue, such as lymph nodes, or the like. Tissue specimens include biopsies of spleen, lymph nodes, and any lymphocyte-containing tissue. Tissue samples may include biopsies of the tumor itself. A preferred sample is a plasma sample.

The present invention also provides a kit for the diagnosis of cancer. Essentially, the kit provides reagents to detect the presence of suitable antigen-autoantibody complex in a sample from a subject, said antibody being specifically reactive to the suitable antigen, as herein defined, or an immunoreactive fragment thereof. The kit includes at least two suitable antigens, or more, which may or may not be bound to a solid support, a secondary antibody which is reactive (or binds) to the antigen-recognizing autoantibody and a reagent for detecting the reaction/binding of the secondary antibody with the antigen-recognizing antibody, and instructions for use.

The kit is essentially designed for the detection of at least two antigen-autoantibody complexes in a subject, but may of course comprise the necessary reagents for the detection of more than two complexes, for example a triplet of complexes. The subject may be a cancer patient, or a healthy individual.

In one embodiment, such a kit is an antibody capture assay kit, such as an ELISA kit, which comprises a solid support, antigen/s, secondary antibodies when appropriate, and any other necessary reagents such as detectable moieties, enzyme substrates and color reagents as described above. The antibody capture diagnostic kit is, alternatively, an immunoblot kit generally comprising the components and reagents described herein. The particular reagents and other components included in the diagnostic kits of the present invention can be selected from those available in the art in accord with the specific diagnostic method practiced in the kit. Such kits can be used to detect at least two antibodies in biological samples, such as tissue or body fluid, particularly plasma, obtained from a subject.

In another embodiment, the kit may further comprise a vacuum sealed container for collecting blood samples, and means for obtaining plasma or serum therefrom.

The instructions for use may include instructions for the laboratory technician as well as for establishing standardization curves, reference or relative ratios, as herein described, dilutions to be applied, etc.

The present invention is further relating to a computer implemented diagnostic method 100 for use in assigning a diagnosis to a diagnosed subject being assessed for the presence of cancer. Cancer can be either breast or ovarian cancer. In other embodiments, cancer is colon, lung or prostate cancer.

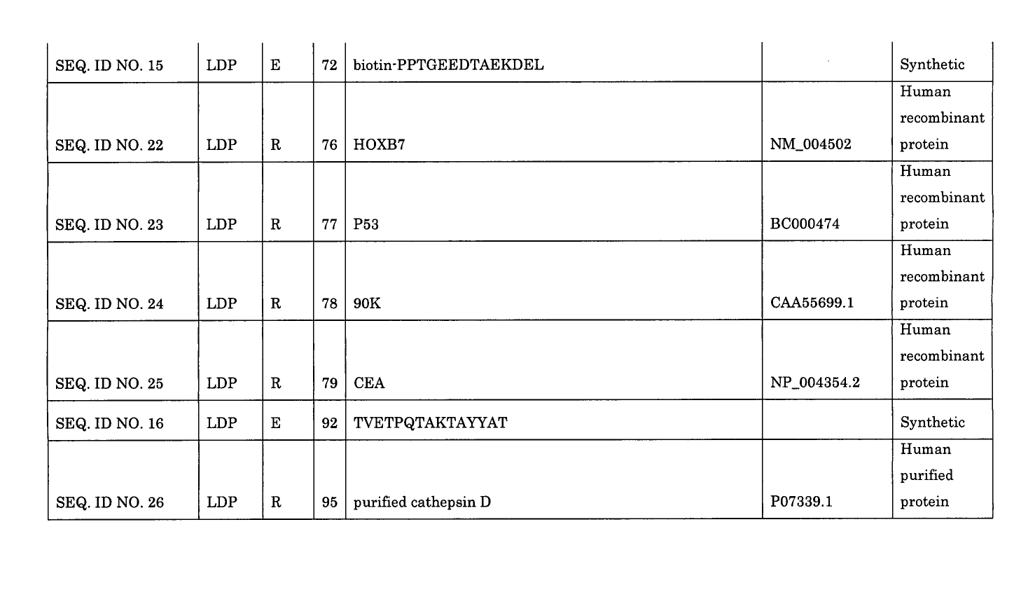

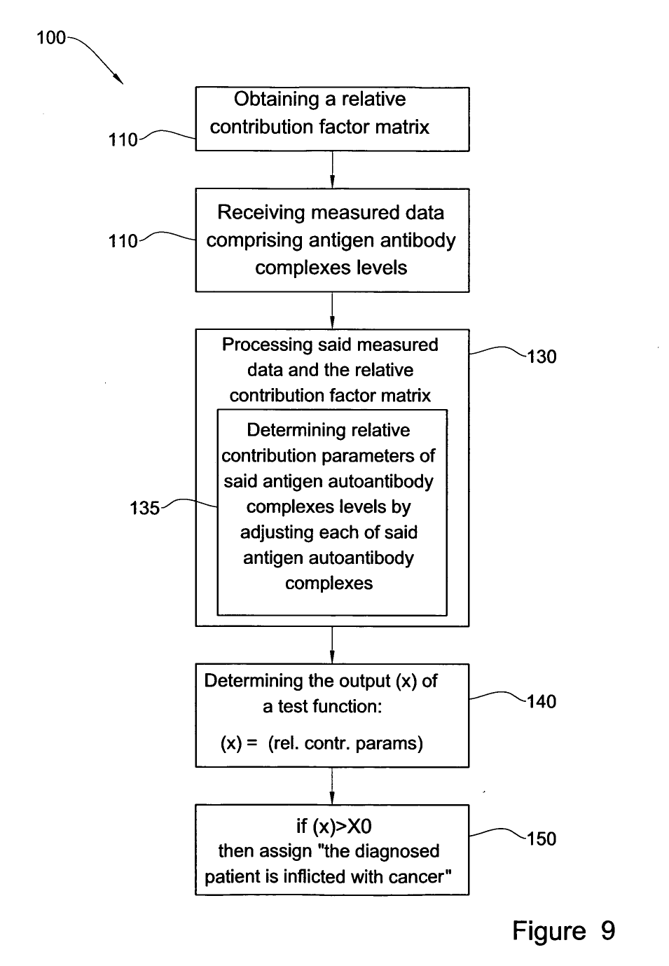

Reference is now made to FIG. 9 showing the computer implemented diagnostic method 100 in accordance with an embodiment of the invention. This method comprises receiving measured data comprising antigen-antibody complexes levels being obtained by contacting a body sample of said diagnosed subject with a predetermined set of antigens to form complexes with autoantibodies of said sample; wherein each of said antigens is characterized by said predetermined relative contribution factor to the presence of cancer. The body sample can be plasma or serum sample.

The diagnostic method 100 may also comprise the step of obtaining a relative contribution factor matrix; the relative contribution factor matrix comprises at least two predetermined relative contribution factors 110. Each pair of relative contribution factors defines a predicated signal strength relationship between two autoantibodies in the body sample. The two antibodies are being measured by a contacting them with antigens suitable to form antigen-antibody complexes therewith; the antigens are characterised by the relative contribution factors. The predicated signal strength relationship between two autoantibodies is used to identify that the diagnosed subject is afflicted with cancer.

The values are the relative contribution factor matrix are fixed or constant for providing the diagnostic results.

Processing the measured data and said relative contribution factor matrix 130 comprises determining relative contribution parameters of the antigen-autoantibody complexes levels. This is performed by adjusting each of said antigen-autoantibody complexes levels i.e. the measure data, in accordance with the predetermined relative contribution factor.

Adjusting can be performed by increasing of decreasing the measured data (e.g. (oᵢ)) in correlation to the relative contribution factors (e.g. (bᵢ), for antigen (i)). The increasing or decreasing can be proportional to the size of relative contribution factor. By way of non-limiting example, where the value of the relative contribution factor is relatively high the measured data is substantially increased and visa-versa. By way of another non-limiting example, each relative contribution parameter can be calculated by the following value adjustment:

PARAMᵢ=oᵢbᵢ,1≧i≧n; or

PARAMᵢ=ln(oᵢbᵢ),1≧i≧n

(wherein PARAMᵢ, relative contribution parameter (i)).

The computer implemented diagnostic method 100 comprise determining the output (x) of a test function (x)=f(relative contribution parameters) 140. The relative contribution parameters are the input of test function or discriminant function (x). In some embodiments, the discriminant or test function is (x)=Σᵢ₌₁ⁿ PARAMᵢ or (x)=b₀+b₁o₁+b₂o₂+b₃o₃+ . . . +bₙoₙ.

The diagnostic method 100 comprises comparing 150 said function output (x) with a threshold preestablished for healthy subjects, whereby if said (x) is higher than said threshold, said system variable is assigned with a status according to which the diagnosed patient is inflicted with cancer. In other words, a diagnosed subject will be assigned or classified as “having increased likelihood of being afflicted with cancer” if x>Z (predetermined cup-point of threshold), and “healthy” otherwise.

The method 100 optionally comprises outputting an indication that the diagnosed subject is assigned with the status according to which the diagnosed subject is afflicted with cancer.

A computer program product for assigning a diagnosis to a diagnosed subject being assessed for the presence of cancer is also provided. The program can be provided on a computer readable medium having a computer program code stored therein that, when executed by a processor, causes a method 100 to be performed.

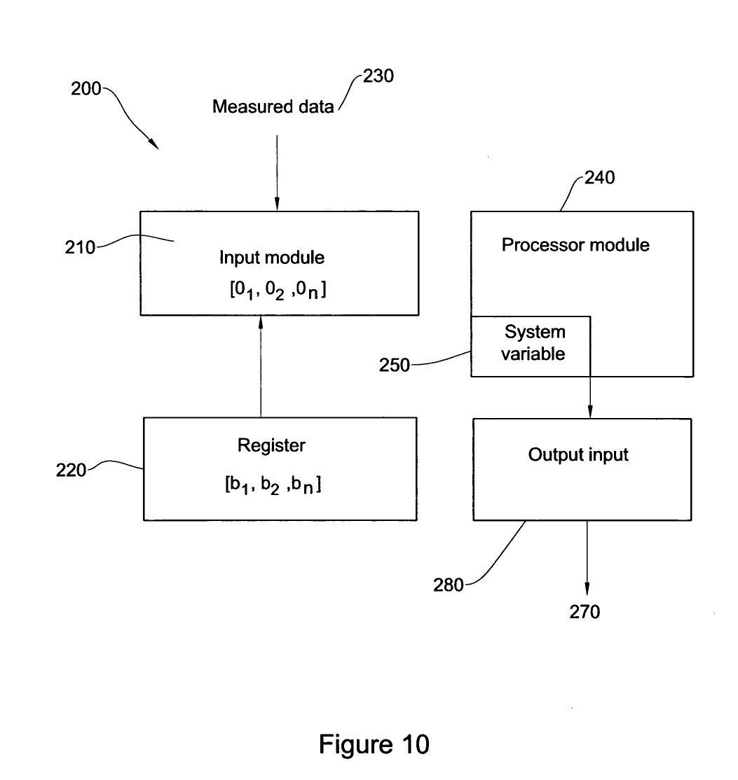

The present invention further provides a diagnostic monitoring system 200 which operates for diagnostics of cancer, as shown in FIG. 10. Cancer can be either breast or ovarian cancer. In other embodiments, cancer is colon, lung or prostate cancer.

The monitoring system assigns a diagnosis to a diagnosed subject being assessed for the presence of cancer. The system 200 comprises a register 220 for maintaining a relative contribution factor matrix. It is to be understood by the person skilled in the art that a register can be implemented by various way. By way of non-limiting example, a register may be a register of a processor based system or computer environment. A register can alternatively by obtained by allocation memory is a computer system such as RAM in which the relative contribution factor matrix would be stored or in other words registered. In addition, a register can alternatively employing by Read Only Memory (ROM) such as EPROM in which the relative contribution factor matrix would be permanently stored or in other words registered.

The values are the relative contribution factor matrix are fixed or constant for providing the diagnostic applications.

The relative contribution factor matrix comprises at least two predetermined relative contribution factors which are used to determine the relative contribution of tumor associated antigen to the occurrence of cancer in the diagnosed subject.

The relative contribution factor matrix comprises the proportional relationship of two or more antigen-antibody complexes levels characterizing the occurrence of cancer in said diagnosed subject.

The determination is performed by measuring the autoantibody levels in the body sample of the subject; the measurement being measurement of antigen-autoantibody complex levels. The body sample can be plasma or serum sample.

Input module 210 is used for receiving measured data comprising the antigen-autoantibody complexes levels being obtained by contacting a body sample of said diagnosed subject with a predetermined set of antigens to form complexes with autoantibodies of the body sample. These antigens or TAAs are characterized by the predetermined relative contribution factor to the presence of cancer in the diagnosed which are stored in the register.

A processor module 240 is used to process the measured data in conjunction with the relative contribution factor matrix. Processing is performed by determining relative contribution parameters for the measured antigen-autoantibody complexes levels. Each antigen-autoantibody complexes level is adjust (i.e. value correction) in accordance with relative contribution factor stored in the register.

Adjusting can be performed by increasing of decreasing the measured data (e.g. (oᵢ)) in correlation to the relative contribution factors (e.g. (bᵢ), for antigen (i)). The increasing or decreasing can be proportional to the size of relative contribution factor. By way of non-limiting example, where the value of the relative contribution factor is relatively high the measured data is substantially increased and visa-versa. By way of another non-limiting example, each relative contribution parameter can be calculated by the following value adjustment:

PARAMᵢ>oᵢbᵢ,1≧i≧n or PARAMᵢ=ln(oᵢbᵢ),1≧i≧n

(wherein PARAMᵢ, relative contribution parameter (i)).

Following the adjustment procedure output (x) of a test function (x)=f(relative contribution parameters) is determined. The computer implemented diagnostic method 100 comprise determining the output (x) of a test function (x)=f(relative contribution parameters) 140. The relative contribution parameters are the input of test function or discriminant function (x). In some embodiments, the discriminant function is (x)=Σᵢ₌₁ⁿ PARAMᵢ or (x)=b₀++b₂o₂+b₃o₃+ . . . +bₙoₙ.

If (x) is higher than a threshold pre-established for healthy subjects, system variable 250 is assigned with a value or status value according to which the diagnosed subject is afflicted with cancer.

The register 220 can store two relative contribution factors or more. The number of relative contribution factors is predetermined in accordance with the disease diagnosed or cancer type of the sensitivity or specificity demanded by the physician or otherwise.

The obtaining or receiving measurements for antigen-autoantibody complex levels can be performed such that a first aliquot of the body sample is obtained being diluted at a first dilution rate with a suitable buffer solution to provide measurable antigen-antibody complexes level. The first dilution rate can range at from 1:5 to 1:2000. Exemplary dilution rates are 1:5, 1:8, 1:10, 1:16, 1:24, etc. The obtaining or receiving measurements for antigen-autoantibody complex levels can be performed such that a second aliquot of the body sample, said second aliquot of said sample being diluted at a second dilution rate with a suitable buffer solution to provide measurable antigen-antibody complexes levels.

The second dilution rate can be different from the first dilution rate. Two measurable antigen-antibody complexes levels are thus obtained being from two different antigens at different dilution rates.

The first and second dilution rates define a relative dilution ratio of said two different antigens or a proportional size relationship of said two different antigens.

The techniques may find applicability in variety of computing or processing environments such a computer or a process based environments. The techniques may be implemented in hardware, software, or a combination of the two. The techniques may be implemented in programs executing on programmable machines such as stationary computers, and similar devices that each include a processor, a storage medium readable by the processor, at least one input device, and one or more output devices. Program code is applied to data entered using the input device to perform the functions described and to generate output information. The output information is applied to one or more output devices.

Each program may be implemented in a high level procedural or object oriented programming language to communicate with a processed based system. However, the programs can be implemented in assembly or machine language, if desired.

In another embodiment, the methods and systems can be utilized over a network computing system and/or environment. Number of computer systems could be coupled together via a network, such as a local area network (LAN), or a wide area network (WAN). The method 100 as a whole or a functional step thereof (110, 120, 130, 140, 150, or any combination thereof) could be thus implemented by a remote network computer or a combination of several. Any functional part of system 200 can be provided or connected via a computer network. By way of nonlimiting example, the register may be a remote register provide the factor matrix stored therein over the network. In addition, the processor module 240 can also be remotely providing the processor services over a network.

Each such program may be stored on a storage medium or device, e.g., compact disc read only memory (CD-ROM), hard disk, magnetic diskette, or similar medium or device, that is readable by a general or special purpose programmable machine for configuring and operating the machine when the storage medium or device is read by the computer to perform the procedures described in this document. The system may also be implemented as a machine-readable storage medium, configured with a program, where the storage medium so configured causes a machine to operate in a specific and predefined manner.

As used in the specification and the appended claims and in accordance with long-standing patent law practice, the singular forms “a” “an” and “the” generally mean “at least one”, “one or more”, and other plural references unless the context clearly dictates otherwise. Thus, for example “an antigen” may include one or more antigens.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

### General Methods in Molecular Biology

A number of methods of the molecular biology art are not detailed herein, as they are well known to the person of skill in the art. Such methods include PCR, expression of cDNAs, transfection of human cells, and the like. Textbooks describing such methods are, e.g., Sambrook et al. (1989) Molecular Cloning A Laboratory Manual, Cold Spring Harbor Laboratory, ISBN: 0879693096; F. M. Ausubel (1988) Current Protocols in Molecular Biology, ISBN: 047150338X, John Wiley & Sons, Inc. Furthermore, a number of immunological techniques are not in each instance described herein in detail, like for example Western Blot, as they are well known to the person of skill in the art. See, e.g., Harlow and Lane (1988) Antibodies: a laboratory manual, Cold Spring Harbour Laboratory.

### ELISA Protocol

Enzyme-linked Immunosorbent Assays (ELISAs) combine the specificity of antibodies with the sensitivity of simple enzyme assays, by using antibodies or antigens coupled to an easily-assayed enzyme. ELISAs can provide a useful measurement of antigen or antibody concentration. An ELISA is a five-step procedure: 1) coat the microtiter plate wells with antigen diluted in PBS, incubate at 4° C. and wash; 2) block all unbound sites to prevent false positive results in skim milk in PBS incubate 1 h and wash; 3) add antibody to the wells incubate 1 h and wash; 4) add anti-human IgG conjugated to an enzyme incubate 1 h and wash; 5) reaction of a substrate with the enzyme to produce a coloured product, thus indicating a positive reaction.

### Serial Dilutions

The sample may be serially diluted before being contacted with the antigen/s. For some tested subjects, serial dilutions of the samples may be necessary in order to overcome the detection limits of a given assay device. Where the at least two antigen-antibody complexes to be determined can be measured in the same dilution, the ratio between antigen-antibody complexes levels can be calculated from the direct measurement, as shown in FIG. 1A.

In cases where one of the complexes is considerably more concentrated than the other, it is not possible to use the same for both complexes. An example is shown in FIG. 1B, where the ratio between the complex levels cannot be determined because of device limitation (3>OD>0). In these cases, serial dilutions of at least two different dilutions or dilution rate (suitable for each of the complexes separately), will allow the calculation of the theoretical line [complex conc.]=f(dilution), or in case of an ELISA experiment [OD]=f(dilution). Using this line, the theoretical OD for any dilution can be calculated using extrapolation (FIG. 1C). After extrapolation, the ratio between both complexes can be determined for the same theoretical dilution.

### Peptide Antigens

Tables 1 and 2 present some specific antigens selected for the present study.

TABLE 1
Table 1-sequences of LDPE051, LDPE069, LDPE064 and LDPE070
Peptides were synthesized by Biomer Technology.

| SEQ ID NO. | Peptide # | Sequnce |
|---|---|---|
| 3 | LDPE-051 | Biotin-VATYAGQFNQDYLSGMAANMSGTFGGANMPNLY |
| 7 | LDPE-069 | Biotin-SQRIPARIHHFPTSI |
| 4 | LDPE-064 | biotin-EPPLSQETFSDLWKLLPENNVLSPL |
| 6 | LDPE-070 | Biotin-TVETPQTAKTAYYAT |

### Example 1

### Diagnosis of Potential Breast and Ovarian Cancer Patients

The present invention provides a simple method for the detection of a tumor in a subject via the immune system.

### Tested Subjects

Women with negative mammography results were considered healthy controls (CT); n=12 (LDPe051 and LDPe069), n=6 (LDPe064 and LDPe070).
   Women with cancerous breast biopsy or pathology were considered breast cancer patients (BC); n=10 (LDPe051 and LDPe069), n=7 (LDPe064 and LDPe070). Women with ovarian cancer as verified by pathology were considered ovary cancer patients (OC); n=8 (LDPe051 and LDPe069), n=7 (LDPe064 and LDPe070).

Blood samples of healthy controls and cancer patients (breast and ovary) were obtained with consent. Plasma was collected from the blood samples using heparin tubes after 10 min centrifugation, RT, 3000×g.

ELISA plates were coated with 4 different antigens (LDPe051—SEQ ID NO. 3; LDPe069—SEQ ID NO. 7; LDPe064—SEQ ID NO. 4; LDPe070—SEQ ID NO. 6) and blocked with 1% skim milk.

Plasma was diluted in 5% skim milk in PBS (1:30-1:4000) and loaded on 4 different ELISA wells, each containing a different antigen (LDPe051, LDPe064, LDPe069, LDPe070) and incubated at 37° C. for 1 h.

Plates were washed with 0.1% PBST and goat-anti human Ig HRP conjugate (diluted 1:5000 in blocking buffer) was added to each well and incubated at 37° C. for 1 h.

Plates were washed with 0.05% PBST and TMBE was added to each well.

Color development was stopped using 0.5M H₂SO₄ after 30 min and OD was measured at 450 nm.

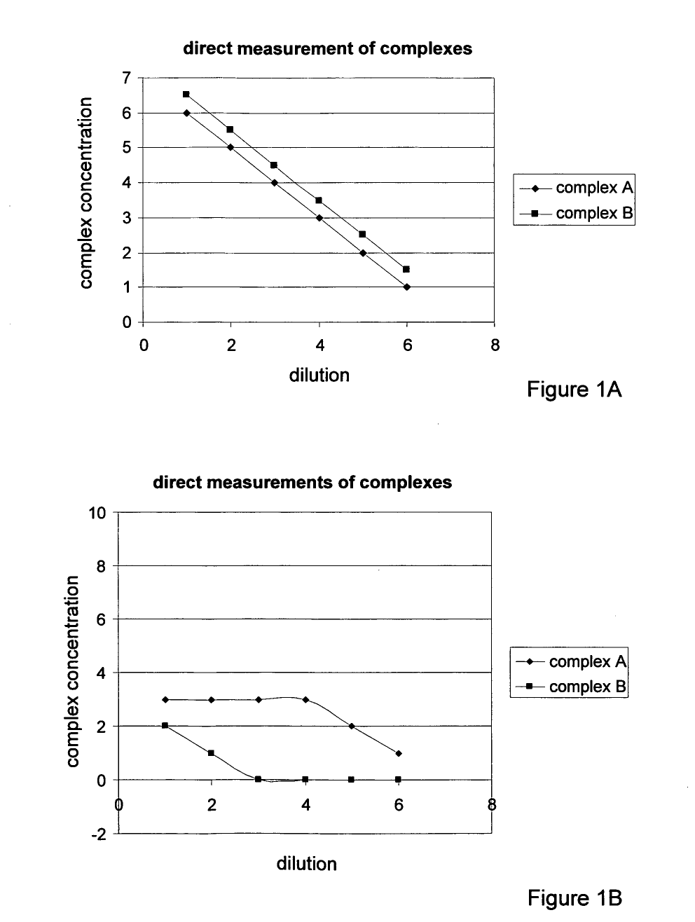

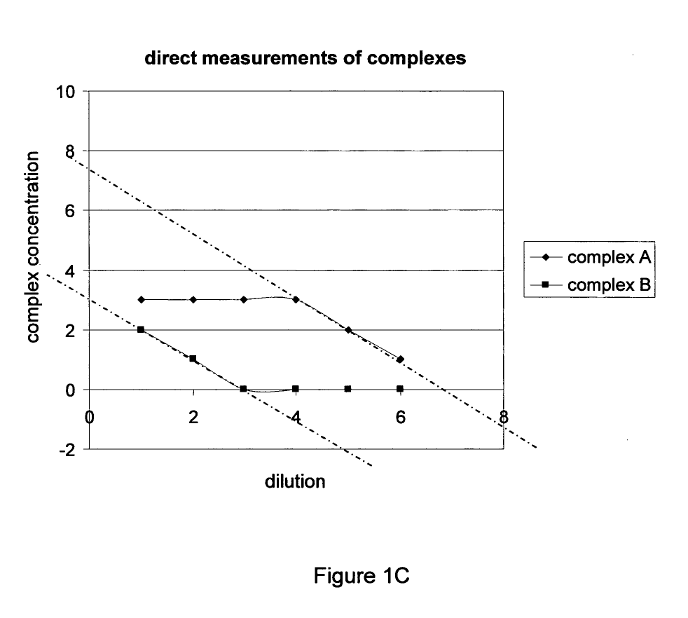

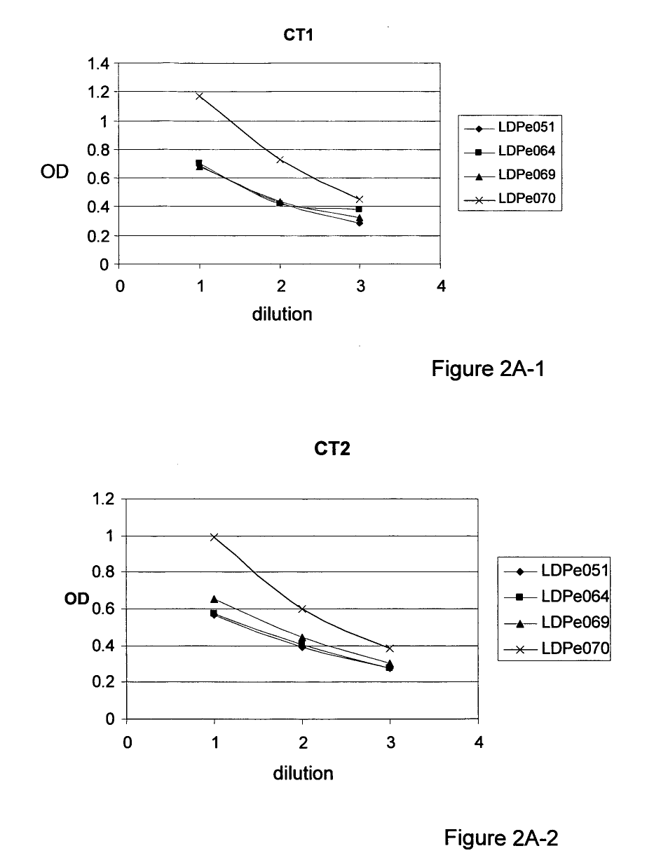

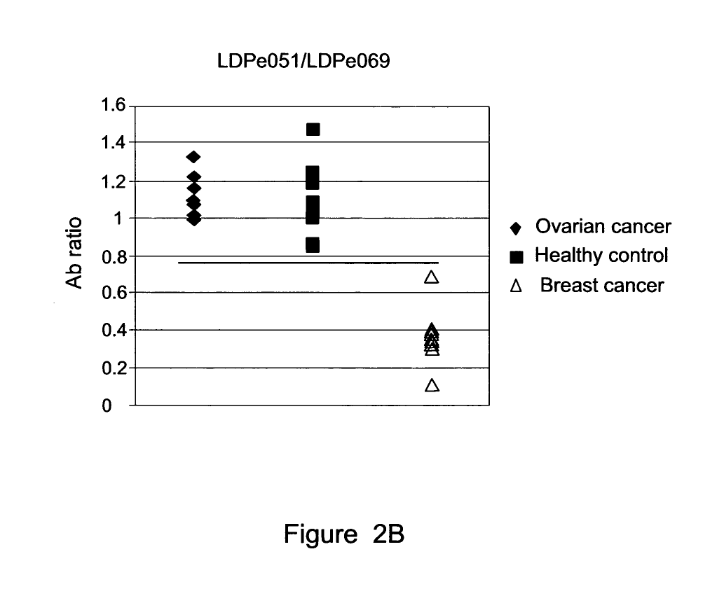

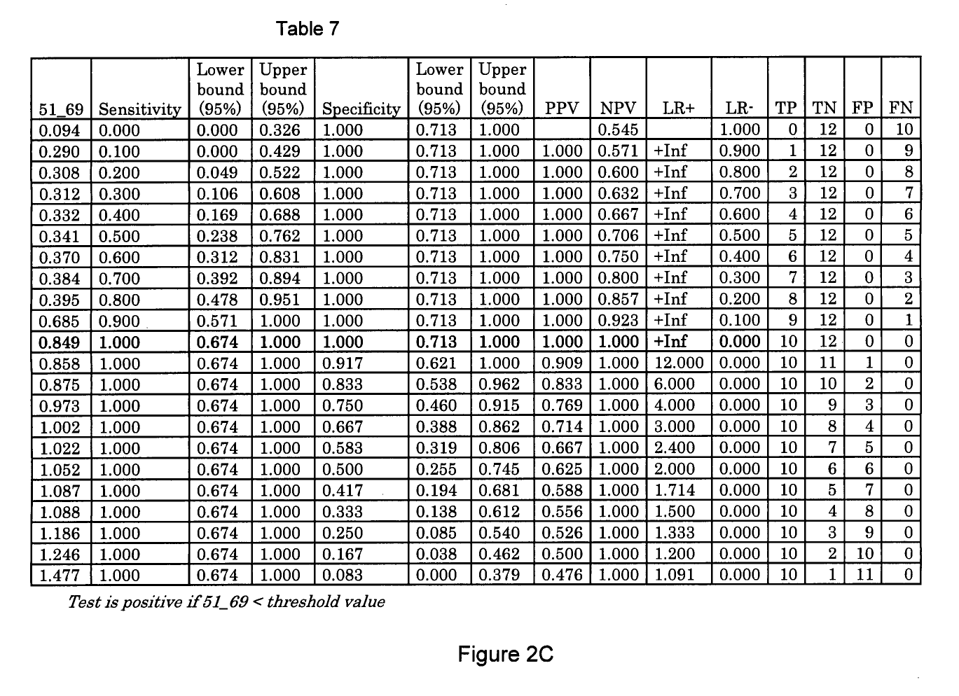

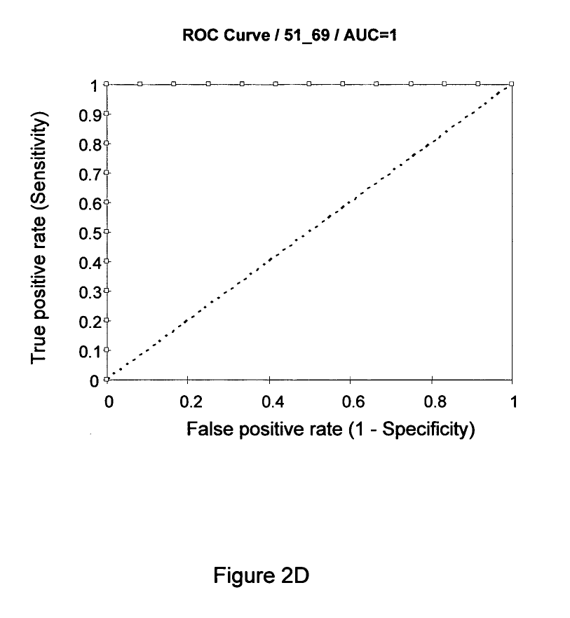

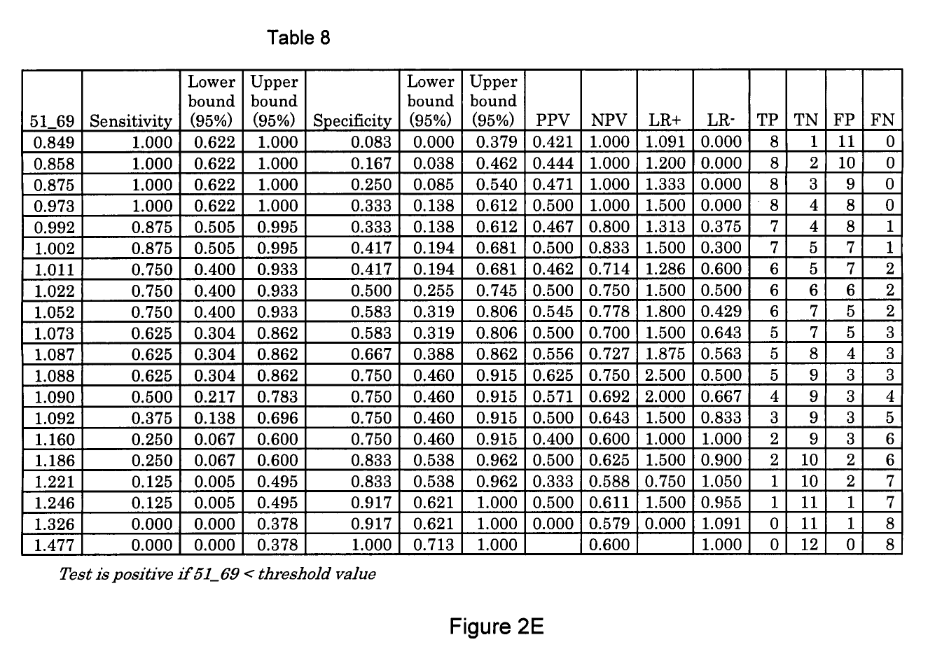

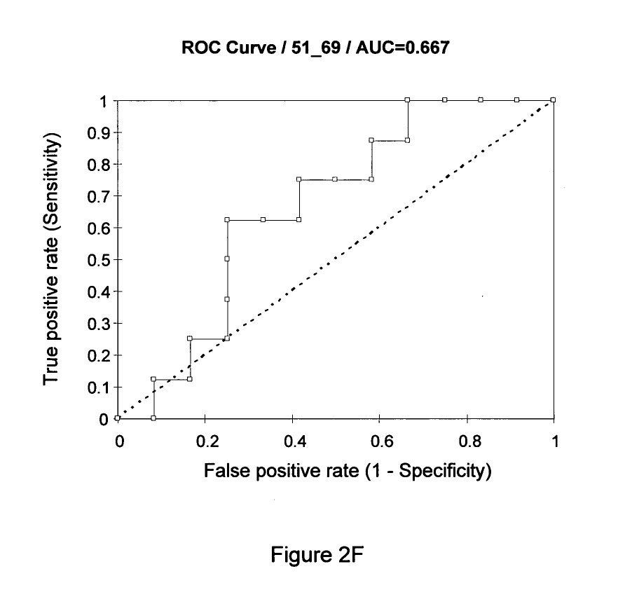

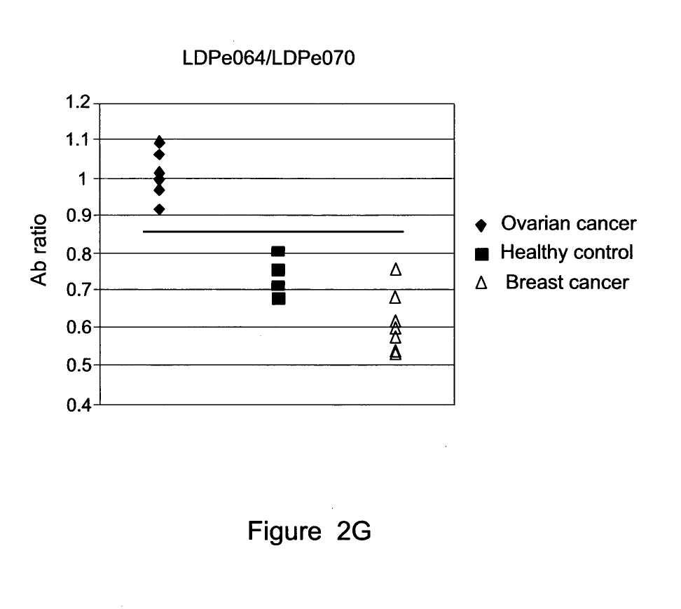

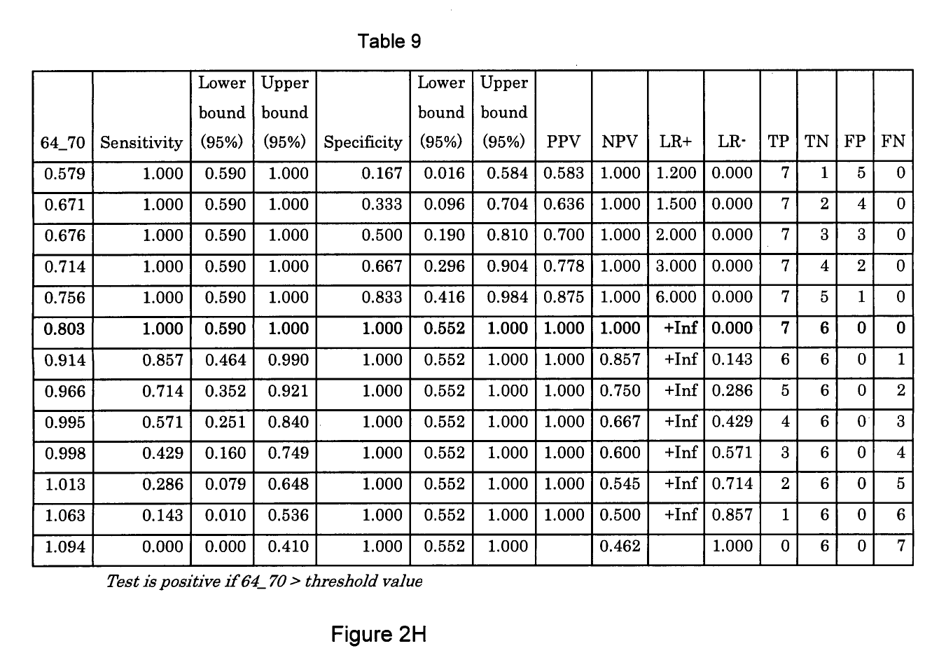

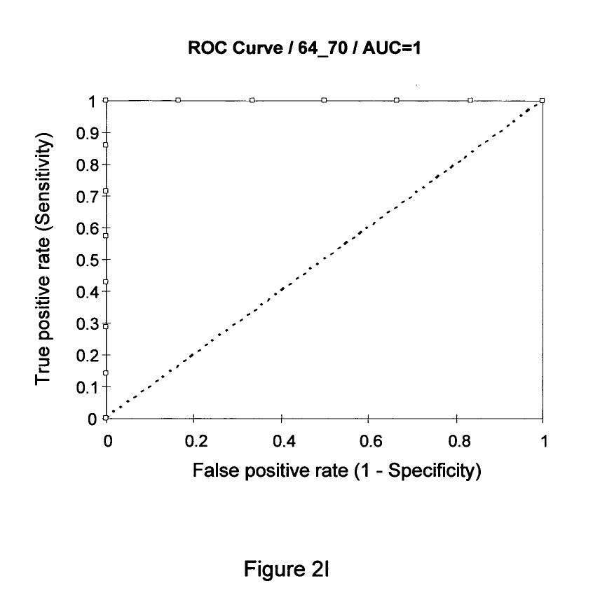

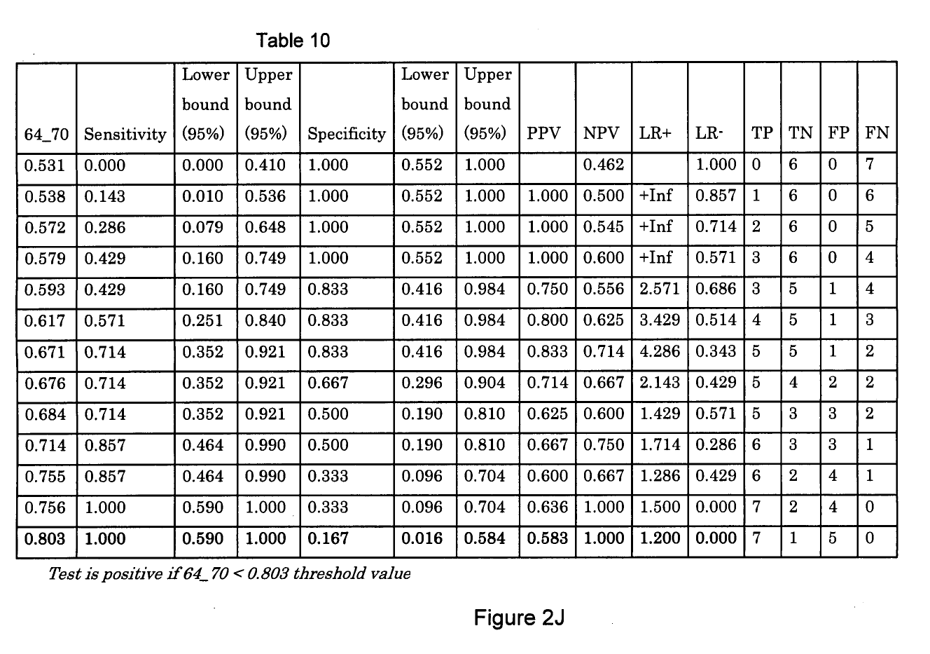

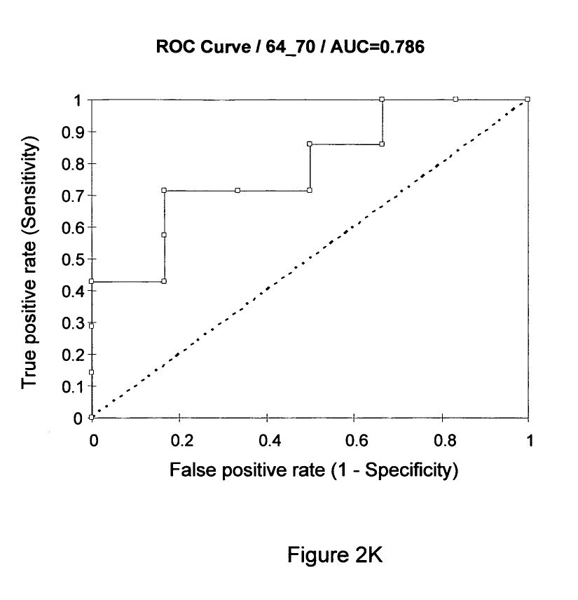

A sample of the results obtained for two healthy controls (CT1, CT2) are presented in Table 2 and FIG. 2A.

TABLE 2
Table 2 - OD results for 3 different dilutions for CT1 and CT2 for
4 different antigens (LDPE051, LDPE064, LDPE069, LDPE070)
and calculated ratio between two pairs (LDPEp51_LDPE069
and LDPE064_LDPE070)

| Dilu- | | | | | Ratio | Ratio |
|---|---|---|---|---|---|---|
| tion | LDPe051 | LDPe064 | LDPe069 | LDPe070 | 51_69 | 64_70 |

CT1

| | | | | | | |
|---|---|---|---|---|---|---|
| 1 | 0.687 | 0.702 | 0.684 | 1.169 | 1.00 | 0.60 |
| 2 | 0.425 | 0.421 | 0.437 | 0.727 | 0.97 | 0.58 |
| 3 | 0.285 | 0.38 | 0.323 | 0.447 | 0.88 | 0.85 |

CT2

| | | | | | | |
|---|---|---|---|---|---|---|
| 1 | 0.568 | 0.57 | 0.657 | 0.994 | 0.86 | 0.57 |
| 2 | 0.392 | 0.404 | 0.448 | 0.602 | 0.88 | 0.67 |
| 3 | 0.278 | 0.274 | 0.301 | 0.382 | 0.92 | 0.72 |

For dilution No. 2, the following results were obtained for the following samples (Table 3):

TABLE 3
OD results obtained for LDPe051 and LDPe069
and the calculated ratio (Dilution 2)

| Type | LDPe051 | LDPe069 | 51_69 |
|---|---|---|---|
| OC1 | 0.619 | 0.577 | 1.0728 |
| OC2 | 0.371 | 0.374 | 0.9920 |
| OC3 | 0.374 | 0.37 | 1.0108 |
| OC4 | 0.978 | 0.801 | 1.2210 |
| OC5 | 0.509 | 0.467 | 1.0899 |
| OC6 | 0.631 | 0.544 | 1.1599 |
| OC7 | 0.703 | 0.644 | 1.0916 |
| OC8 | 0.447 | 0.337 | 1.3264 |
| CT1 | 0.425 | 0.437 | 0.9725 |
| CT2 | 0.392 | 0.448 | 0.8750 |
| CT3 | 1.477 | 1.245 | 1.1863 |
| CT4 | 0.715 | 0.484 | 1.4773 |
| CT5 | 0.398 | 0.469 | 0.8486 |
| CT6 | 1.022 | 0.939 | 1.0884 |
| CT7 | 0.47 | 0.548 | 0.8577 |
| CT8 | 0.364 | 0.335 | 1.0866 |
| CT9 | 0.789 | 0.633 | 1.2464 |
| CT10 | 0.798 | 0.781 | 1.0218 |
| CT11 | 0.426 | 0.425 | 1.0024 |
| CT12 | 0.571 | 0.543 | 1.0516 |
| BC1 | 0.125 | 1.327 | 0.0942 |
| BC2 | 0.102 | 0.258 | 0.3953 |
| BC3 | 0.158 | 0.506 | 0.3123 |
| BC4 | 0.196 | 0.511 | 0.3836 |
| BC5 | 0.148 | 0.48 | 0.3083 |
| BC6 | 0.136 | 0.399 | 0.3409 |
| BC7 | 0.151 | 0.521 | 0.2898 |
| BC8 | 0.143 | 0.387 | 0.3695 |
| BC9 | 0.139 | 0.419 | 0.3317 |
| BC10 | 0.163 | 0.238 | 0.6849 |

OCx—ovarian cancer patient No. x
CTx—healthy control No. x
BCx—breast cancer patient No. x

The above results were plotted on a graph (FIG. 2B).

As shown in FIG. 2B, the ratio between LDPe051 and LDPe069 was different for breast cancer patients and healthy controls (a ratio<0.8 is indicative of a breast cancer patient while a ratio>0.8 represents healthy control). The ratio between the same two antigens in ovary cancer patient and healthy controls was identical (FIG. 2B).

ROC calculation for breast cancer—the results of the ratios between the two antigens were sorted in increasing order, and for each ratio point, the following values were calculated. Data is presented in Table 7, presented in FIG. 2C.

Threshold value determined to be 0.849—below which all tests were true negative (healthy) and above which all tests were true positive (breast cancer).

The ROC curve was plotted for each threshold point, as shown in FIG. 2D.

Similar calculation for the same antigens was performed for ovarian carcinoma patients (OC) and controls (CT). The results are presented in Table 8 (FIG. 2E).

As can be seen, when the pair of antigens LDPe051 and LDPe069 was used, good separation between healthy subjects and patients with ovarian cancer could not be obtained (where PPV and NPV are high). This can be further seen in FIG. 2F (ROC curve for LDPe051_LDPe069 for detection of ovarian cancer).

Using the pair of antigens LDPe064 and LDPe070, the following results were obtained (Table 4)

TABLE 4
OD results obtained for LDPe064 and LDPe070
and the calculated ratio (Dilution No. 2)

| type | LDPe064 | LDPe070 | 64_70 |
|---|---|---|---|
| OC1 | 0.612 | 0.613 | 0.998369 |
| OC2 | 0.432 | 0.434 | 0.995392 |
| OC3 | 0.511 | 0.467 | 1.094218 |
| OC4 | 0.542 | 0.535 | 1.013084 |
| OC5 | 0.63 | 0.652 | 0.966258 |
| OC6 | 0.699 | 0.765 | 0.913725 |
| OC7 | 0.387 | 0.364 | 1.063187 |
| CT1 | 0.421 | 0.727 | 0.579092 |
| CT2 | 0.404 | 0.602 | 0.671096 |
| CT3 | 0.47 | 0.695 | 0.676259 |
| CT4 | 1.009 | 1.257 | 0.802705 |
| CT5 | 0.705 | 0.933 | 0.755627 |
| CT6 | 0.802 | 1.123 | 0.714159 |
| BC1 | 0.147 | 0.215 | 0.683721 |
| BC2 | 0.243 | 0.322 | 0.754658 |
| BC3 | 0.24 | 0.405 | 0.592593 |
| BC4 | 0.211 | 0.369 | 0.571816 |
| BC5 | 0.163 | 0.307 | 0.530945 |
| BC6 | 0.162 | 0.301 | 0.538206 |
| BC7 | 0.226 | 0.366 | 0.617486 |

OCx—ovarian cancer patient No. x
CTx—healthy control No. x
BCx—breast cancer patient No. x

The above results were plotted on a graph (FIG. 2G).

As shown in FIG. 2G, the ratio between LDPe064 and LDPe070 was different for ovary cancer patients and healthy controls (a ratio>0.85 was indicative of an ovarian cancer patient while a ratio<0.85 represented healthy control). The ratio between the same two antigens in breast cancer patients and healthy controls was identical (FIG. 2G).

ROC calculation for ovarian cancer—the results of the ratios between the two antigens were sorted in increasing order, and for each ratio point, the values presented in Table 9 were calculated (FIG. 2H).

Threshold value determined to be 0.803—below which all tests were true negative (healthy) and above which all tests were true positive (breast cancer).

The ROC curve was plotted for each threshold point and is shown in FIG. 2I.

A similar calculation for the same antigens was performed for breast cancer (BC) patients and controls (CT). The results are presented in Table 10 (FIG. 2J).

As can be seen, using these antigens, good separation could not be obtained (where PPV and NPV are high).

The ROC curve obtained for the data of Table 10 is shown in FIG. 2K (LDPE064_LDPe070 for the separation between breast cancer and healthy controls).

In the above examples AUC=1, and the threshold point of the ratios was well defined as the point of 100% sensitivity and 100% specificity.

In other cases, where AUC<1, the threshold point of the ratios will be determined according to the clinical needs (high sensitivity or high specificity).

### Example 2

### Evaluation of the Diagnosis of Potential Breast Cancer Patients According to the Method of the Invention

Blood was collected from training set subjects with suspected breast cancer prior to biopsy and plasma was obtained, sample size determined by statistical considerations.

The pathological biopsy results were recorded as “Bpositive” (for cancerous sample) or “Bnegative” (for healthy sample).

Each sample was tested for the quantity of antigen-antibody complexes of antigens LDPe051 and LDPe069 according to the procedure in Example 1, and ratio between those complexes was determined.

If ratio>0.8, the sample was designated as “Rnegative”. If ratio is <0.8, the sample was designated as “Rpositive”.

Biopsy result for each sample (“Bpositive” or “Bnegative”) was compared to the ratio result (“Rpositive” and “Rnegative”).

Each sample was categorized as follows:

If sample was “Bpositive” and “Rpositive” then sample was true positive (TP)

If sample was “Bpositive” and “Rnegative” then sample was false negative (FN)

If sample was “Bnegative” and “Rpositive” then sample was false positive (FP)

If sample was “Bnegative” and “Rnegative” then sample was true negative (TN)

Specificity (TN rate) and sensitivity (TP rate) for the test was determined and plotted on a ROC (receiver operating characteristic) curve and AUC (area under curve) was calculated, as exemplified in Example 1.

AUC significantly higher than 0.5 demonstrates that there is a relationship between antibodies ratio and presence of breast cancer (BC). However, AUC<0.7 should be considered as insufficient for clinical use. A test with AUC between 0.7 and 0.85 will be considered as potentially useful in combination with other procedures, and the test with AUC>0.85 may be applied alone.

### Example 3

### General Strategy of Identifying Indicative/Diagnostic Pairs of Antigens

This example generally illustrates the strategy of identifying pairs of diagnostically indicative antigens, where the ratio between which complexes with their antibodies may be indicative of the presence or absence of cancer in a tested subject.

The strategy will be exemplified on basis of the peptides/proteins listed in Table 5

TABLE 5

| SEQ ID NO. | Peptide # | Sequence |
|---|---|---|
| 1 | LDP-E-007 | VFETLEEIT |
| | | (synthetic peptide, derived from Her2) |
| 2 | LDP-E-017 | YSQAVPAVTEGPIPEV |
| | | (synthetic peptide, derived from Chatepsin D) |
| 3 | LDP-E-051 | biotin-VATYAGQFNQDYLSGMAANMSGTFGGANMPNLY |
| 4 | LDP-E-064 | biotin-EPPLSQETFSDLWKLLPENNVLSPL |
| | | (synthetic peptide, derived from P53) |
| 5 | LDP-E-071 | NHEPSVTQVILDRPY |
| | | (synthetic peptide, outcome of a SEREX experiment) |
| 17 | LDP-R-062 | recombinant protein GRP78 GeneBank Accession No. |
| | | BC020235 |
| 18 | LDP-R-080 | recombinant protein Endostatin |
| 19 | LDP-R-084 | recombinant protein TPO |
| 20 | LDP-R-085 | recombinant protein Erbb2 |

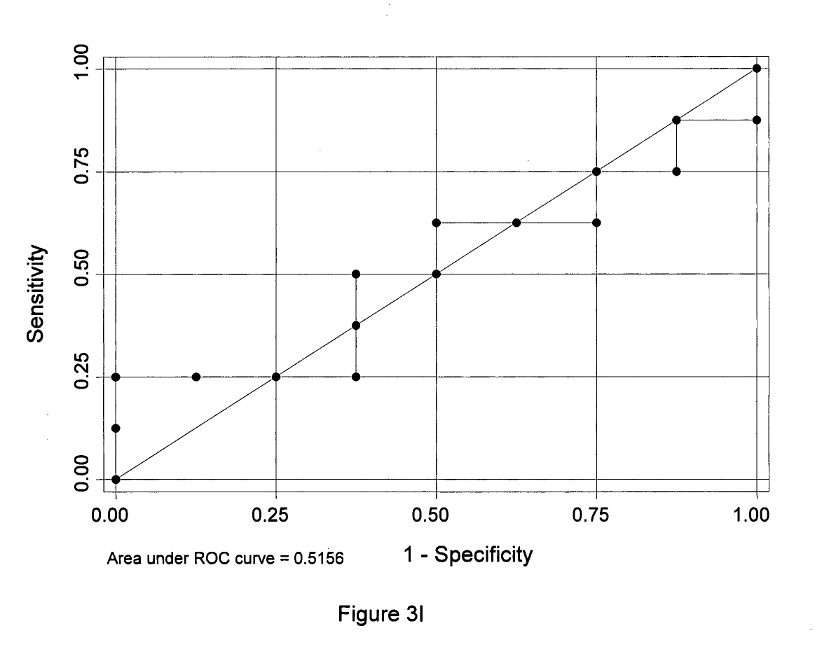

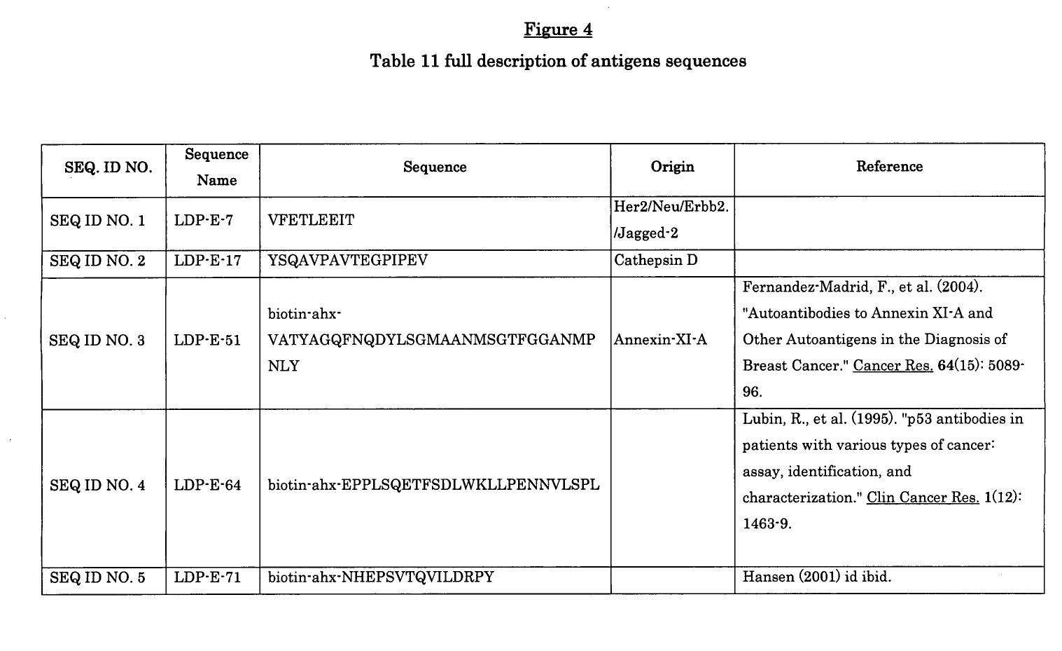

Full sequences are given in FIG. 4 (Table 11).

Blood samples from healthy individuals (“true negatives”) and individuals unequivocably diagnosed with breast cancer, e.g., by biopsy (“true positives”), were collected and plasma was obtained. An adequate number of positive and negative samples for statistical evaluations were collected. For each plasma sample, 9 different ELISA experiments were performed (as described in Example 1) using each of the antigens listed in Table 5. The actual levels of autoantibodies to each of the 9 antigens in each of the samples were determined from the ELISA results. All possible combinations of ratios between the autoantibody levels two complexes for each individual were calculated and the ratios from each individual for each antigen pair were compiled and the results were analyzed for AUC, according to known statistical methods (as in Example 1). All possible combinations were sorted by the calculated AUC. Antigen pairs showing higher AUC values are more diagnostically relevant whereas those pairs with AUC values approaching 50% are of much less value. As can be seen in Table 6, there are “good” combinations and “bad” combinations (according to AUC). The choice of the “good” pairs (such pairs the ratio between which can distinguish between positive and negative examinees to a predetermined extent e.g. at least 80% AUC) may entail a very large number of experiments. Such experimentation, following the methods described in detail herein (see, e.g., Example 1 above), however, is routine. The best pairs may be used as indicative/diagnostic antigen pairs.

Using all 9 antigens for ELISA of each sample, (9×8)/2 different combinations of ratios are possible, i.e. 36 pairs of different antigens can be used for ratio calculation, shown in Table 6 below.

TABLE 6
AUC analysis for 36 combinations of pairs of 9 antigens

| No. | x_x1 | x_x2 | Ncase | Ncont | AUC | chi2e |
|---|---|---|---|---|---|---|
| 1. | LDPr080 | LDPr085 | 8 | 8 | .96875 | 13.85967 |
| 2. | LDPr084 | LDPr085 | 8 | 8 | .953125 | 14.27417 |
| 3. | LDPe007 | LDPe017 | 12 | 11 | .9166667 | 14.8073 |
| 4. | LDPe007 | LDPr085 | 12 | 11 | .9015151 | 14.549 |
| 5. | LDPe017 | LDPe071 | 12 | 11 | .8939394 | 14.278 |
| 6. | LDPe071 | LDPr085 | 12 | 11 | .8560606 | 11.38461 |
| 7. | LDPe017 | LDPr085 | 12 | 11 | .8484849 | 7.713615 |
| 8. | LDPe051 | LDPr085 | 12 | 11 | .8484849 | 10.27533 |
| 9. | LDPr062 | LDPr085 | 12 | 11 | .8333333 | 8.91336 |
| 10. | LDPe017 | LDPe051 | 12 | 11 | .8333333 | 9.245241 |
| 11. | LDPe007 | LDPe064 | 12 | 11 | .8257576 | 9.150488 |
| 12. | LDPe017 | LDPe064 | 12 | 11 | .8181818 | 7.346355 |
| 13. | LDPe064 | LDPr085 | 12 | 11 | .8181818 | 8.926658 |
| 14. | LDPe017 | LDPr084 | 8 | 8 | .796875 | 4.674316 |
| 15. | LDPe017 | LDPr062 | 12 | 11 | .7954545 | 5.355608 |
| 16. | LDPe007 | LDPr080 | 8 | 8 | .78125 | 5.962902 |
| 17. | LDPe017 | LDPr080 | 8 | 8 | .78125 | 4.593035 |
| 18. | LDPe007 | LDPe051 | 12 | 11 | .7727273 | 6.398762 |
| 19. | LDPe007 | LDPe071 | 12 | 11 | .7651515 | 6.728445 |
| 20. | LDPe007 | LDPr062 | 12 | 11 | .7651515 | 6.088541 |
| 21. | LDPe064 | LDPe071 | 12 | 11 | .7348485 | 5.538493 |
| 22. | LDPe007 | LDPr084 | 8 | 8 | .734375 | 4.826306 |
| 23. | LDPr062 | LDPr084 | 8 | 8 | .671875 | .7952933 |
| 24. | LDPr080 | LDPr084 | 8 | 8 | .640625 | .4871385 |
| 25. | LDPr062 | LDPr080 | 8 | 8 | .640625 | 1.077615 |
| 26. | LDPe064 | LDPr080 | 8 | 8 | .625 | .5623482 |
| 27. | LDPe051 | LDPr080 | 8 | 8 | .625 | .4821599 |
| 28. | LDPe051 | LDPr084 | 8 | 8 | .59375 | .1031399 |
| 29. | LDPe071 | LDPr080 | 8 | 8 | .59375 | .6531609 |
| 30. | LDPe051 | LDPe064 | 12 | 11 | .5757576 | 1.803952 |
| 31. | LDPe064 | LDPr084 | 8 | 8 | .546875 | .1403739 |
| 32. | LDPe051 | LDPe071 | 12 | 11 | .5454546 | .3919335 |
| 33. | LDPe064 | LDPr062 | 12 | 11 | .5454546 | .7157161 |
| 34. | LDPe071 | LDPr062 | 12 | 11 | .530303 | .8007174 |
| 35. | LDPe051 | LDPr062 | 12 | 11 | .5227273 | .7236857 |
| 36. | LDPe071 | LDPr084 | 8 | 8 | .515625 | .2631851 |

x_x1—the first antigen used in the ELISA
x_x2—the second antigen used in the ELISA
Ncase—number of patients
Ncont—number of controls
AUC—area under curve calculated from the ROC curve for this antigen-antibody complex pair
Chi2e—statistical parameter (χ²)

It is to be understood that an object of the invention is to provide a diagnostic method that enables distinguishing between “ill” (patients with cancer) and “healthy” subjects. In a particular embodiment (as exemplified in Examples 1 and 2) the method is based on measurements of optical density (OD) of complexes formed with a couple or a triplet of antigens. Logistic regression may be used for finding the potentially efficient couples and triplets of antigens. The quality of a test is evaluated using Area Under the Receiver Operating Characteristic (ROC) Curve (AUC). The AUC [see for example Bamber D. J. Math. Psychol. (1975) 12:387-415; Hanley, J. A. and McNeil B. J., Radiology (1982) 143(1)29-36; D'Agostino R. B. et al., Proc. Biometrical Section. Alexandria, Va., USA American Statistical Association, Biometric section: Alexandria Va. (1997) 253-258] is still the most usable measure for evaluating model performance notwithstanding some new suggestions [Cook, N. R. Circulation (2007) 115:928-935; Pepe M. S., et al., UW Biostatistics Working paper Series, #289, (2006). Available at http://www.bepress.com/uwbiostat/paper289. accessed 9 Mar. 2007; Pencina M. J., et al., Medicine (2008) 27:157-172]. The AUC presents the sensitivity of a test as a function of its “one minus specificity”. The AUC gives the probability that out of two randomly and independently chosen samples (one “ill” and one “healthy”) the “ill” patient will have higher test value.

Based on such analysis, efficient characteristic pairs or triplets of antigens can be chosen, from a vast plethora of candidates to be used as the antigens in the proposed test.

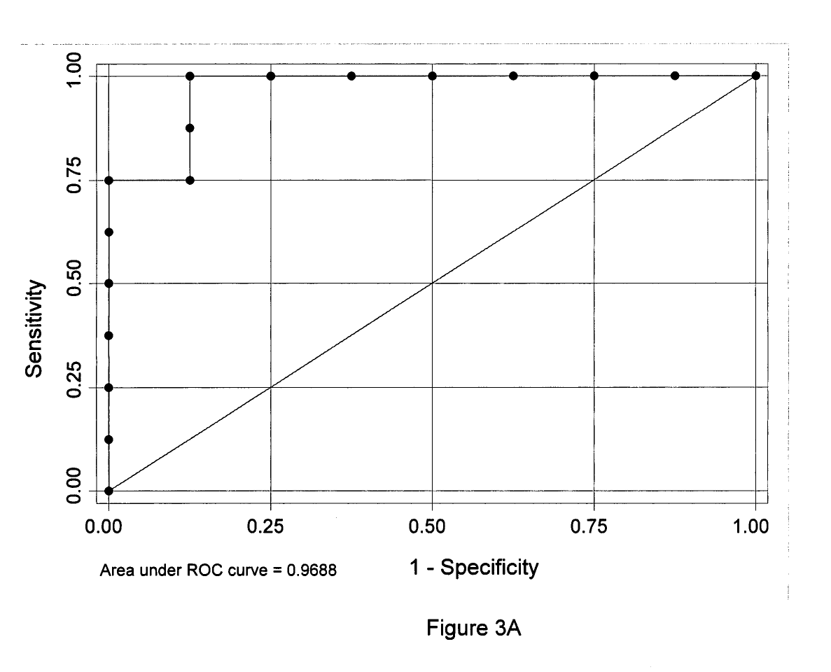

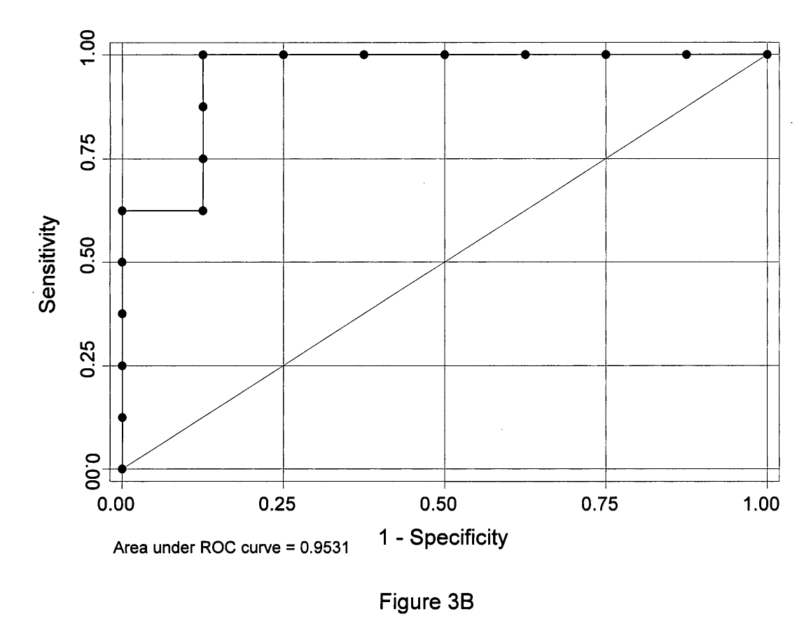

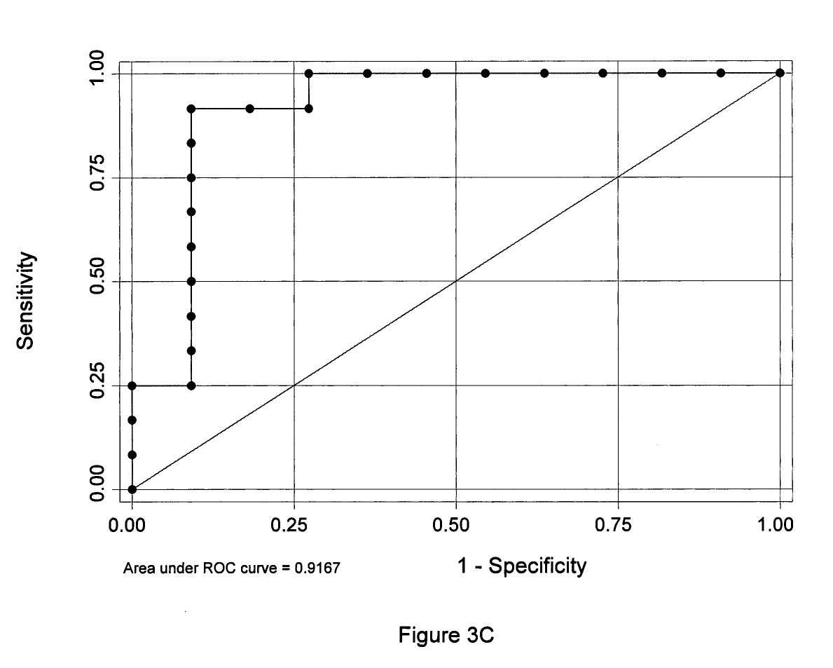

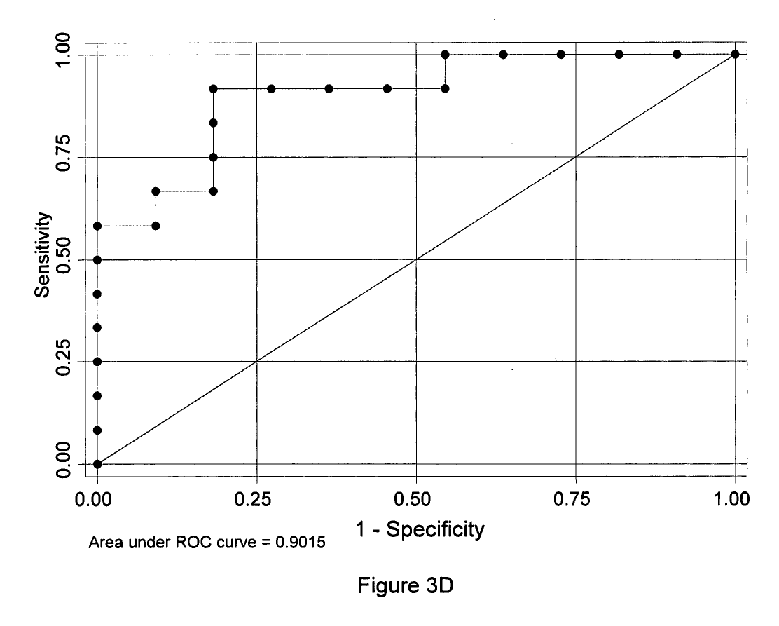

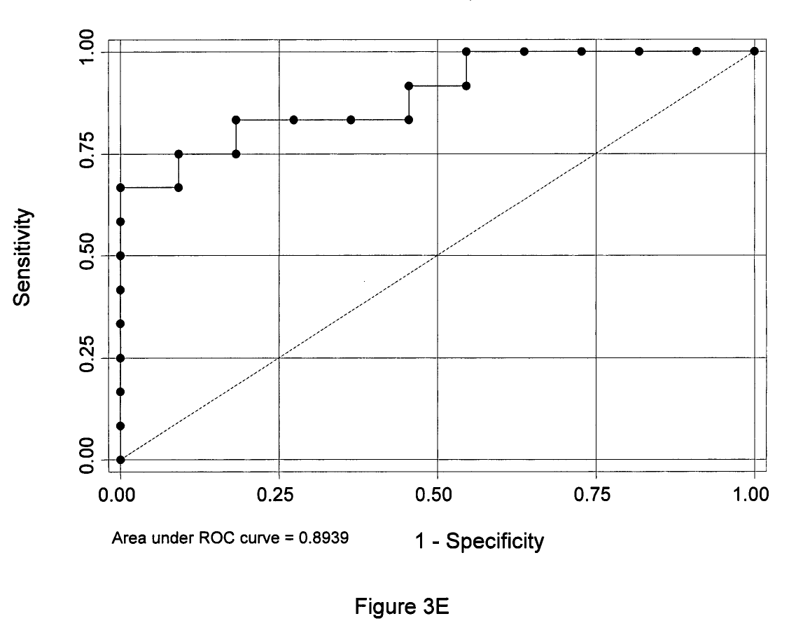

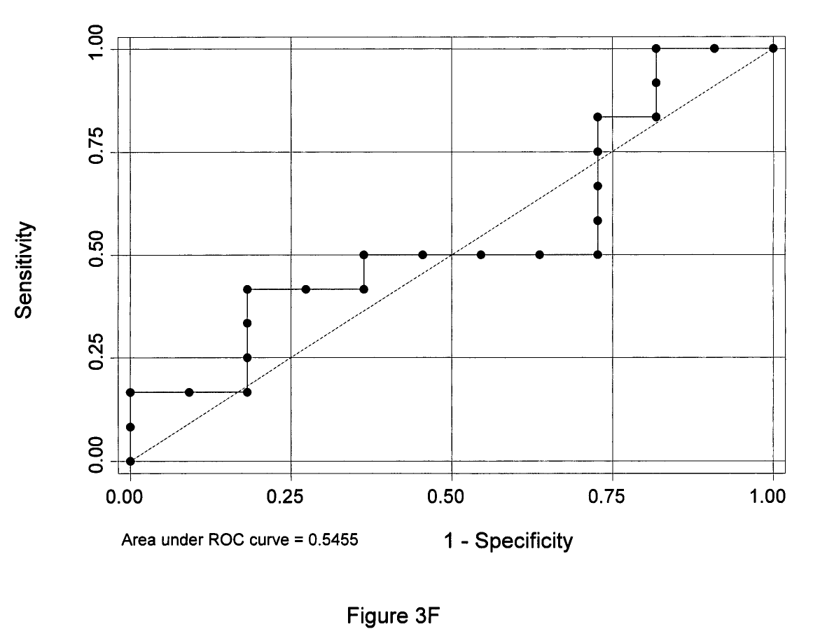

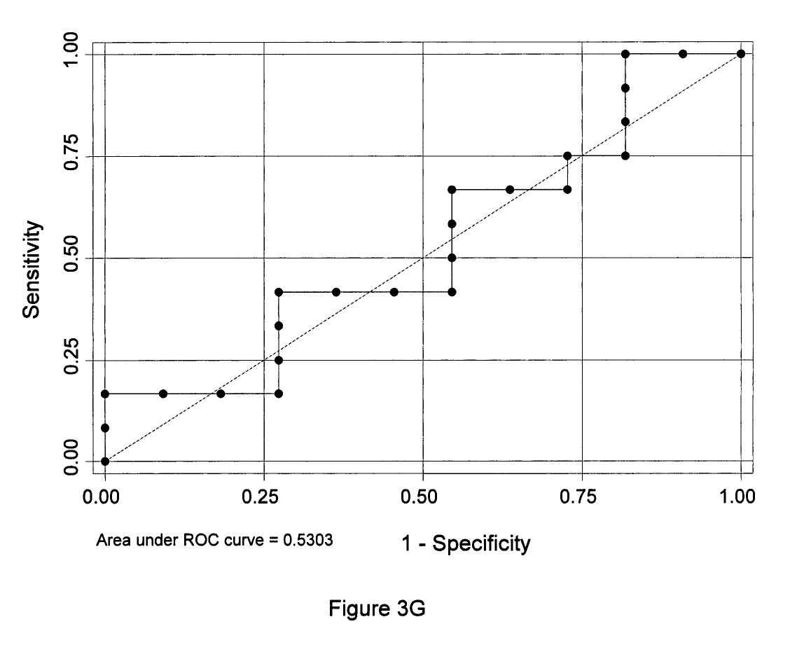

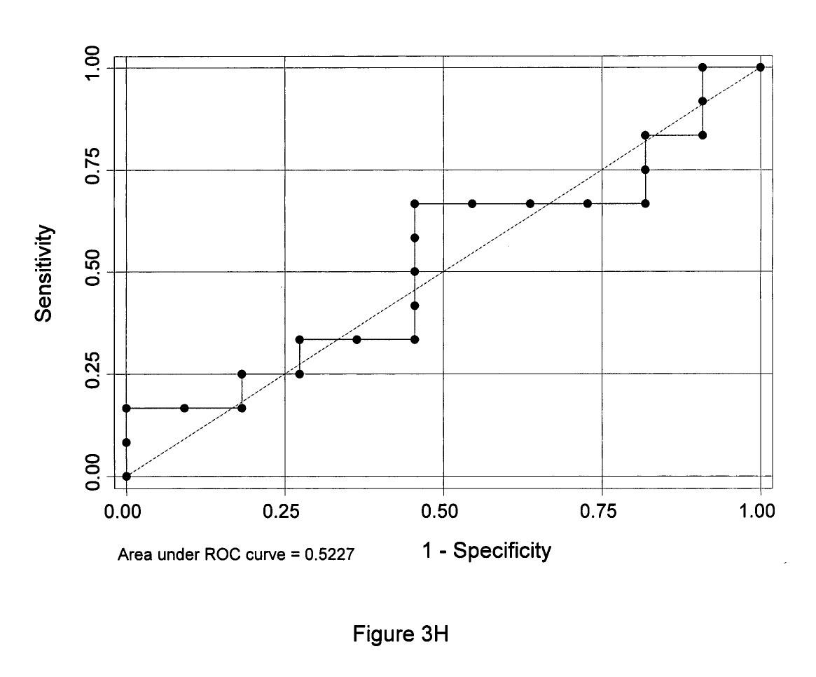

FIGS. 3A to 3I illustrate the method of selecting suitable antigens, i.e. such antigens that the quantitative ratio between complexes they form with antibodies present in the sample can efficiently distinguish between healthy subjects and those with cancer, by means of AUC analysis.

FIGS. 3A, 3B, 3C, 3D and 3E show the five best pairs from the above possible combinations (Table 6), yielding high AUC that can separate between populations (healthy and ill), and FIGS. 3F, 3G, 3H, and 3I show pairs yielding for low AUC (0.5) that cannot distinguish between the populations.

### Example 4

### Defining a Dilution Rate and Relative Dilution Ratios for Each of the Antibody-Antigens Complexes in the Diagnostic Set

Provided herein a diagnostic method and application that on one hand analyzes a body sample for measurements of the specific antibody-antigen complexes for plurality of antigens, each having different physiological or otherwise contribution to the occurrence of cancer and on the other hand, accounts for the diverse nature of antibody expression profiles across a population. As previously stated, a subject's production of autoantibodies may be characterized as “strong” having relatively substantial levels of a certain autoantibody in comparison to another subject's “weak” autoantibody production.

As demonstrated below, the diagnostic approach is flexible. Instead of performing a diagnostic assay on a body sample being diluted at a predefined single dilution rate (or series or rates) applicable for all antibody-antigen complexes which may be formed, a diagnostic assay was performed at plurality of dilutions rates. Collection and consolidation of the information gathered therefrom is shown. As illustrated below, a single range of dilutions for all antibody-antigen complexes for simultaneous use was not always available. Limiting the assay to a single dilution rate, posed a technical constraint for detecting device. Thus, a single dilution range or rates was not be applicable, because of assay device limitations. Inaccuracies occurred when one of the antibody-antigen complexes formed in the sample, as showed below, yielded a very high OD, and could not be measured with the specific detecting device used.

Thus, in the following example, different dilution rates were used to produce antigen-autoantibody complex levels which were measurable. The present example now illustrates that in order to perform an assay having plurality of detectable antigen-autoantibody complexes, different dilution rates can be used.

Two body samples were tested using 4 different antigens denoted as Ag1 (LDPe002)—SEQ ID No. 9, Ag2 (LDPe012)—SEQ ID No. 11, Ag3 (LDPr041)—SEQ ID No. 21, Ag4 (LDPr077)—SEQ ID No. 23, using two different modes of testing.

In the first mode, samples were diluted to the same dilutions (identical serial dilution) for all the antigens (starting with “Dilution 1” which denotes a dilution rate of 1:8). The results are summarized in Table 12. Table 12 provides the OD readings for test1139 and test1200 with respect to each of the antigens Ag1, Ag2, Ag3 and Ag4, in varying dilutions ranging from 1:8 to 1:256. Serial dilutions were the same for all antigens. Bold values are suitable for smoothing.

In the second mode, body samples were divided to several aliquots being serially diluted differently for each antigen. OD measurements of Ag1 started at a dilution rate of 1:8; for Ag2 the starting dilution rate was 1:32, for Ag3—1:64, and for Ag4—1:512. Results are summarized in Table 13. Table 13 provides OD readings for test1139 and test1200, for antigens Ag1, Ag2, Ag3 and Ag4, with different first dilutions Ag1—1:8, Ag2—1:32, Ag3—1:64, Ag4—1:512. Bold values represents suitable data for smoothing procedure.

OD readings were smoothed. The tests performed in the first mode, demonstrate that smoothing was not an option for some of the antigens at the starting dilution rate (1:8) as can be seen for test 1139, the raw data was as described in Table 12.

Clearly, a plurality of OD measurements in table 12 indicate an excessively high signal indicating saturation of the signal.

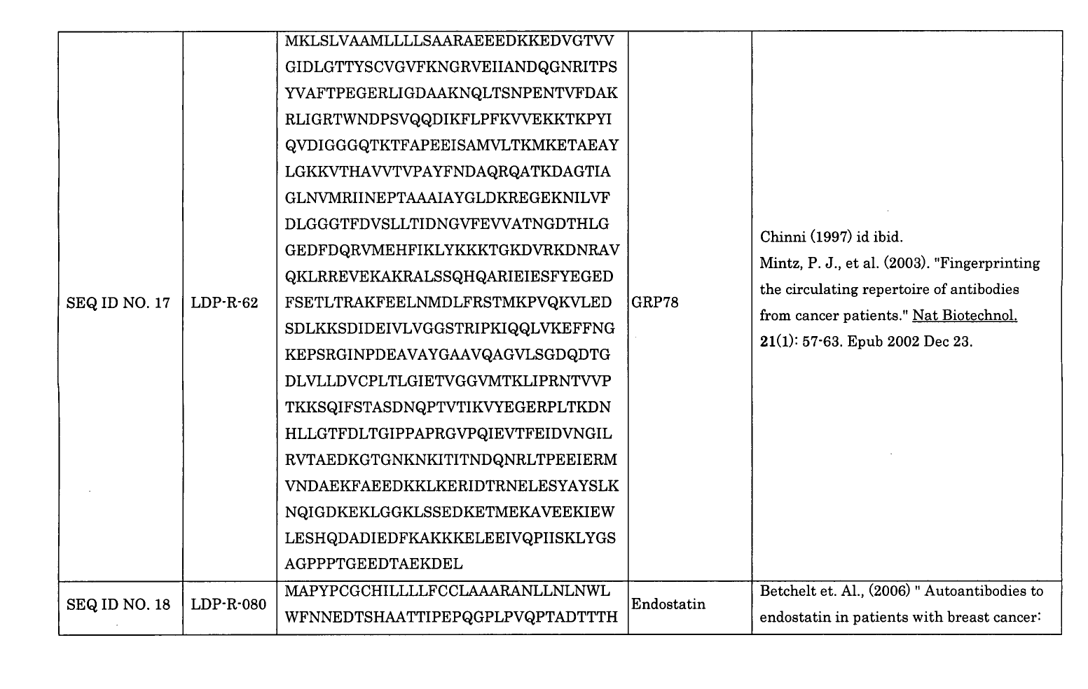

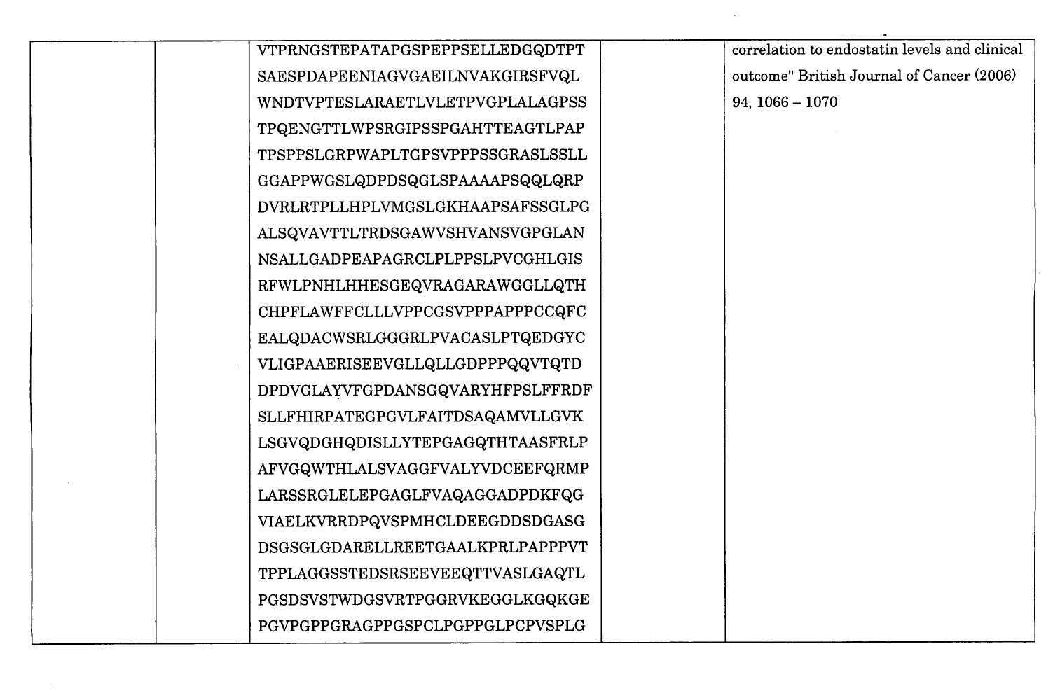

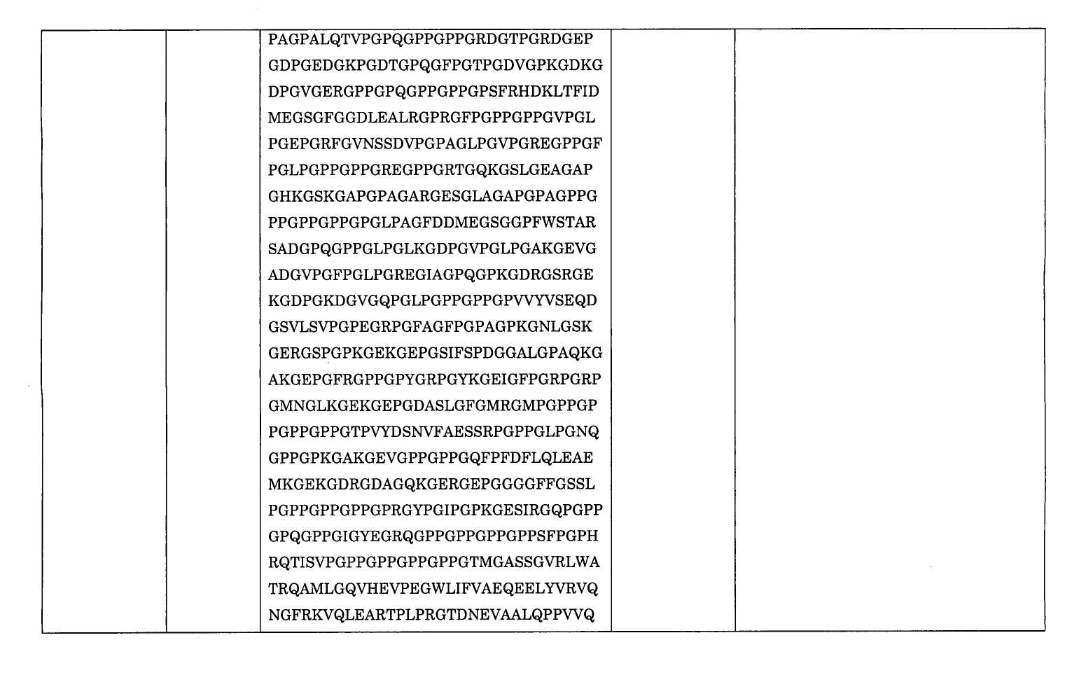

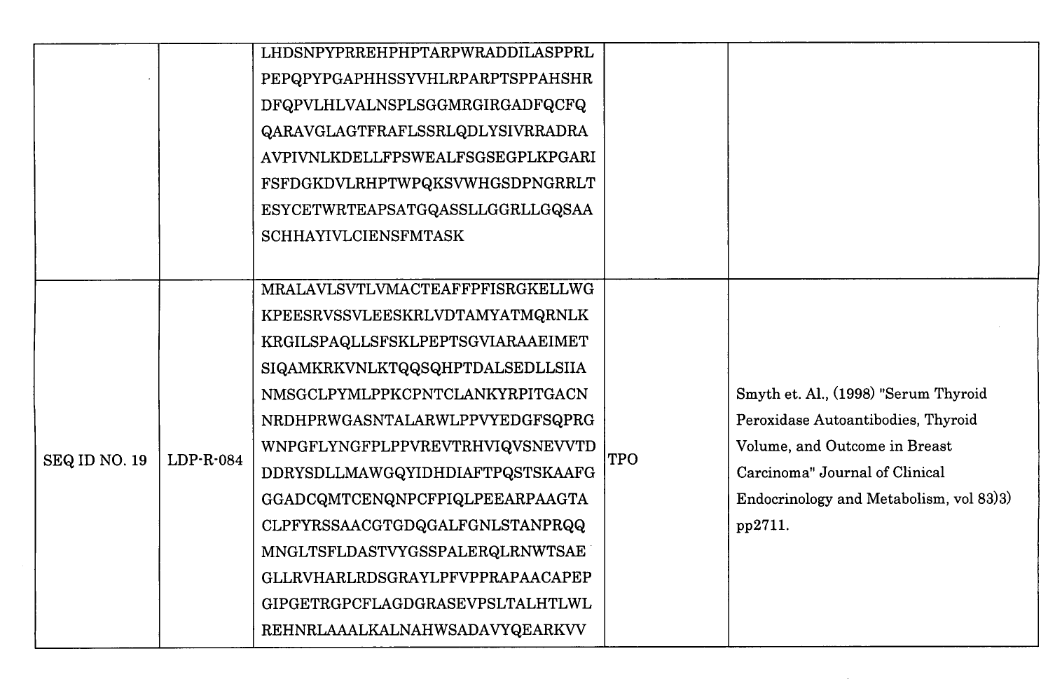

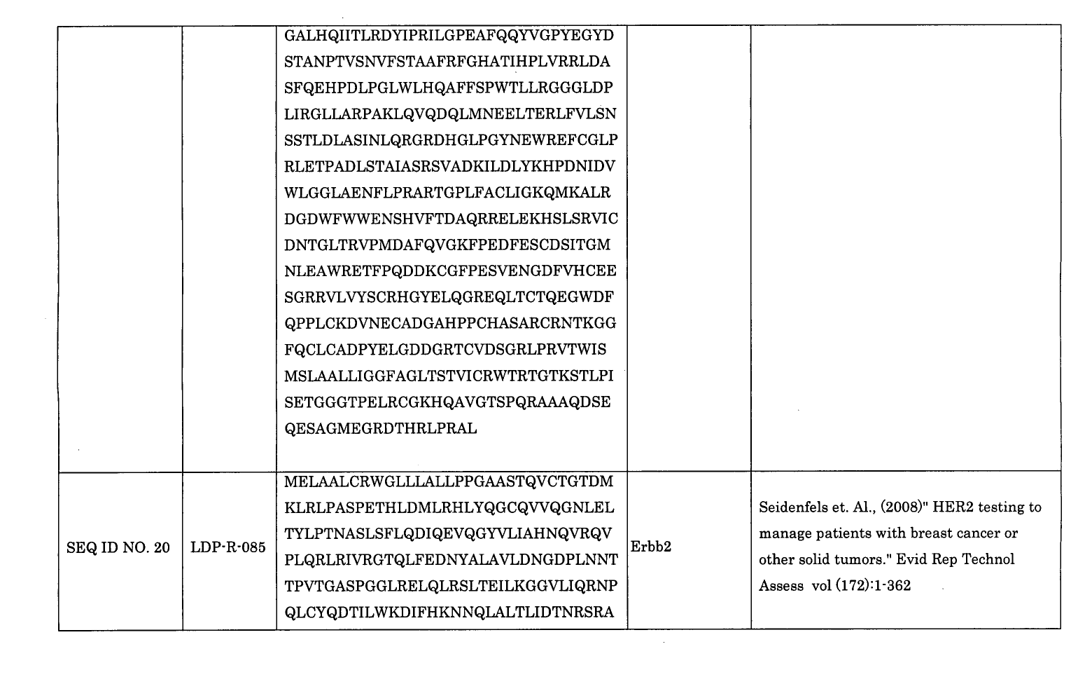

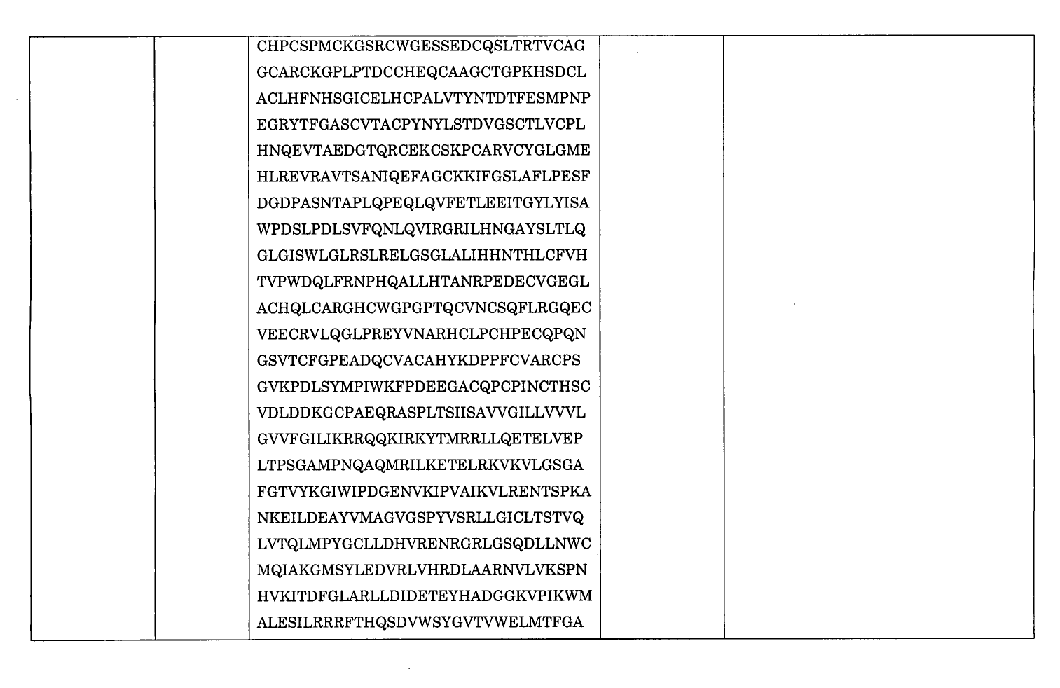

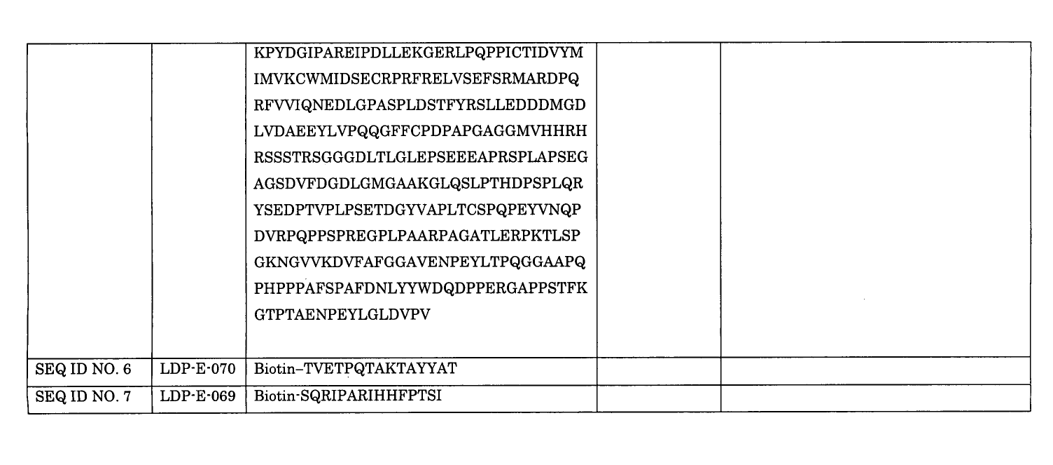

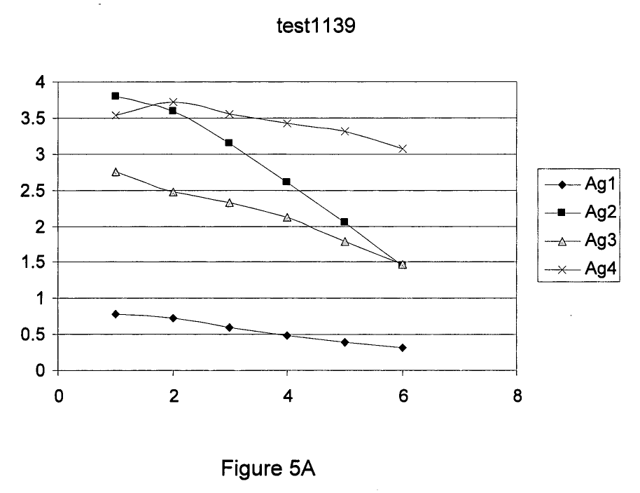

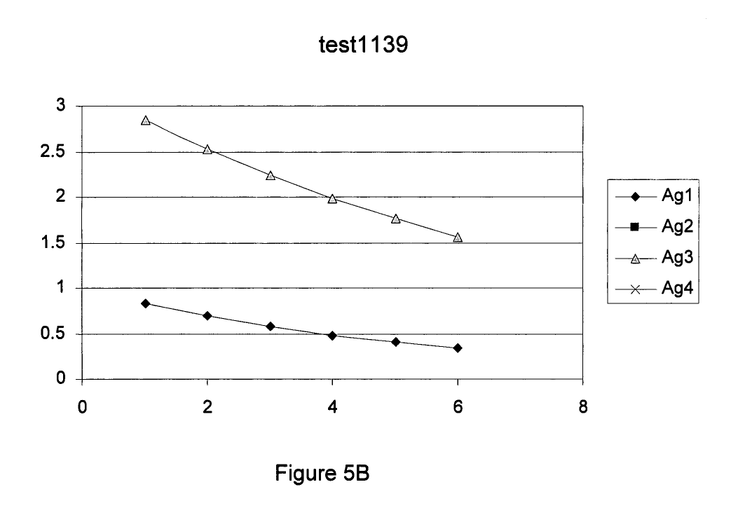

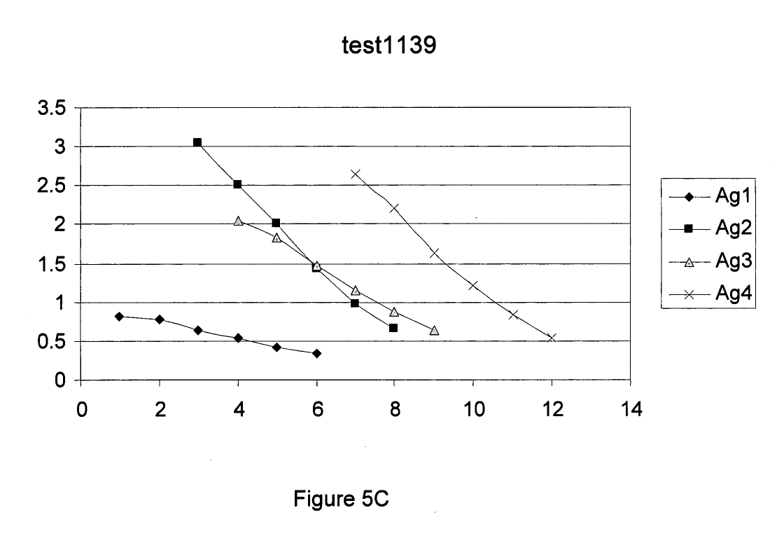

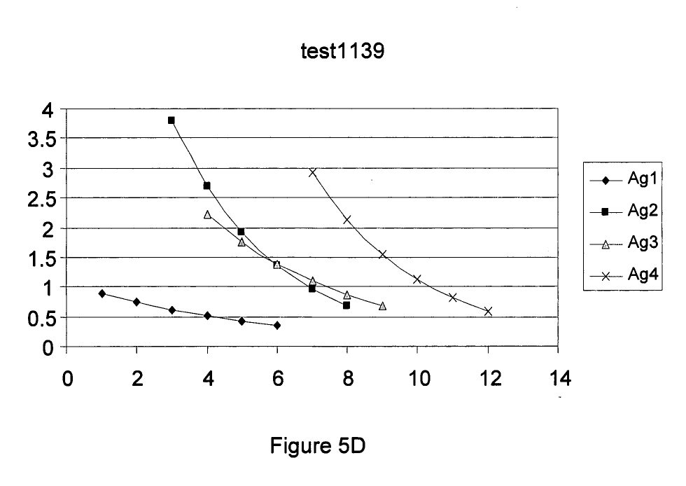

Turning back to the first mode, FIG. 5A is a graph providing results obtained for Ag1-Ag4 with test1139, when the body sample was diluted for all antigens at a single dilution rate which started at 1:8. FIG. 5B provides smoothed results obtained for Ag1-Ag4 with respect to test1139.

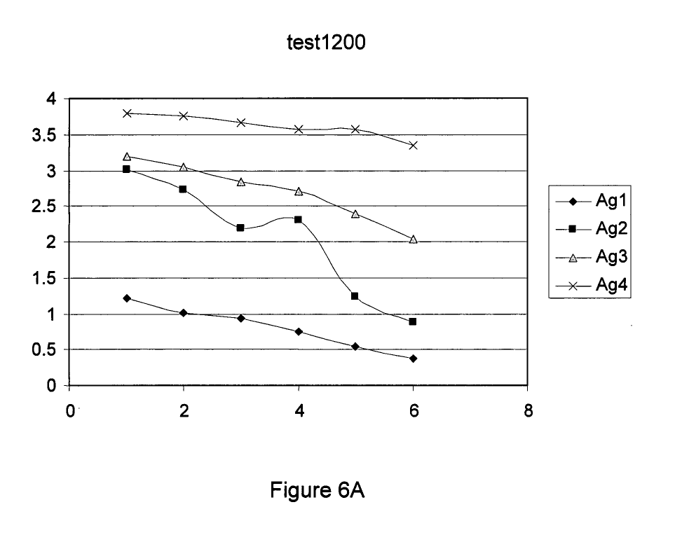

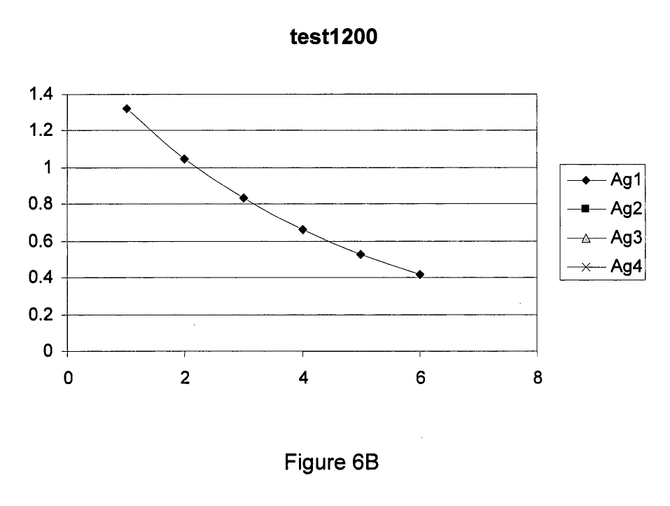

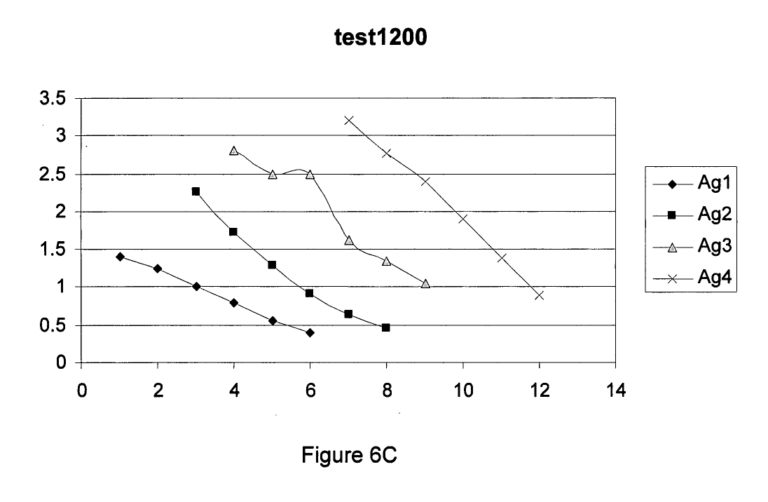

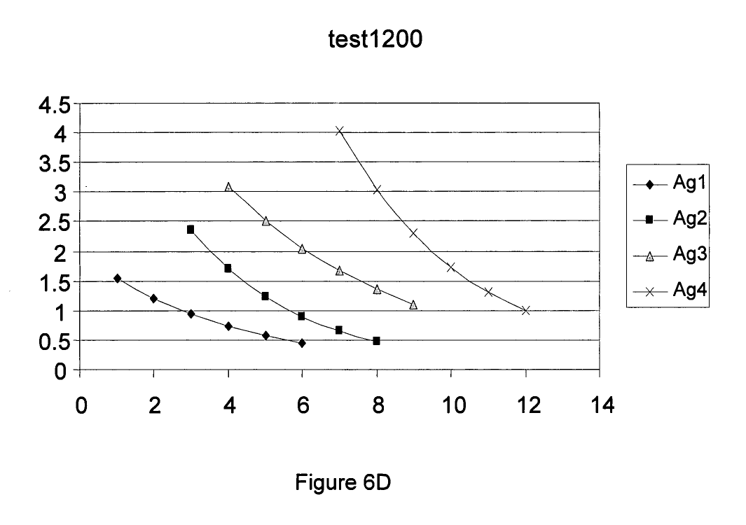

FIG. 6A is a graph showing results obtained for Ag1-Ag4 with test1200, when body sample was diluted for all antigens at a single dilution rate which started at 1:8. FIG. 6B shows smoothed results obtained for Ag1-Ag4 with test1200. Ag2, Ag3 and Ag4 were eliminated due to lack of enough data points.

In FIG. 5B, Ag2 and Ag4 were eliminated due to lack of sufficient data points. In particular, antigens Ag2 and Ag4 have 3 measured points or less with an OD<2.5, which is less than satisfactory to get a linear regression with high certainty. Table 14 provides smoothing results for Ag1-Ag4 of test1139 and test1200. With regards to test1139 insufficient data points for antigens Ag2 and Ag4 are evident.

With respect to test1200, table 14 demonstrate insufficient data points with respect to Ag2, Ag3 and Ag4. For test1200, raw data is described in table 12 and FIG. 6A and FIG. 6B. Ag2, Ag3 and Ag4 have 3 or less measurements with OD<2.5 (after exclusion of outliers) which were insufficient to obtain a linear regression with high certainty.

In order to overcome the shortcomings of using a identical dilution rates for all antigens, body samples were diluted to 4 different starting dilutions. Starting dilution for Ag1 was 1:8, for Ag2 it was 1:32, for Ag3-1:64, for Ag4-1:512. Raw data are summarized in Table 13 and in FIG. 5C for test1139, and FIG. 6C for test1200.

Table 15 provides smoothing results for test1139 and test1200, for antigens Ag1, Ag2, Ag3 and Ag4, with different first dilutions Ag1—1:8, Ag2—1:32, Ag3—1:64, Ag4—1:512. For test1139 and test1200 smoothing results are illustrated in FIGS. 5D and 6D, respectively.

As can be seen in FIGS. 5D and 6D, the tests performed in the second mode achieved perfect smoothing and OD readings within the linear range of the measuring devices for each antigen-antibody complex. These results could thus be further incorporated into the diagnostic algorithm and applications.

According to the above example, in the first mode, there was no common dilution rate series that could have been used for all the antigens in the antigen set Ag1, Ag2, Ag3 and Ag4. Specifically Ag1 and Ag4 did not exhibit such common (or mutual) dilution rate series. Ag1 had a very low signal, while Ag4 had a very high signal (saturated signal OD>2.5, for all dilution rates used, 1-6).

In the second mode, different dilution series were used for each antigens so as to bring the measurable signals into an appropriate OD ranges, e.g. 0>OD<=3 and to allow further processing or to permit the diagnostic methods, diagnostic algorithms and systems of present invention.

In some embodiments, the diagnostic procedures, system and software utilize the predicted values which follows the smoothing procedure, with a different first dilution, as long as the ratio between the dilutions for the different antigens is maintained (see Examples 5-7).

TABLE 12
OD readings obtained for test1139 and test1200 for Ag1-Ag4,
starting dilution rate for all is 1:8

| | | 1 | 2 | 3 | 4 | 5 | 6 |
|---|---|---|---|---|---|---|---|
| sample | antigen | 1:8 | 1:16 | 1:32 | 1:64 | 1:128 | 1:256 |
| test1139 | Ag1 | 0.781 | 0.723 | 0.587 | 0.49 | 0.398 | 0.323 |
| test1139 | Ag2 | 3.794 | 3.588 | 3.136 | 2.601 | 2.042 | 1.455 |
| test1139 | Ag3 | 2.757 | 2.468 | 2.324 | 2.12 | 1.779 | 1.466 |
| test1139 | Ag4 | 3.539 | 3.716 | 3.561 | 3.425 | 3.313 | 3.068 |
| test1200 | Ag1 | 1.215 | 1.008 | 0.928 | 0.747 | 0.534 | 0.37 |
| test1200 | Ag2 | 3.007 | 2.729 | 2.183 | 2.305 | 1.23 | 0.878 |
| test1200 | Ag3 | 3.196 | 3.039 | 2.848 | 2.714 | 2.395 | 2.032 |
| test1200 | Ag4 | 3.791 | 3.766 | 3.665 | 3.572 | 3.577 | 3.351 |

TABLE 13
OD readings for test1139 and test1200, for antigens Ag1, Ag2, Ag3 and Ag4, with different first
dilutions Ag1 - 1:8, Ag2 - 1:32, Ag3 - 1:64, Ag4 - 1:512

| | | 1 | 2 | 3 | 4 | 5 | 6 | 7 | 8 | 9 | 10 | 11 | 12 |
|---|---|---|---|---|---|---|---|---|---|---|---|---|---|
| sample | Antigen | 1:8 | 1:16 | 1:32 | 1:64 | 1:128 | 1:256 | 1:512 | 1:1024 | 1:2048 | 1:4096 | 1:8192 | 1:16384 |
| test1139 | Ag1 | 0.815 | 0.777 | 0.641 | 0.543 | 0.423 | 0.346 | | | | | | |
| test1139 | Ag2 | | | 3.048 | 2.508 | 2.016 | 1.438 | 0.978 | 0.655 | | | | |
| test1139 | Ag3 | | | | 2.043 | 1.82 | 1.463 | 1.154 | 0.875 | 0.628 | | | |
| test1139 | Ag4 | | | | | | | 2.64 | 2.209 | 1.64 | 1.207 | 0.826 | 0.539 |
| test1200 | Ag1 | 1.402 | 1.248 | 1.004 | 0.794 | 0.559 | 0.404 | | | | | | |
| test1200 | Ag2 | | | 2.25 | 1.717 | 1.286 | 0.915 | 0.632 | 0.457 | | | | |
| test1200 | Ag3 | | | | 2.807 | 2.482 | 2.494 | 1.627 | 1.335 | 1.053 | | | |
| test1200 | Ag4 | | | | | | | 3.198 | 2.765 | 2.389 | 1.9 | 1.39 | 0.89 |

TABLE 14
smoothed results for Ag1-Ag4 of test1139 and test1200, first dilution
rate for all antigens is 1:8

| | | 1 | 2 | 3 | 4 | 5 | 6 |
|---|---|---|---|---|---|---|---|
| source | antigen | 1:8 | 1:16 | 1:32 | 1:64 | 1:128 | 1:256 |
| test1139 | Ag1 | 0.828 | 0.690 | 0.575 | 0.479 | 0.399 | 0.333 |
| test1139 | Ag2 | | | | | | |
| test1139 | Ag3 | 2.850 | 2.526 | 2.238 | 1.983 | 1.757 | 1.557 |
| test1139 | Ag4 | | | | | | |
| test1200 | Ag1 | 1.321 | 1.049 | 0.833 | 0.662 | 0.526 | 0.417 |
| test1200 | Ag2 | | | | | | |
| test1200 | Ag3 | | | | | | |
| test1200 | Ag4 | | | | | | |

TABLE 15
smoothed results for Ag1-Ag4 of test1139 and test1200 first dilution rates
Ag1 - 1:8, Ag2 - 1:32, Ag3 - 1:64, Ag4 - 1:512

| | | 1 | 2 | 3 | 4 | 5 | 6 | 7 | 8 | 9 | 10 | 11 | 12 |
|---|---|---|---|---|---|---|---|---|---|---|---|---|---|
| sample | antigen | 1:8 | 1:16 | 1:32 | 1:64 | 1:128 | 1:256 | 1:512 | 1:1024 | 1:2048 | 1:4096 | 1:8192 | 1:16384 |
| test1139 | Ag1 | 0.883 | 0.738 | 0.617 | 0.516 | 0.431 | 0.360 | | | | | | |
| test1139 | Ag2 | | | 3.782 | 2.690 | 1.913 | 1.360 | 0.967 | 0.688 | | | | |
| test1139 | Ag3 | | | | 2.229 | 1.757 | 1.385 | 1.091 | 0.860 | 0.678 | | | |
| test1139 | Ag4 | | | | | | | 2.924 | 2.123 | 1.542 | 1.119 | 0.813 | 0.590 |
| test1200 | Ag1 | 1.554 | 1.206 | 0.936 | 0.727 | 0.564 | 0.438 | | | | | | |
| test1200 | Ag2 | | | 2.347 | 1.699 | 1.230 | 0.890 | 0.645 | 0.467 | | | | |
| test1200 | Ag3 | | | | 3.087 | 2.514 | 2.047 | 1.667 | 1.357 | 1.105 | | | |
| test1200 | Ag4 | | | | | | | 4.019 | 3.035 | 2.292 | 1.731 | 1.307 | 0.987 |

### Example 5

### Classification Rule(s) for Assay Platform

In general, a case-control approach (cancer-healthy) using logistic regression as a tool for building the classification rule was implemented on a training set. The goal of the platform rules and algorithms is to focus on a subset of antigens suitable for use in diagnosis. The inventors developed algorithms and formulas permitting relative accounting of each measurable antigen-autoantibody complex level in order to assign a diagnosis to patients as either “diseased” or “having increased likelihood of being a cancer patient”. Relative accounting of each measurable antigen-autoantibody complex level further encompasses assaying the plurality of antigens at different dilutions rates such that each level is adjusted to an appropriate measurable range. Moreover, built-in solution(s) for addressing personal variability of the autoantibody levels of a diagnosed subject is provided. The platform therefore inter alia, enables not merely accounting for multiple marker presence but also provides cancer diagnostics based on the relative contribution of each antigen-autoantibody levels and takes account of personal variability of antibody profile levels.

Initially, a list of potentially useful antigens is selected for further processing. A training sample of “cancer” and “healthy” subjects is obtained for creating the classification rules.

Measurements of antigen-autoantibody levels were performed for each subject using plurality of consecutive dilutions of each antigen; thereby obtaining a pattern or a set of measurements of antigen-autoantibody levels as a function of the dilution rates. The measurement of optical density (OD) for a given antigen at the given dilution was performed only once (although it may be repeated in some embodiments). Thus, typically there is no mandatory requirement for replicates for a given dilution rate with respect to an antigen.

In some embodiments, quality control measures are taken to increase confidence in the measurements performed. Measurements with OD greater than a predefined threshold (e.g. above 2.5 for certain measurement devices) can be considered as not usable (out of linear range for the detection device) and thus excluded. For each antigen, a dilution rate is established or determined which produced an OD below the predefined OD threshold or within the linear range of the measuring device. Where more that one dilution rate produced an OD below the predefined OD threshold, the minimal can be used.

Optionally, for each antigen, the set of OD readings is processed to produce smoothed/predicted (or processed) OD readings. In some embodiments, linear regression procedure is used to obtain the smoothed/predicted data. The linear regression procedure is applied on a set of OD readings which are deemed by the platform as reliable. By way of non-limiting example, the reliable set of OD readings used for smoothing the data set, are those measurements which exhibit linear reduction of signal proportional to the increased dilution rate, using least square regression. It is also optional to perform weighted least square regression.

The data for a pair <patient, antigen> are also excluded if the squared correlation of the linear regression was below a given threshold (e.g. 0.9). The observed values of ln(OD) are replaced by the smoothed/predicted values. These smoothed values were used for classification.

For at least one pair of antigens, a relative pair-wise dilution ratio is determined. The relative dilution ratio accounts for the ratio between dilution rates. In other words, a measurement or value preset for a diagnosed population and which estimates the proportional relationship between a pair of dilution rates determined for the pair of antigens; each dilution rate produced in the linear range of the measuring device. By way of non-limiting example, let x₁ and x₂ be a pair of dilution rates of Ag₁ and Age respectively; the pair of dilution rates producing a pair of OD readings within the linear range of measuring device. Therefore, the relative dilution ratio can be defined as x₁/x₂.

The relative dilution rate is used thereafter for analysis of either cancer or healthy subjects (in the training set). The measurements of antigen-autoantibody levels of the pair of antigens (obtained from a subject) are assumed to be and operable such that the dilution ratio is maintained between the measurement pair and across the population of subjects.

By way of non-limiting example, the relative dilution rate can be used as follows. Following the obtaining of measurement of an antigen-autoantibody complex level (e.g of Ag1) at a particular dilutions rate x1, the dilution ratio (e.g. x1/x2) is used to predict or theoretically estimate the dilution rate in which to obtain the antigen-autoantibody complex level of Ag2. If the antigen-autoantibody complex level at the predicted dilution rate is not available (such as for reasons of technical limitation/saturation of the signal at the predicted/theoretical dilution rate) the smoothed data can be used to predict the antigen-autoantibody complex level at the predicted/theoretical dilution rate. Such prediction can take the form of extrapolation procedure (such use of relative dilution ratio is used similarly in the diagnostic methods and applications discussed below).

This procedure of obtaining the OD readings is repeated until all the smoothed antigen-autoantibody complex levels are ascertained with respect to each subject in both “cancer” and “healthy” groups and each potentially useful antigens.

The set of all pair <antigen, smoothed OD reading> of a subject in either “cancer” and “healthy” groups, is processed by a logistic regression procedure. Logistic regression procedure can be used to measure the relative contribution of the smoothed OD readings of each autoantibody-antigen complex. The results comprised, therefore, a plurality of possible subsets of antigens and their relative contribution to the occurrence of cancer; these were obtained under the pre-set relative dilution ratio constraints.

The results are ordered by the area under the receiver operating curve (AUC). If the specific combination posed some limitations or dissatisfaction on acceptable sensitivity or specificity, the partial AUC (pAUC) can be used for ordering the results. i.e. imposing a pre-determined limitation of sensitivity or specificity. Some antigen subsets of best combinations are then presented for the further no-formal expert analysis.

Logistic regression procedure can comprised the following:
a) the “best n subset” Ag₁, Ag₂, and Ag₃ . . . . Agₙ;
b) the linear test function of the form z=b₀+b₁o₁+b₂o₂+b₃o₃+ . . . +bₙoₙ where o₁, o₂, o₃ and oₙ are smoothed logarithm of optical density of the n antigens Ag₁, Ag₂, Ag₃ . . . , Agₙ for given dilutions (may be different for different antigens), and b₀, b₁, b₂, b₃ and bₙ are coefficients or factors of the appropriate logistic regression model used;
c) the overall threshold Z₀, such that if z>Z₀ the woman/subject be classified as “cancer” and if z Z₀, the woman/subject would be classified as “healthy”.

In some embodiments, the function z=f(relative contribution parameters) is used in the logistic regression model; In some specific embodiments, the function is a polynomial function in the form of z=b₀+b₁o₁+b₂o₂+b₃o₃+ . . . +bₙoₙ; In other specific embodiments, the function is z=ln(b₀+b₁o₁+b₂o₂+b₃o₃+ . . . +bₙoₙ); z=ln(b₀)+ln(b₁o₁)+ln(b₂o₂)+ln(b₃o₃)+ . . . +ln(bₙoₙ); and the like.

The linear classifier based on the preferred or best selected antigen subset was validated on independent (examination) sample of “cancer” and “healthy” subjects to validate the stability of the subset in it's ability to predict the health status of the unknown population. The set of coefficients b₀, b₁, b₂, b₃ and bₙ which being validated as maintaining stability of the subset and it's ability to predict the health status of the unknown population can be used as the relative contribution factors for the further diagnostic methods and applications in the clinical settings. b₀ is typically a free coefficient. The values or coefficients can be maintained in a relative contribution factor matrix as fixed or constants for providing the diagnostic applications.

The above classification rule and procedures were used in Examples 6 and 7 below.

### Example 6

### Determining Subsets of Antigens for Diagnostic Application

Plasma samples were obtained with consent from 40 cancer patients having a biopsy positive for breast cancer. Plasma sample were obtained with consent from 42 healthy subjects negative for breast cancer.

By way of non-limiting exemplification, Table 16 provides the list of antigens which were used herein below.

Relative pair-wise dilution ratio was further determined for the above antigens by serial dilution of at least 5 samples starting from 1:10 as previously described.

Representative dilution rate for each antigen was determined in the present example as the first/minimal dilution rate in the series of at least 6 OD readings that were responsive to dilutions for the samples i.e. at least 6 consecutive OD readings which were not saturated and exhibited substantially logarithmic dependency between the measured signal and dilution rate used.

Relative dilution ratio between antigen or pairs of antigens was obtained from a series of the smoothed OD readings obtained by using different dilution rates.

The relative dilution ratio was obtained from the first dilution rate in the corresponding OD series of each antigen in the pair. The relative dilution ratio is maintained as a constraint for the processing of the OD readings of each subject in the cancer or healthy groups.

It should be noted that the present invention can be similarly be used by a representative dilution rate from at least 3, 4, 5, 6, 7, 8, 9 or more successive OD readings. A representative dilution rate in the series is selected for further processing.

Table 16 further provides the representative dilution rate of plasma samples for each antigen (chosen to be the first in a OD series).

Each sample of either the cancer subjects and the healthy subjects was diluted to the first dilution rate or point detailed in table 16 and ELISA was performed. ln(OD) was calculated and first dilution OD for each antigen used. In general, the pair-wise dilution ratio determined between any one antigen of the group: {LDPe002, LDPe012, LDPe016, LDPe039, LDPe066, LDPe069, LDPe070 and LDPe071} to any one antigen of the group: {LDPr041, LDPr076, LDPr077, LDPr078, LDPr079, LDPr095} was 0.5. In particular, the pair-wise dilution ratio of LDPe071 to LDPr041 is 0.5. The pair-wise dilution ratios was maintained during obtaining of the measured OD data.

TABLE 16
List of antigens used in example 6 and first dilution rate for each

| | First dilution |
|---|---|
| Ag | rate |
| LDPe002 | 1:32 |
| LDPe012 | 1:32 |
| LDPe016 | 1:32 |
| LDPe039 | 1:32 |
| LDPe066 | 1:32 |
| LDPe069 | 1:32 |
| LDPe070 | 1:32 |
| LDPe071 | 1:32 |
| LDPr041 | 1:64 |
| LDPr076 | 1:64 |
| LDPr077 | 1:64 |
| LDPr078 | 1:64 |
| LDPr079 | 1:64 |
| LDPr095 | 1:64 |

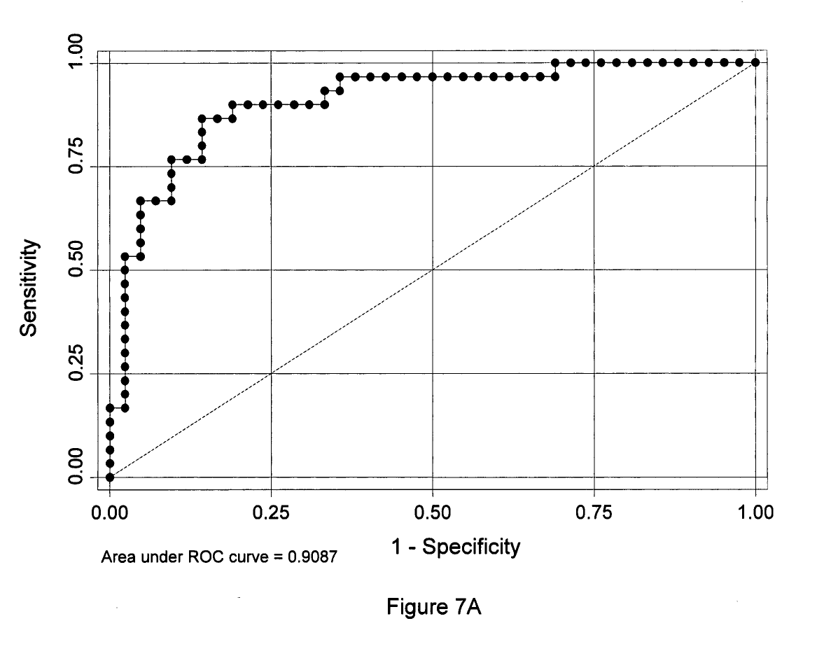

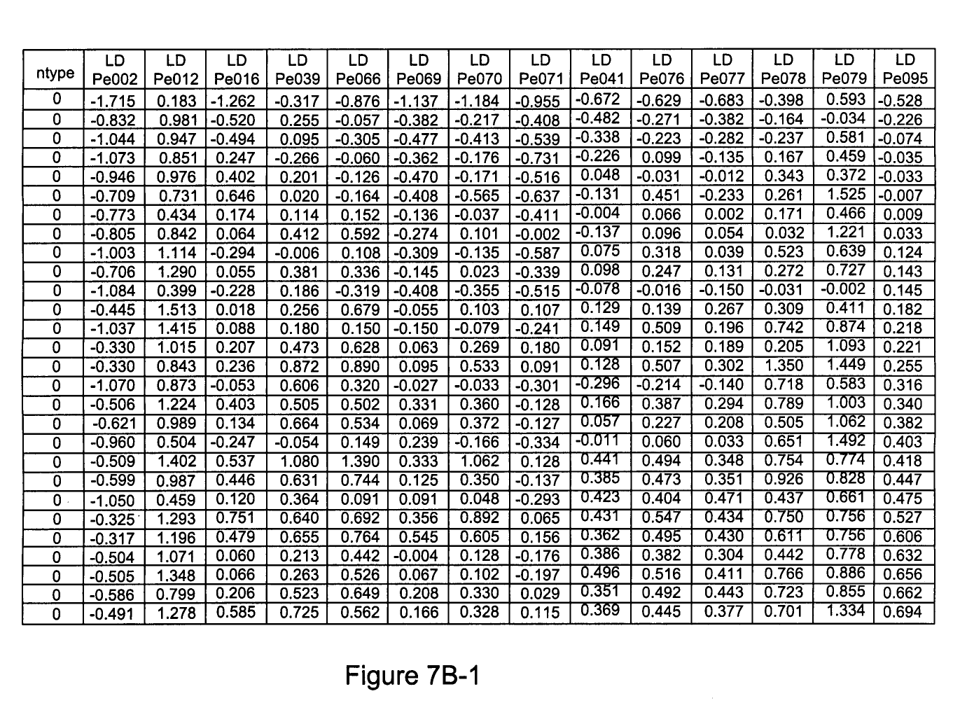

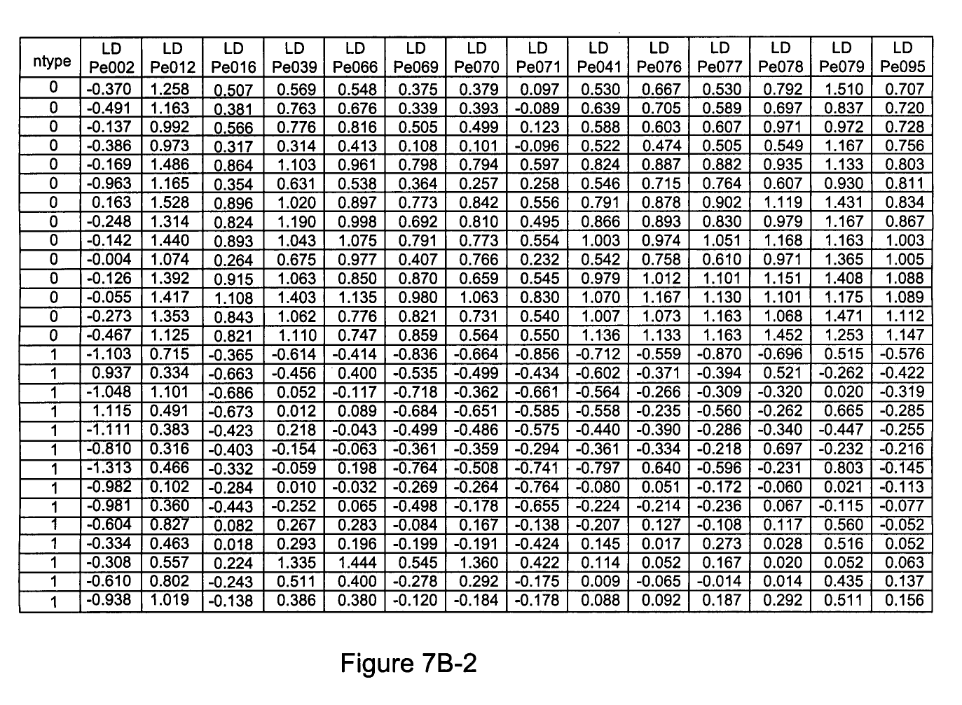

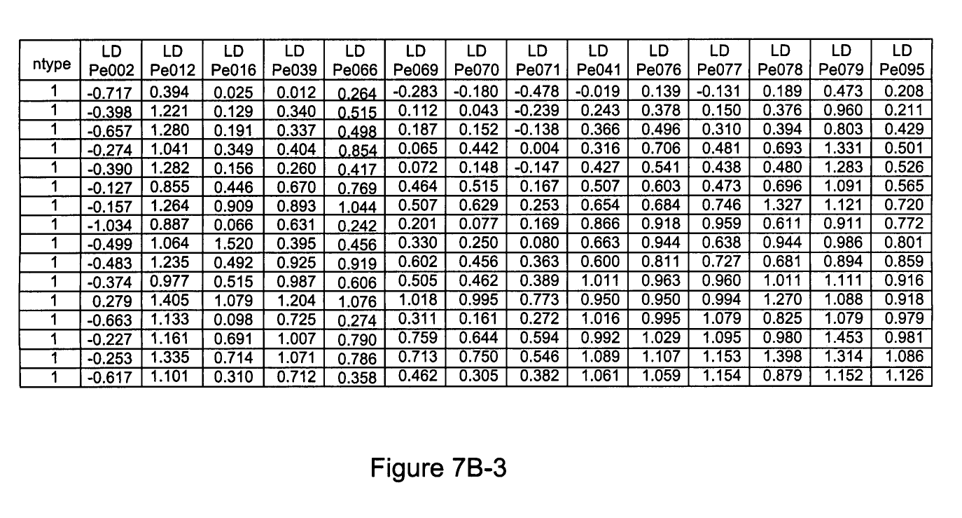

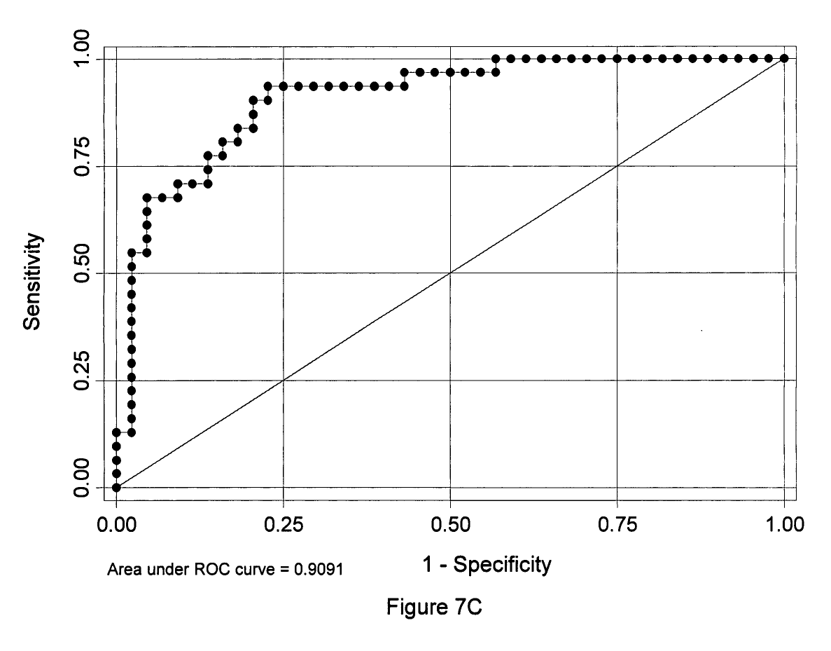

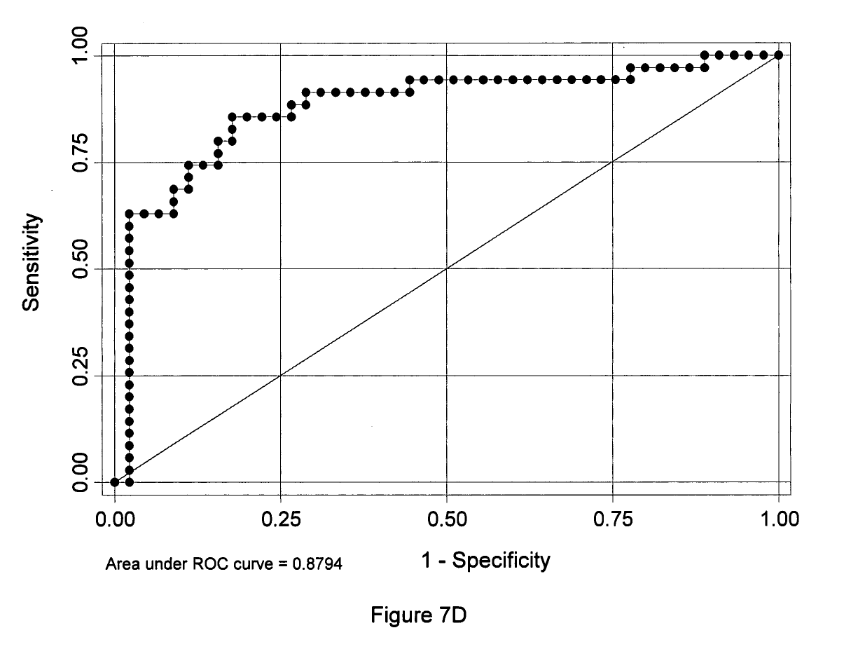

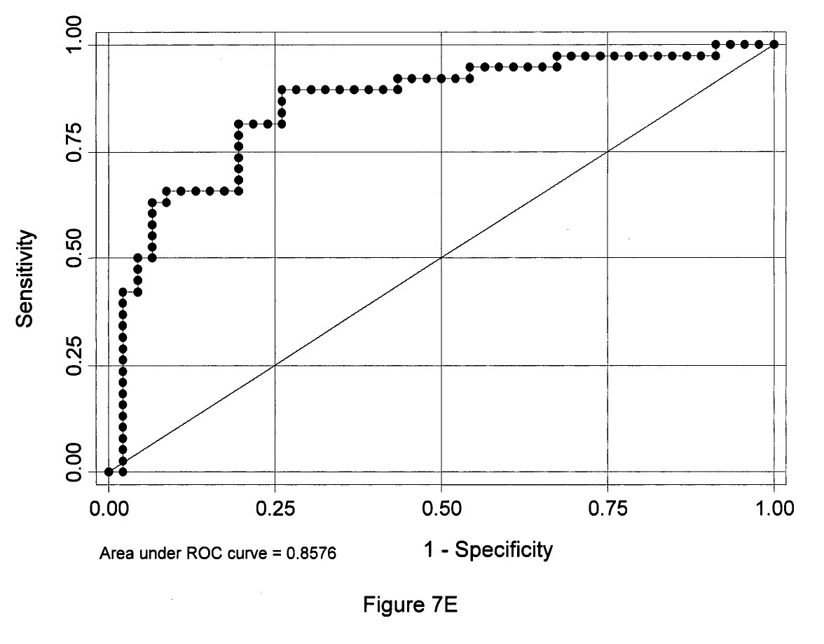

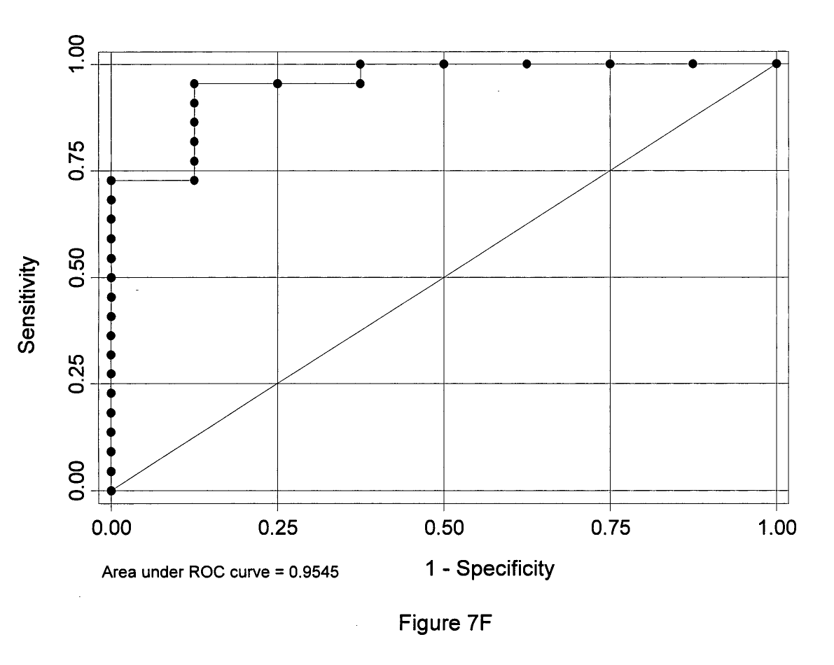

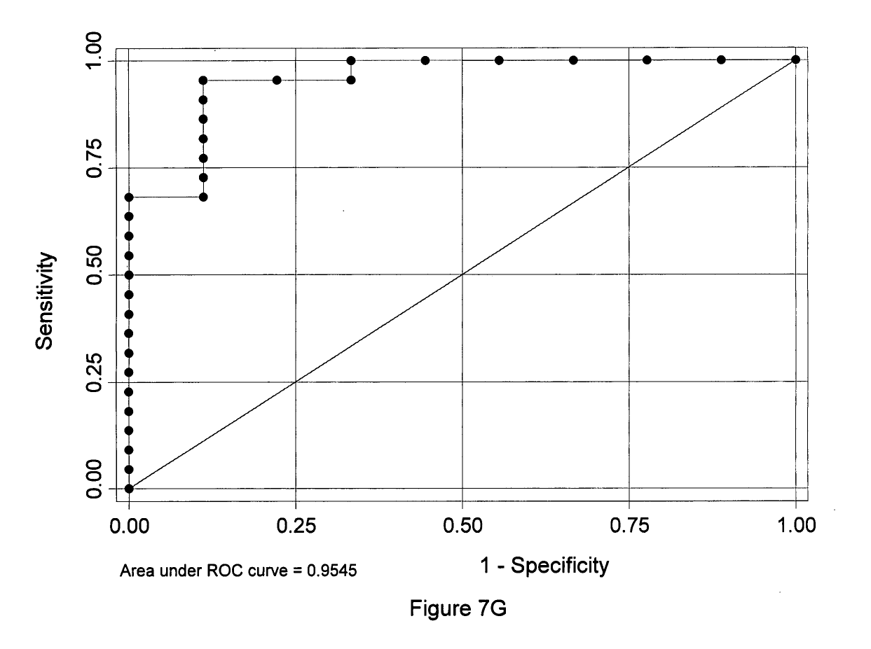

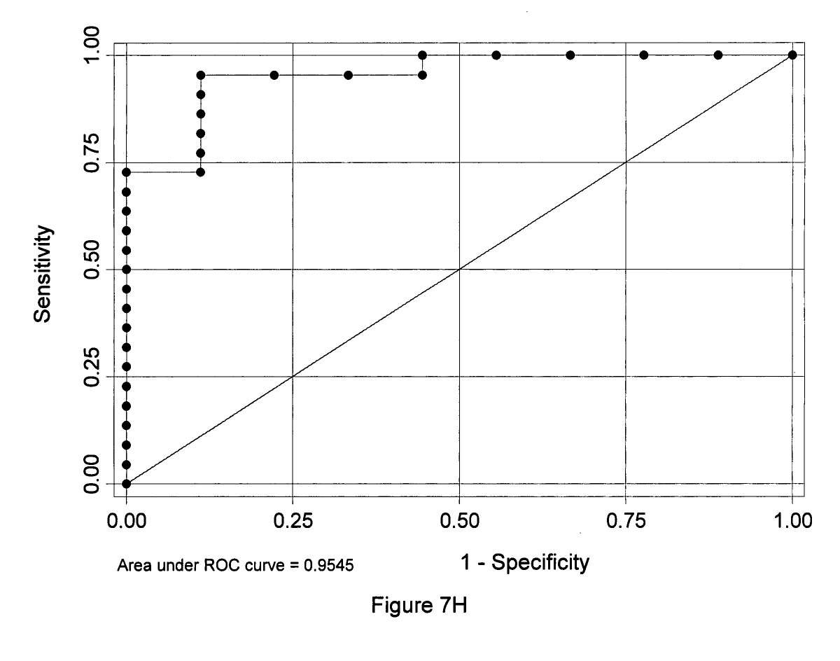

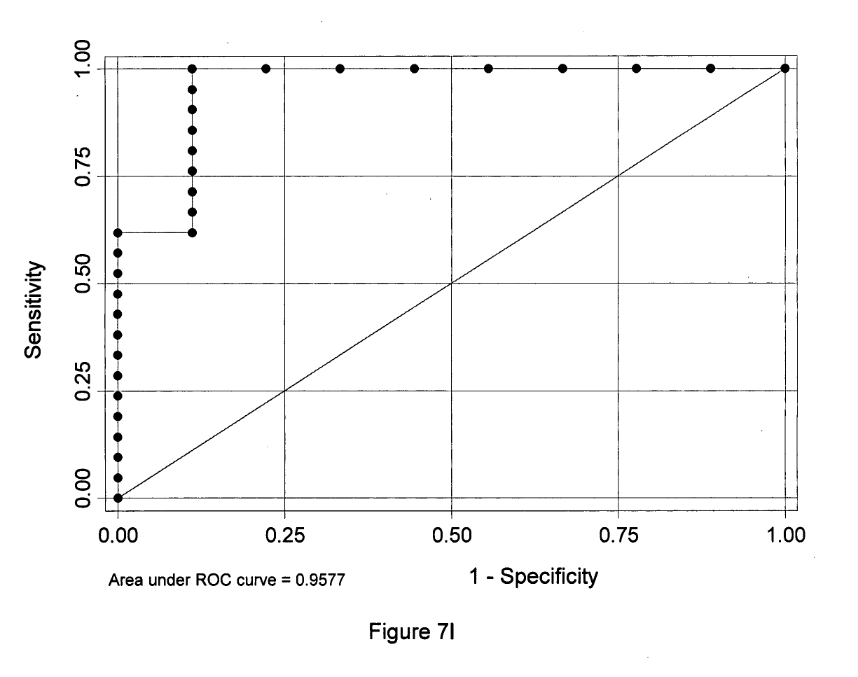

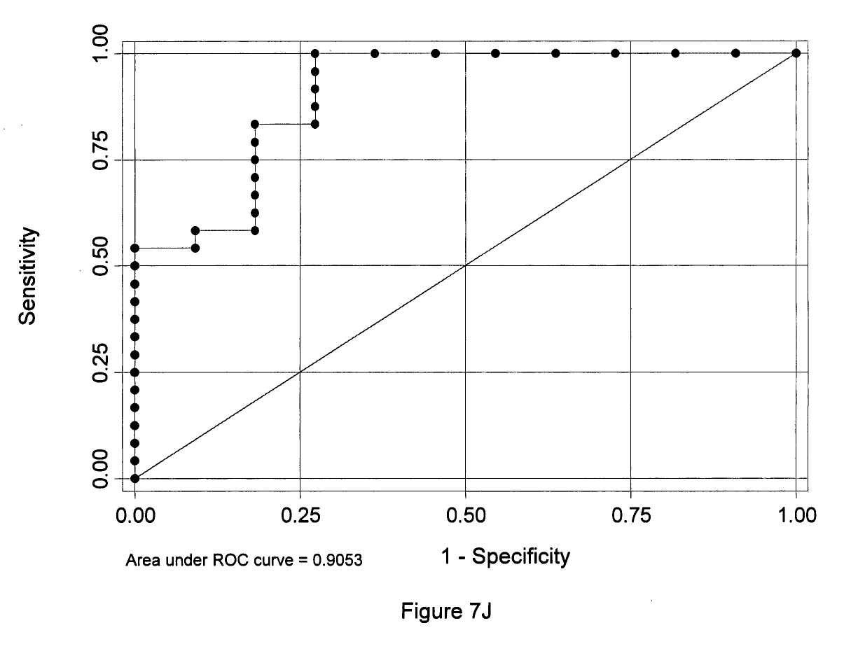

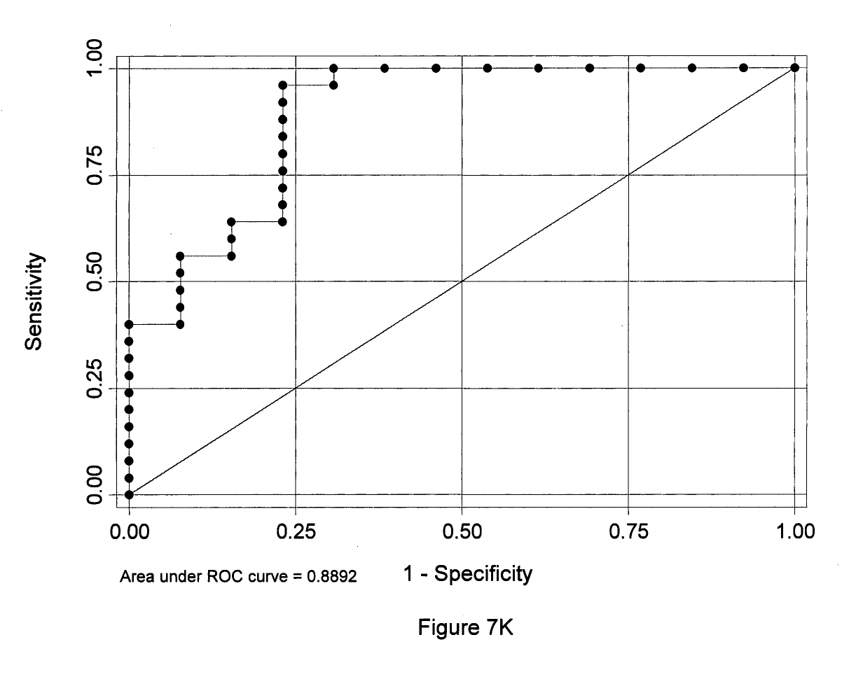

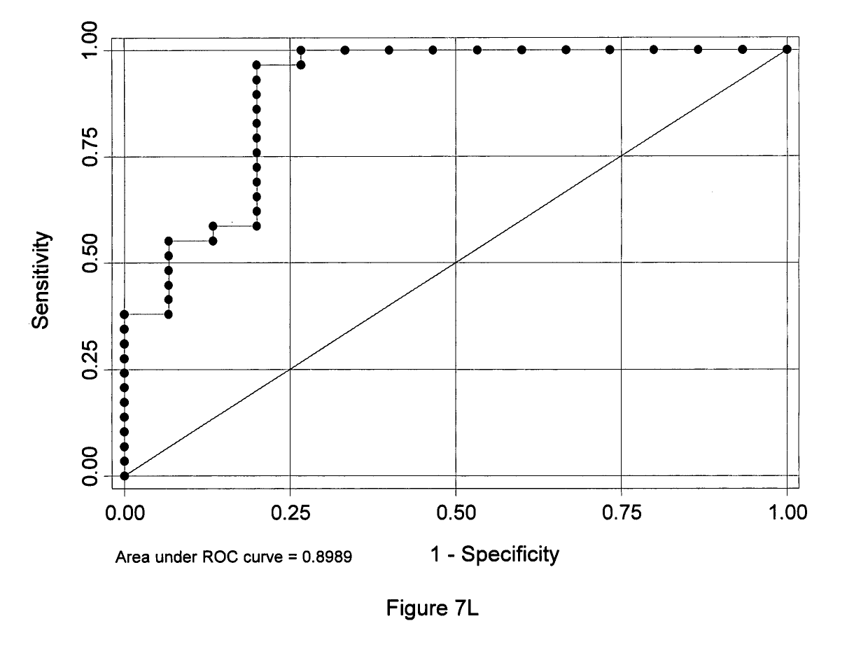

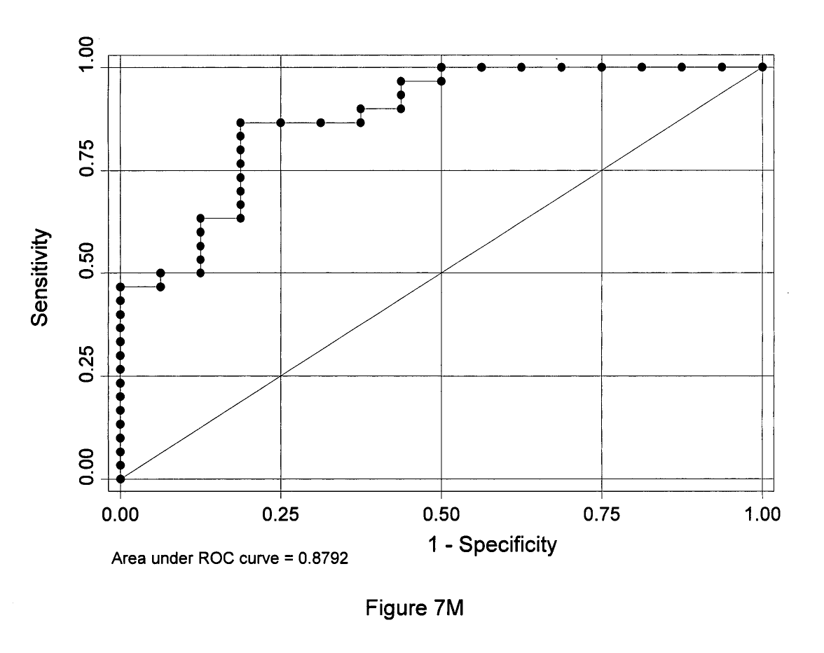

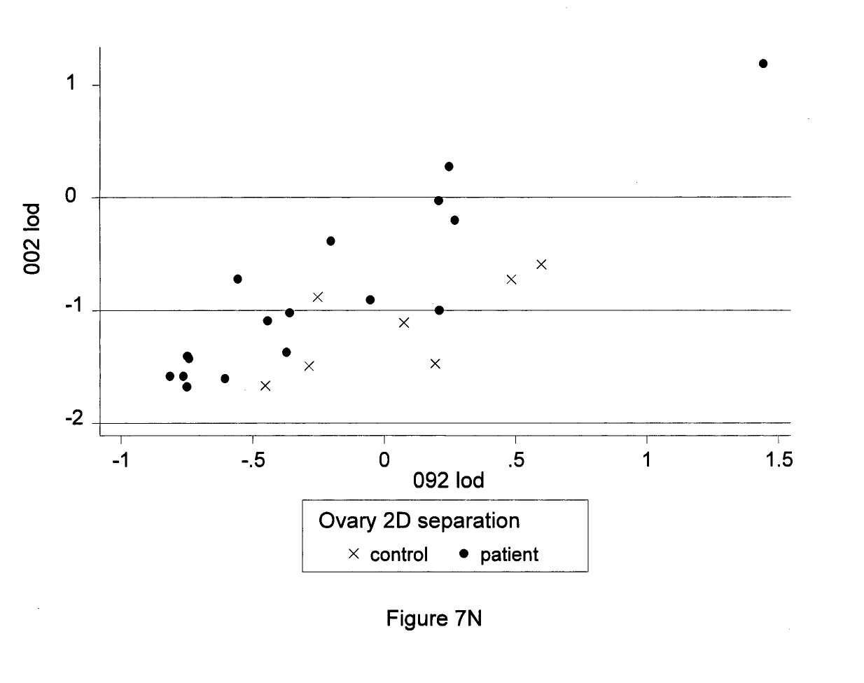

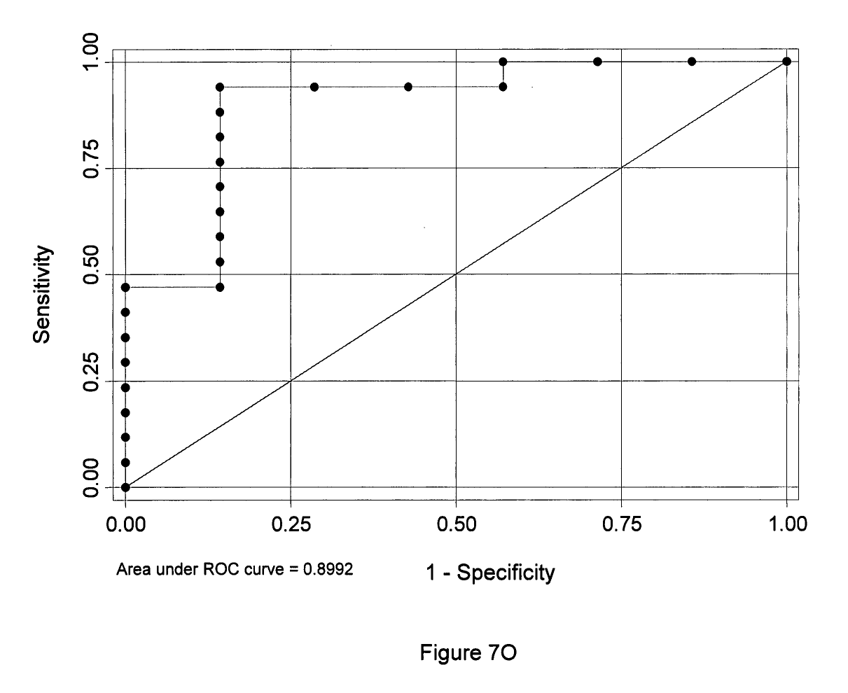

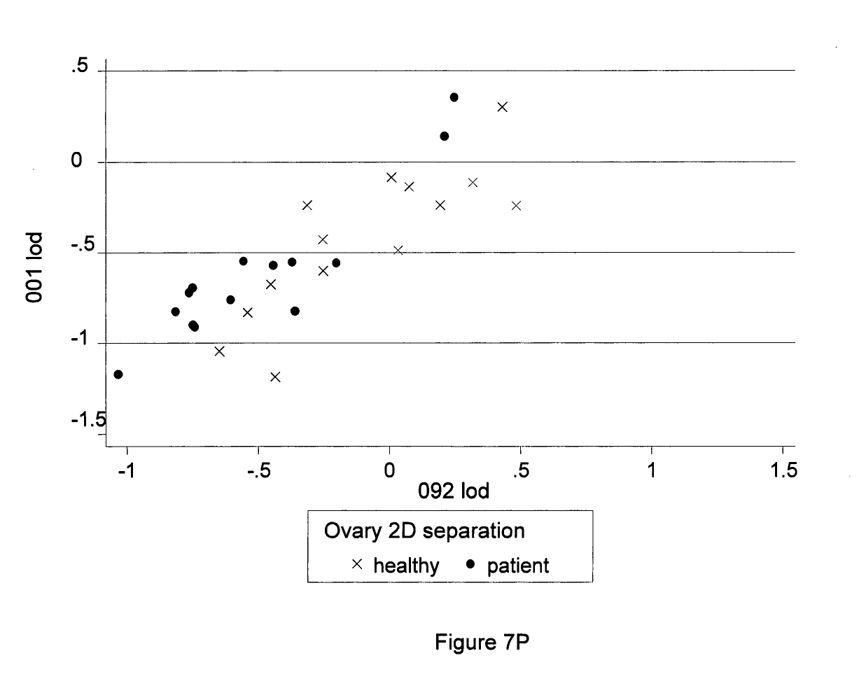

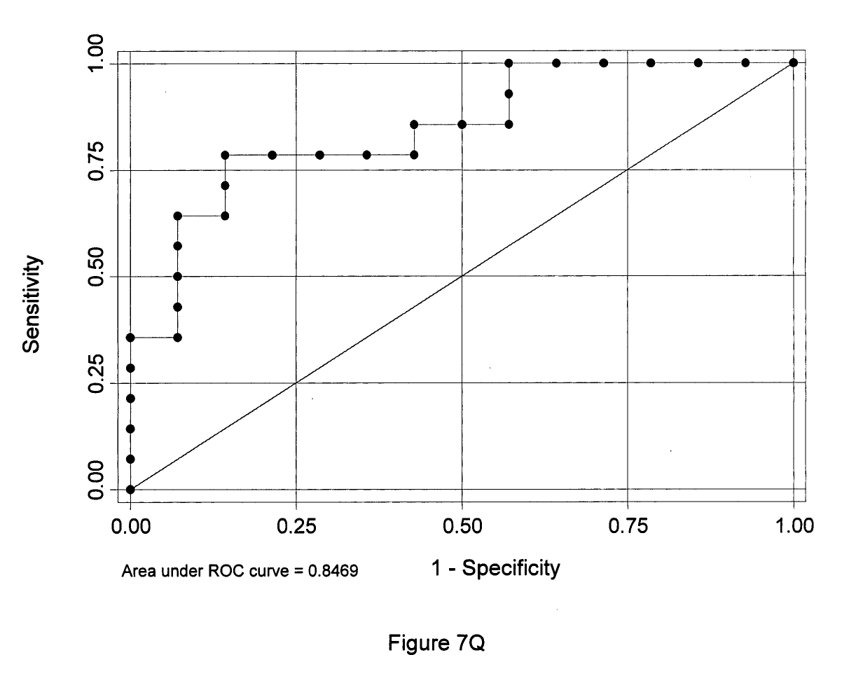

Table 17 (FIG. 7B) details the ln(OD) results for the first dilution rate of each antigen and each sample. Row ntype “0” represents healthy samples, and row ntype “1” represents cancer sample. Logistic regression of the above antigen combination was performed as described above, and ROC analysis determined the AUC of the above samples set, as shown in FIG. 7A.

Using identical procedure additional subsets of antigens were identified as permitting both statistical separation between diseased/cancer subjects and healthy controls.

In particular, utilization of a subset comprising 13 antigens resulted with an area under ROC curve of 0.9091 (Table 18, FIG. 7C); a subset comprising 12 antigens resulted with an area under ROC curve of 0.8794 (Table 19, FIG. 7D); a subset comprising 11 antigens resulted with an area under ROC curve of 0.8576 (Table 20, FIG. 7E); a subset comprising 10 antigens resulted with an area under ROC curve of 0.9545 (Table 21, FIG. 7F); a subset comprising 9 antigens resulted with an area under ROC curve of 0.9545 (Table 22, FIG. 7G); a subset comprising 8 antigens resulted with an area under ROC curve of 0.9545 (Table 23, FIG. 7H); a subset comprising 7 antigens resulted with an area under ROC curve of 0.9577 (Table 24, FIG. 7I); a subset comprising 6 antigens resulted with an area under ROC curve of 0.9053 (Table 25, FIG. 7J); a subset comprising 5 antigens resulted with an area under ROC curve of 0.8892 (Table 26, FIG. 7K); a subset comprising 4 antigens resulted with an area under ROC curve of 0.8989 (Table 27, FIG. 7L); and a subset comprising 3 antigens resulted with an area under ROC curve of 0.8792 (Table 28, FIG. 7M);

TABLE 18
A subset of 13 antigens and first dilution rate for each

| | First dilution |
|---|---|
| Ag | rate |
| LDPe002 | 1:32 |
| LDPe012 | 1:32 |
| LDPe016 | 1:32 |
| LDPe039 | 1:32 |
| LDPe066 | 1:32 |
| LDPe069 | 1:32 |
| LDPe070 | 1:32 |
| LDPe071 | 1:32 |
| LDPr041 | 1:64 |
| LDPr076 | 1:64 |
| LDPr078 | 1:64 |
| LDPr079 | 1:64 |
| LDPr095 | 1:64 |

TABLE 19
A subset of 12 antigens and first dilution rate for each

| | First dilution |
|---|---|
| Ag | rate |
| LDPe002 | 1:32 |
| LDPe016 | 1:32 |
| LDPe039 | 1:32 |
| LDPe066 | 1:32 |
| LDPe069 | 1:32 |
| LDPe070 | 1:32 |
| LDPe071 | 1:32 |
| LDPr041 | 1:64 |
| LDPr076 | 1:64 |
| LDPr078 | 1:64 |
| LDPr079 | 1:64 |
| LDPr095 | 1:64 |

TABLE 20
A subset of 11 antigens and first dilution rate for each

| | First dilution |
|---|---|
| Ag | rate |
| LDPe002 | 1:32 |
| LDPe039 | 1:32 |
| LDPe066 | 1:32 |
| LDPe069 | 1:32 |
| LDPe070 | 1:32 |
| LDPe071 | 1:32 |
| LDPr041 | 1:64 |
| LDPr076 | 1:64 |
| LDPr078 | 1:64 |
| LDPr079 | 1:64 |
| LDPr095 | 1:64 |

TABLE 21
A subset of 10 antigens and first dilution rate for each

| | First dilution |
|---|---|
| Ag | rate |
| LDPe001 | 1:32 |
| LDPe002 | 1:32 |
| LDPe008 | 1:32 |
| LDPe012 | 1:32 |
| LDPe016 | 1:32 |
| LDPe039 | 1:32 |
| LDPe066 | 1:32 |
| LDPe069 | 1:32 |
| LDPe070 | 1:32 |
| LDPe071 | 1:32 |

TABLE 22
A subset of 9 antigens and first dilution rate for each

| | First dilution |
|---|---|
| Ag | rate |
| LDPe001 | 1:32 |
| LDPe002 | 1:32 |
| LDPe008 | 1:32 |
| LDPe012 | 1:32 |
| LDPe016 | 1:32 |
| LDPe039 | 1:32 |
| LDPe066 | 1:32 |
| LDPe070 | 1:32 |
| LDPe071 | 1:32 |

TABLE 23
A subset of 8 antigens and first dilution rate for each

| | First dilution |
|---|---|
| Ag | rate |
| LDPe001 | 1:32 |
| LDPe002 | 1:32 |
| LDPe008 | 1:32 |
| LDPe012 | 1:32 |
| LDPe016 | 1:32 |
| LDPe066 | 1:32 |
| LDPe070 | 1:32 |
| LDPe071 | 1:32 |

TABLE 24
A subset of 7 antigens and first dilution rate for each

| | First dilution |
|---|---|
| Ag | rate |
| LDPe001 | 1:32 |
| LDPe002 | 1:32 |
| LDPe008 | 1:32 |
| LDPe012 | 1:32 |
| LDPe070 | 1:32 |
| LDPe071 | 1:32 |
| LDPe072 | 1:32 |

TABLE 25
A subset of 6 antigens and first dilution rate for each

| | First dilution |
|---|---|
| Ag | rate |
| LDPe002 | 1:32 |
| LDPe008 | 1:32 |
| LDPe012 | 1:32 |
| LDPe070 | 1:32 |
| LDPe071 | 1:32 |
| LDPe072 | 1:32 |

TABLE 26
A subset of 5 antigens and first dilution rate for each

| | First dilution |
|---|---|
| Ag | rate |
| LDPe002 | 1:32 |
| LDPe012 | 1:32 |
| LDPe070 | 1:32 |
| LDPe071 | 1:32 |
| LDPe072 | 1:32 |

TABLE 27
A subset of 4 antigens and first dilution rate for each

| | First dilution |
|---|---|
| Ag | rate |
| LDPe012 | 1:32 |
| LDPe070 | 1:32 |
| LDPe071 | 1:32 |
| LDPe072 | 1:32 |

TABLE 28
A subset of 3 antigens and first dilution rate for each

| | First dilution |
|---|---|
| Ag | rate |
| LDPe012 | 1:32 |
| LDPe071 | 1:32 |
| LDPe072 | 1:32 |

### Example 7

### Determining Sets of Antigens for Ovarian Cancer Diagnostic Application

Plasma samples were obtained from 20 epithelial ovarian cancer patients having a verified pathology. Plasma sample were also obtained from 20 healthy subjects. Informed consent was obtained from all subjects.

Table 29 provides the list of antigens which were used herein below. Relative dilution ratio was determined for the antigens by serial dilution of at least 5 samples starting from 1:5 as previously described.

TABLE 29
A subset of 3 antigens and first dilution
rate for each for ovarian diagnostics

| | First Dilution |
|---|---|
| Antigen | rates |
| LDPe001 | 1:8 |
| LDPe002 | 1:8 |
| LDPe092 | 1:8 |

The representative dilution rate for each antigen was determined in the present example as the first/minimal dilution rate in a series of at least 6 OD readings that were responsive to dilutions for the samples, as previously explained.

Table 29 further provides the first dilution rate of plasma sample for each antigen. ln(OD) was calculated and first dilution OD for each antigen used. Table 30 details the ln(OD) results for the first dilution rate of each antigen and each sample. Ntype “0” represents healthy samples, and ntype “1” represents patients sample. A total of 7 patients and 17 healthy were tested.

Logistic regression of the above antigen combination LDPe002 (SEQ ID no. 9) and LDPe092 (SEQ ID no. 16) was performed as described above, and ROC analysis determined the AUC of the above samples set, as shown in FIG. 7N.

TABLE 30
ln(OD) results for the first dilution
rate of each antigen and each sample.

| ntype | LDPe002 | LDPe092 |
|---|---|---|
| 0 | −1.672 | −0.453 |
| 0 | −1.494 | −0.285 |
| 0 | −1.476 | 0.195 |
| 0 | −1.109 | 0.075 |
| 0 | −0.882 | −0.251 |
| 0 | −0.723 | 0.484 |
| 1 | −1.680 | −0.751 |
| 1 | −1.606 | −0.605 |
| 1 | −1.587 | −0.814 |
| 1 | −1.584 | −0.763 |
| 1 | −1.430 | −0.743 |
| 1 | −1.409 | −0.749 |
| 1 | −1.372 | −0.370 |
| 1 | −1.093 | −0.443 |
| 1 | −1.020 | −0.360 |
| 1 | −1.000 | 0.211 |
| 1 | −0.909 | −0.051 |
| 1 | −0.722 | −0.555 |
| 1 | −0.385 | −0.201 |
| 1 | −0.029 | 0.208 |
| 1 | 0.270 | 0.249 |
| 1 | 1.179 | 1.444 |

ntype “0” represents healthy samples, and ntype “1” represents cancer sample

FIG. 7N shows results obtained for the first minimal dilution for LDPe002 and LDPe092 for 7 patients and 17 healthy subjects, FIG. 7O shows the AUC calculation for the above data as 0.90%.

Using identical procedure additional subsets of antigens were identified as permitting both statistical separation between diseased/cancer subjects and healthy controls.

Logistic regression of the above antigen combination LDPe001 (SEQ ID no. 8) and LDPe092 (SEQ ID no. 16) was performed as described above, and ROC analysis determined the AUC of the above samples set, as shown in FIG. 7P.

TABLE 31
ln(OD) results for the first dilution
rate of each antigen and each sample.

| ntype | LDPe001 | LDPe092 |
|---|---|---|
| 0 | −1.185 | −0.436 |
| 0 | −1.043 | −0.649 |
| 0 | −0.831 | −0.542 |
| 0 | −0.676 | −0.453 |
| 0 | −0.603 | −0.251 |
| 0 | −0.487 | 0.034 |
| 0 | −0.427 | −0.254 |
| 0 | −0.242 | 0.484 |
| 0 | −0.238 | 0.195 |
| 0 | −0.238 | −0.314 |
| 0 | −0.138 | 0.075 |
| 0 | −0.113 | 0.318 |
| 0 | −0.084 | 0.009 |
| 0 | 0.301 | 0.430 |
| 1 | −1.170 | −1.033 |
| 1 | −0.909 | −0.743 |
| 1 | −0.900 | −0.749 |
| 1 | −0.827 | −0.814 |
| 1 | −0.823 | −0.360 |
| 1 | −0.760 | −0.605 |
| 1 | −0.720 | −0.763 |
| 1 | −0.694 | −0.751 |
| 1 | −0.572 | −0.443 |
| 1 | −0.558 | −0.201 |
| 1 | −0.554 | −0.370 |
| 1 | −0.547 | −0.555 |
| 1 | 0.141 | 0.211 |
| 1 | 0.354 | 0.249 |

ntype “0” represents healthy samples, and ntype “1” represents cancer sample

FIG. 7P shows results obtained for the first/minimal dilution for LDPe001 and LDPe092 for 14 patients and 14 healthy subjects.

FIG. 7Q shows the AUC calculation for the above data as 0.85%.

### Example 8

### Diagnostic Methods and Applications

Methods and applications for assigning a diagnosis to a subject being assessed for the presence of cancer utilizes input parameters previously determined. The methods and applications thus utilize the knowledge of an established set of antigens; relative dilution ratios of antigen pairs of the set and/or the antigen representative dilution rate; and the relative contribution factor of each of said antigens.

The methods and applications permit a determination whether a diagnosed subject should be assigned with a diagnosis of being afflicted with cancer or whether the subject has an increased likelihood of being afflicted with cancer.

Initially, a body sample is obtained from the subject to be diagnosed. Generally, the sample is contacted with a subset of antigens previously determined as suitable for the diagnostic procedure.

The subset of antigens, for example, Ag₁, Ag₂ and Ag₃ are contacted with the body sample of the subject and thereafter analyzed for at least 3 OD readings (optionally 6) in 3 or more dilution rates per antigen.

The assay comprises contacting said body sample at representative dilution rates to form complexes with the autoantibodies within the plurality of sample aliquots. Typically, sequential dilutions starting with the minimal one are performed. As previously explained, the dilution rates (including the minimal dilution rate) can be different for different antigens. A plurality of sample aliquots in different dilution rates is obtained.

Quality control procedures are applied for the measured data for each antigen-antibody complexes level separately. Measurements with OD greater than a predefined threshold (e.g. above 2.5 for certain measuring devices) can be considered as not usable (out of linear range for the detection device) and thus excluded. In case the data for does not pass the quality control phase, the test is deemed as “technically unsuccessful”. In order to conclude the diagnosis repeated measurements should take place followed, again, by quality assurance procedures.

Where quality control procedure was followed and, the measurements for each antigen was verified in that respect, data is smoothed as was described hereinabove; thereby obtaining smoothed/predicted OD readings. Optionally, the diagnostic determination is determined on the basis of the smoothed or predicted OD readings. In some embodiments, the relative dilution ratio is maintained between the antigens. The set of OD readings/smoothed OD readings/predicted OD readings representing the antigen-autoantibody complex levels are thus obtained. e.g., o₁, o₂, o₃ and oₙ optical densities of the n antigens Ag₁, Ag2, Ag3 . . . , Agₙ.

The value of relative contribution parameters of each of said antigen-antibody complexes levels to the presence of cancer is calculated by adjusting each of said antigen-antibody complex levels in accordance with the predetermined relative contribution factor, previously determined or fixated. In some embodiments, adjustment takes the form of multiplying PARAMᵢ=oᵢbᵢ,1≧i≧n, where oᵢ an antigen-antibody complexes level of Agᵢ, and bᵢ is the relative contribution factor characterizing antigen Agᵢ. In other embodiments, PARAMᵢ=ln(oᵢbᵢ),1≧i≧n.

The discriminant/test function (x) is then calculated, the input of which are the relative contribution parameters. The value (x) will be compared with a predetermined cut-point Z. In some embodiments, the discriminant/test function (x)=Σᵢ₌₁ⁿ PARAMᵢ or (x)=b₀+b₁o₁+b₂o₂+b₃o₃+ . . . +bₙoₙ

A diagnosed subject will be assigned or classified as “having increased likelihood of being afflicted with cancer” if x>Z, and “healthy” otherwise.

In the following example, the breast surgeon is requesting a diagnostic set of antigens, which will allow high sensitivity and moderate specificity. In this mode, a surgeon can eliminate a large portion of the unnecessary biopsies done at present.

A population of suspected women who were thus scheduled for breast biopsy or surgery gave a plasma sample with informed consent. Each of the women had also allowed accessibility to the biopsy results. The plasma samples were divided randomly into two sets of samples—a training set and a validation set.

A total of 106 samples served as the training set. A set of antigens was proposed and the best subset was chosen according to the methods disclosed above and under the surgeon's specifications (high sensitivity in order to not miss patients, and moderate specificity, in order to send at least 50% of the healthy population for monitoring only without the need to have a biopsy).

A subset containing 8 antigens resulted in the following relative contribution factor provided in table 32.

TABLE 32
Subset of 8 antigens used for diagnosis, their relative dilution rate
and the relative contribution factors calculated by logistic regression

| | Relative | Relative |
|---|---|---|
| | dilution | Contribution |
| Antigen | rate | Factor |
| LDPe002 | 1:32 | 6.813902 |
| LDPe012 | 1:32 | −3.984125 |
| LDPe016 | 1:32 | −3.825265 |
| LDPe066 | 1:32 | 6.346947 |
| LDPe070 | 1:32 | −6.585065 |
| LDPr077 | 1:64 | 11.45633 |
| LDPr095 | 1:64 | −8.415792 |
| LDPe071 | 1:32 | −3.317258 |
| Free coefficient | | 6.708538 |
| Z(cut off) | | −1.16 |

Using this set of antigens in an assay performed at the specific representative dilutions, and the relative contribution factors, a total sensitivity of the test—97% (57/59 correctly diagnosed). Total specificity of the test—49% (23/47 correctly diagnosed), as shows in Table 33.

TABLE 33
Sensitivity and specificity parameters of the training set

| | plasma results | | |
|---|---|---|---|
| Training set | healthy | patient | Total |
| Healthy | 23 | 24 | 47 |
| (by biopsy) | | | |
| % | 48.94 | 51.06 | 100.00 |
| Patient | 2 | 57 | 59 |
| (by biopsy) | | | |
| % | 3.39 | 96.61 | 100.00 |
| Total | 25 | 81 | 106 |
| % | 23.58 | 76.42 | 100.00 |

Same set of antigens was utilized on the validation set containing 15 cancer patients and 21 healthy controls (as verified by biopsies). Health status was assigned to each of the samples according to the cutoff when Z>−1.16 assignment was cancer, and Z<=−1.16 was assigned as healthy.

TABLE 34
Sensitivity and specificity parameters of the validation set

| | plasma results | | |
|---|---|---|---|
| Validation set | healthy | patient | Total |
| Healthy | 11 | 10 | 21 |
| (by biopsy) | | | |
| % | 52.38 | 47.62 | 100.00 |
| Patient | 1 | 14 | 15 |
| (by biopsy) | | | |
| % | 6.67 | 93.33 | 100.00 |
| Total | 12 | 24 | 106 |
| % | 33.33 | 66.67 | 100.00 |

Thus, using this specific relative contribution factors and discriminating/test function resulted with high sensitivity (93%) and moderate specificity (53%) in strict compliance with the clinical requirement over the breast surgeon, as shows in table 34.

In another application, the breast surgeon would like a highly specific test, with a moderately sensitive test, in order to detect as many of the false negative results of the screened population (which is about 10%).

Same procedure was performed on a set of 115 samples (64 cancer and 51 healthy). A subset containing 7 antigens resulted with the following relative contribution factor provided in table 35.

TABLE 35
Subset of 7 antigens used for diagnosis, their relative dilution rate
and the relative contribution factors calculated by logistic regression

| | Relative | Relative |
|---|---|---|
| | dilution | contribution |
| Antigen | rate | factor |
| LDPe002 | 1:32 | 4.543578 |
| LDPe012 | 1:32 | −3.195711 |
| LDPe016 | 1:32 | −3.638054 |
| LDPe066 | 1:32 | 3.839727 |
| LDPe070 | 1:32 | −4.631382 |
| LDPr077 | 1:64 | 6.163816 |
| LDPr095 | 1:64 | −4.411174 |
| Free coefficient | | 5.524788 |
| Z(cut off) | | 1.1 |

Using this specific relative contribution factors and discriminant function resulted with moderate sensitivity (50%) and high specificity (92%) in strict compliance with the clinical requirement over the breast surgeon as shows in table 36.

TABLE 36
Sensitivity and specificity parameters of the training set

| | plasma results | | |
|---|---|---|---|
| Training set | healthy | patient | Total |
| Healthy | 47 | 4 | 51 |
| (by biopsy) | | | |
| % | 92.16 | 7.84 | 100.00 |
| Patient | 32 | 32 | 64 |
| (by biopsy) | | | |
| % | 50.00 | 50.00 | 100.00 |
| Total | 79 | 36 | 106 |
| % | 68.70 | 31.30 | 100.00 |

The same set of antigen was measured on the validation set containing 18 cancer patients and 22 healthy controls (as verified by biopsies). Health status was assigned to each of the samples according to the cutoff when Z>1.1 assignment was cancer, and Z<1.1 was assigned as healthy.

TABLE 37
Sensitivity and specificity parameters of the validation set

| | plasma results | | |
|---|---|---|---|
| Validation set | healthy | patient | Total |
| Healthy | 20 | 2 | 22 |
| (by biopsy) | | | |
| % | 90.91 | 9.09 | 100.00 |
| Patient | 10 | 8 | 18 |
| (by biopsy) | | | |
| % | 55.56 | 44.44 | 100.00 |
| Total | 30 | 10 | 106 |
| % | 75.00 | 25.00 | 100.00 |

Using this specific relative contribution factors and discriminating function resulted with moderate sensitivity (44%) and high specificity (91%) in strict compliance with the clinical requirement over the breast surgeon as shows in Table 37.

1. A method of assigning a diagnosis to a subject being assessed for the presence of cancer and/or determining that a subject has an increased likelihood of being afflicted, the method comprising:
(i) providing a body sample from said subject;
(ii) contacting said sample with a predetermined set of antigens to form complexes with autoantibodies present in said sample, said autoantibodies being capable of specifically binding to said antigens;
wherein each of said antigens is characterized by a predetermined relative contribution factor to the presence of cancer;
(iii) measuring the levels of each of said antigen-antibody complexes in said subject;
(iv) determining the relative contribution parameters of each of said antigen-antibody complexes levels to the presence of cancer by adjusting each of said antigen-antibody complexes levels in accordance with the predetermined relative contribution factor;
(v) determining the output of a test function, (x)=f(relative contribution parameters);
whereby if said (x) is higher than a threshold pre-established for healthy subjects, said subject is assigned with a diagnosis that the subject has an increased likelihood of being afflicted with cancer.

2. The method of claim 1 wherein the set of antigens comprises at least two antigens and each of said antigens is characterized by a predetermined relative contribution factor to the presence of cancer in said subject; the predetermined relative contribution factors defining a relative contribution factor matrix.

3. The method of claim 2 wherein the relative contribution factor matrix comprises the proportional relationship of two or more antigen-antibody complexes levels characterizing the occurrence of cancer in said diagnosed subject.

4. The method of claim 1, wherein said sample body sample is a plasma or serum sample.

5. The method of claim 1, wherein said sample comprises a first aliquot of said sample, said first aliquot of said sample being diluted at a first dilution rate with a suitable buffer solution to provide measurable antigen-antibody complexes levels.

6. (canceled)

7. The method of claim 5, wherein said sample comprises a second aliquot of said sample, said second aliquot of said sample being diluted at a second dilution rate with a suitable buffer solution to provide measurable antigen-antibody complexes levels; the second dilution rate being different from said first dilution rate; the first and second dilution rates provide two measurable antigen-antibody complexes levels in different dilution rates;
wherein each of said two measurable antigen-antibody complexes levels is with a different antigens.

8. The method of claim 7, wherein the first and second dilution rates defines a relative dilution ratio of said two different antigens or a proportional relationship of said two different antigens.

9. The method of claim 8, wherein said relative dilution ratio comprises at least two relative dilution ratios.

10. The method of claim 1, wherein said cancer is breast or ovarian cancer.

11. The method of claim 1, wherein said cancer is colon, lung or prostate cancer.

12-16. (canceled)

17. A kit for the diagnosis of cancer in a human subject, said kit comprising:
(a) buffer solution for optionally diluting a body sample from a diagnosed subject;
(b) at least two antigens, wherein each of said antigens is characterized by a predetermined relative contribution factor to the presence of cancer; said predetermined relative contribution factors defining a relative contribution factor matrix being maintained in a register; and
(c) reagents and means for measuring antigens-autoantibodies complexes specific for said antigens in a body sample from a subject; and
(d) instructions for use.

18-20. (canceled)

21. The kit of claim 17, wherein said body sample is a plasma or serum sample.

22-25. (canceled)

26. A method of diagnosis of cancer in a subject, said method comprising the steps of: (a) providing a body sample from said subject; (b) contacting said sample with at least two different suitable antigens to form at least two different complexes with antibodies present in said sample, said antibodies being capable of specifically binding to said antigens, wherein each of said antigens is characterized by the feature that the ratio between the antibody levels specific for said at least two antigens in samples from subjects who have cancer differs from the ratio between the antibody levels specific for the same at least two antigens established for healthy subjects; (c) determining the actual levels of each of said antigen-antibody complexes in said subject; (d) establishing the ratio between the levels of said at least two different complexes in said subject; and (e) comparing said ratio to a predetermined ratio between antigen-antibody complexes levels formed between the same at least two antigens and samples from healthy subjects, whereby if said ratio determined in step (d) is higher or lower than a predetermined cutoff point pre-established for healthy subjects, said subject is diagnosed with cancer.

27. The method of claim 26, wherein said body sample is a plasma or serum sample.

28. The method of claim 26, wherein said cancer is breast or ovarian cancer.
